SEQUENCE LISTING (167 sequences)

SEQ ID NO: 1 - RNA, 25 nt, Artificial Sequence
  Description of Artificial Sequence Synthetic oligonucleotide
gaggagagcauucaaggccucguuu

SEQ ID NO: 2 - RNA, 25 nt, Artificial Sequence
  Description of Artificial Sequence Synthetic oligonucleotide
aaacgaggccuugaaugcucuccuc

SEQ ID NO: 3 - RNA, 25 nt, Artificial Sequence
  Description of Artificial Sequence Synthetic oligonucleotide
ggaacgacuccaucuuccacgauau

SEQ ID NO: 4 - RNA, 25 nt, Artificial Sequence
  Description of Artificial Sequence Synthetic oligonucleotide
auaucguggaagauggagucguucc

SEQ ID NO: 5 - DNA, 20 bp, Artificial Sequence
  Description of Artificial Sequence Synthetic primer
ggagaccacctgcttctacc

SEQ ID NO: 6 - DNA, 19 bp, Artificial Sequence
  Description of Artificial Sequence Synthetic primer
gcatacggatggacttccc

SEQ ID NO: 7 - DNA, 35 bp, Artificial Sequence
  Description of Artificial Sequence Synthetic oligonucleotide
ctatgagtgcacgtcgttaatcacaaacacacaca

SEQ ID NO: 8 - DNA, 35 bp, Artificial Sequence
  Description of Artificial Sequence Synthetic oligonucleotide
gtgtgtgtgtttgtgattaacgacgtgcactcata

SEQ ID NO: 9 - DNA, 39 bp, Artificial Sequence
  Description of Artificial Sequence Synthetic oligonucleotide
gtgataagaaataaatctaattaccatatcctttgaata

SEQ ID NO: 10 - DNA, 39 bp, Artificial Sequence
  Description of Artificial Sequence Synthetic oligonucleotide
ctattcaaaggatatggtaattagatttatttcttatca

SEQ ID NO: 11 - DNA, 29 bp, Artificial Sequence
  Description of Artificial Sequence Synthetic oligonucleotide
cttgcaaacacttaatccaaaaacgccaa

SEQ ID NO: 12 - DNA, 29 bp, Artificial Sequence
  Description of Artificial Sequence Synthetic oligonucleotide
gttggcgtttttggattaagtgtttgcaa

SEQ ID NO: 13 - DNA, 26 bp, Artificial Sequence
  Description of Artificial Sequence Synthetic oligonucleotide
aagagttcagaaggcaaccctggctc

SEQ ID NO: 14 - DNA, 22 bp, Artificial Sequence
  Description of Artificial Sequence Synthetic oligonucleotide
cagaccctccccgatttatttc

SEQ ID NO: 15 - DNA, 20 bp, Artificial Sequence
  Description of Artificial Sequence Synthetic oligonucleotide
tgttcaaaggcttcgctggg

SEQ ID NO: 16 - DNA, 26 bp, Artificial Sequence
  Description of Artificial Sequence Synthetic oligonucleotide
acacacacacacacacaaaacttcag

SEQ ID NO: 17 - DNA, 22 bp, Artificial Sequence
  Description of Artificial Sequence Synthetic oligonucleotide
atcctggcacagatcctccttc

SEQ ID NO: 18 - DNA, 27 bp, Artificial Sequence
  Description of Artificial Sequence Synthetic oligonucleotide
ccaatcagctcctagagtctcagaatc

SEQ ID NO: 19 - DNA, 22 bp, Artificial Sequence
  Description of Artificial Sequence Synthetic oligonucleotide
ggagagggtttgagagggagca

SEQ ID NO: 20 - DNA, 20 bp, Artificial Sequence
  Description of Artificial Sequence Synthetic oligonucleotide
tggggaaggtggggaggaga

SEQ ID NO: 21 - DNA, 22 bp, Artificial Sequence
  Description of Artificial Sequence Synthetic oligonucleotide
tgactcaaggtgccatagggtg

SEQ ID NO: 22 - DNA, 25 bp, Artificial Sequence
  Description of Artificial Sequence Synthetic oligonucleotide
gtgtgtgtgtgtgtgtgtgtgtttg

SEQ ID NO: 23 - DNA, 22 bp, Artificial Sequence
  Description of Artificial Sequence Synthetic oligonucleotide
ttgttcgggtctgaaggacagg

SEQ ID NO: 24 - DNA, 23 bp, Artificial Sequence
  Description of Artificial Sequence Synthetic oligonucleotide
gcaagatttccagcaagcgtatc

SEQ ID NO: 25 - DNA, 24 bp, Artificial Sequence
  Description of Artificial Sequence Synthetic oligonucleotide
tgatttatttgttcctcggagtcc

SEQ ID NO: 26 - DNA, 25 bp, Artificial Sequence
  Description of Artificial Sequence Synthetic oligonucleotide
gcgtgtgctctctgattaagtcatc

SEQ ID NO: 27 - DNA, 22 bp, Artificial Sequence
  Description of Artificial Sequence Synthetic oligonucleotide
aagaggtaaacatcttggggcg

SEQ ID NO: 28 - DNA, 25 bp, Artificial Sequence
  Description of Artificial Sequence Synthetic oligonucleotide
ggttgtcattctgggaaaatctacc

SEQ ID NO: 29 - DNA, 24 bp, Artificial Sequence
  Description of Artificial Sequence Synthetic oligonucleotide
aaatgccccctaacctcaagggag

SEQ ID NO: 30 - DNA, 27 bp, Artificial Sequence
  Description of Artificial Sequence Synthetic oligonucleotide
tggaatactagaaaagggacaaagagg

SEQ ID NO: 31 - DNA, 24 bp, Artificial Sequence
  Description of Artificial Sequence Synthetic oligonucleotide
ctctcactttttccctttcgttcg

SEQ ID NO: 32 - DNA, 20 bp, Artificial Sequence
  Description of Artificial Sequence Synthetic oligonucleotide
agtttcccccagatgttgcg

SEQ ID NO: 33 - DNA, 21 bp, Artificial Sequence
  Description of Artificial Sequence Synthetic oligonucleotide
tgtgcttgctccttggtgttc

SEQ ID NO: 34 - DNA, 24 bp, Artificial Sequence
  Description of Artificial Sequence Synthetic oligonucleotide
ctatcttcagacagtcggaaaccc

SEQ ID NO: 35 - DNA, 22 bp, Artificial Sequence
  Description of Artificial Sequence Synthetic oligonucleotide
ttgattagctccctccccagtc

SEQ ID NO: 36 - DNA, 30 bp, Artificial Sequence
  Description of Artificial Sequence Synthetic oligonucleotide
cactacaaaagtaacttatagcaaggaccc

SEQ ID NO: 37 - DNA, 24 bp, Artificial Sequence
  Description of Artificial Sequence Synthetic oligonucleotide
aaggggggattggaagttcaggtc

SEQ ID NO: 38 - DNA, 20 bp, Artificial Sequence
  Description of Artificial Sequence Synthetic oligonucleotide
gggtagcgttggcgtttttg

SEQ ID NO: 39 - DNA, 30 bp, Artificial Sequence
  Description of Artificial Sequence Synthetic oligonucleotide
ctcagataaaacagaacctaagtggaactc

SEQ ID NO: 40 - DNA, 24 bp, Artificial Sequence
  Description of Artificial Sequence Synthetic oligonucleotide
actgccttcaggagaaagtcaaag

SEQ ID NO: 41 - DNA, 23 bp, Artificial Sequence
  Description of Artificial Sequence Synthetic oligonucleotide
ccttttctagtgcattttgtggc

SEQ ID NO: 42 - DNA, 24 bp, Artificial Sequence
  Description of Artificial Sequence Synthetic oligonucleotide
ggtaatcggtttccatagcacatc

SEQ ID NO: 43 - DNA, 22 bp, Artificial Sequence
  Description of Artificial Sequence Synthetic oligonucleotide
ctgcttattttgctgtggtttg

SEQ ID NO: 44 - DNA, 22 bp, Artificial Sequence
  Description of Artificial Sequence Synthetic oligonucleotide
ccttctcttgctctcatttctg

SEQ ID NO: 45 - DNA, 22 bp, Artificial Sequence
  Description of Artificial Sequence Synthetic oligonucleotide
ccaaacaaacgcacatcatcag

SEQ ID NO: 46 - DNA, 23 bp, Artificial Sequence
  Description of Artificial Sequence Synthetic oligonucleotide
cacaaaagccagggagttcattg

SEQ ID NO: 47 - DNA, 23 bp, Artificial Sequence
  Description of Artificial Sequence Synthetic oligonucleotide
ggcagaggaacagaaagaggaag

SEQ ID NO: 48 - DNA, 22 bp, Artificial Sequence
  Description of Artificial Sequence Synthetic oligonucleotide
atgaaagtgctcgtggtgtggc

SEQ ID NO: 49 - DNA, 22 bp, Artificial Sequence
  Description of Artificial Sequence Synthetic oligonucleotide
tagtgtatccgcacaaaggggg

SEQ ID NO: 50 - DNA, 23 bp, Artificial Sequence
  Description of Artificial Sequence Synthetic oligonucleotide
aatcgctgaaccaggagacaaac

SEQ ID NO: 51 - DNA, 20 bp, Mus sp.
tactgaatcaaagtggaagg

SEQ ID NO: 52 - DNA, 20 bp, Rattus sp.
tactgaatcaaagtggaagg

SEQ ID NO: 53 - DNA, 20 bp, Homo sapiens
tactgaatcaaagtggaagg

SEQ ID NO: 54 - DNA, 20 bp, Pongo sp.
tactgaatcaaagtggaagg

SEQ ID NO: 55 - DNA, 20 bp, Canis sp.
tcctgaatcaaagtggaagg

SEQ ID NO: 56 - DNA, 20 bp, Equus ferus
tcctgaatcaaagcagaagg

SEQ ID NO: 57 - DNA, 16 bp, Mus sp.
ctgaacaaaggtgtct

SEQ ID NO: 58 - DNA, 16 bp, Rattus sp.
ctgaacaaaggtgtct

SEQ ID NO: 59 - DNA, 16 bp, Homo sapiens
ttgaacaaaggtgtct

SEQ ID NO: 60 - DNA, 16 bp, Pongo sp.
ttgaacaaaggtgtct

SEQ ID NO: 61 - DNA, 15 bp, Canis sp.
ccgaacaaaggtgcc

SEQ ID NO: 62 - DNA, 35 bp, Mus sp.
agtggaacaaagcccagcgaagcctttgaacactg

SEQ ID NO: 63 - DNA, 35 bp, Rattus sp.
agtggaacaaagcccagagaagcctttgaacactg

SEQ ID NO: 64 - DNA, 35 bp, Homo sapiens
agtggaacaaagcccagcaaagcctttgaacactg

SEQ ID NO: 65 - DNA, 35 bp, Pongo sp.; identical to SEQ ID NO: 64

SEQ ID NO: 66 - DNA, 35 bp, Canis sp.; identical to SEQ ID NO: 64

SEQ ID NO: 67 - DNA, 35 bp, Equus ferus; identical to SEQ ID NO: 64

SEQ ID NO: 68 - DNA, 14 bp, Mus sp.
cgtcgttaatcaca

SEQ ID NO: 69 - DNA, 14 bp, Rattus sp.
catggttaatcaca

SEQ ID NO: 70 - DNA, 14 bp, Homo sapiens
catatttaatcttc

SEQ ID NO: 71 - DNA, 14 bp, Pongo sp.
catatttaatcttc

SEQ ID NO: 72 - DNA, 14 bp, Canis sp.
catatttaatctcc

SEQ ID NO: 73 - DNA, 14 bp, Equus ferus
catatttaatctcc

SEQ ID NO: 74 - DNA, 13 bp, Mus sp.
agtattaaaacca

SEQ ID NO: 75 - DNA, 13 bp, Rattus sp.
attattaaaacca

SEQ ID NO: 76 - DNA, 13 bp, Homo sapiens
attattaatatta

SEQ ID NO: 77 - DNA, 13 bp, Pongo sp.
attattaatatta

SEQ ID NO: 78 - DNA, 13 bp, Canis sp.
attattagtctgt

SEQ ID NO: 79 - DNA, 15 bp, Mus sp.
tttttaatgagagaa

SEQ ID NO: 80 - DNA, 15 bp, Homo sapiens
tttttaatgagcaga

SEQ ID NO: 81 - DNA, 15 bp, Pongo sp.
tttttaatgagcaga

SEQ ID NO: 82 - DNA, 15 bp, Canis sp.
tttttaatgagtgga

SEQ ID NO: 83 - DNA, 15 bp, Equus ferus
tttttaatgagtgga

SEQ ID NO: 84 - DNA, 15 bp, Mus sp.
aaacattatttgcca

SEQ ID NO: 85 - DNA, 15 bp, Rattus sp.
aaacattatttgcca

SEQ ID NO: 86 - DNA, 15 bp, Homo sapiens
aaatattatttgctg

SEQ ID NO: 87 - DNA, 15 bp, Pongo sp.
aaatattatttgctg

SEQ ID NO: 88 - DNA, 15 bp, Canis sp.
aaatattatttgcca

SEQ ID NO: 89 - DNA, 15 bp, Equus ferus
aaatattatttgccg

SEQ ID NO: 90 - DNA, 19 bp, Mus sp.
ctttattagtttatttcag

SEQ ID NO: 91 - DNA, 19 bp, Rattus sp.
ctttattagtttatttcag

SEQ ID NO: 92 - DNA, 19 bp, Homo sapiens
ctttattagtttatttcag

SEQ ID NO: 93 - DNA, 19 bp, Pongo sp.
ctttattagtttatttcag

SEQ ID NO: 94 - DNA, 19 bp, Canis sp.
ctttattagtttatttcag

SEQ ID NO: 95 - DNA, 19 bp, Equus ferus
ctttattagtttatttcag

SEQ ID NO: 96 - DNA, 15 bp, Mus sp.
gatcattaaaacacc

SEQ ID NO: 97 - DNA, 15 bp, Rattus sp.
gatcattaaaacacc

SEQ ID NO: 98 - DNA, 15 bp, Homo sapiens
gatcattaaaacacc

SEQ ID NO: 99 - DNA, 15 bp, Pongo sp.
gatcattaaaacacc

SEQ ID NO: 100 - DNA, 15 bp, Canis sp.
gatcattaaaacacc

SEQ ID NO: 101 - DNA, 15 bp, Equus ferus
gatcattaaaacacc

SEQ ID NO: 102 - DNA, 21 bp, Mus sp.
tgggattactttgactgtggc

SEQ ID NO: 103 - DNA, 21 bp, Rattus sp.
tgggattactttgactgtggc

SEQ ID NO: 104 - DNA, 21 bp, Homo sapiens
tggtattactttgactgtggc

SEQ ID NO: 105 - DNA, 21 bp, Pongo sp.
tggtattactttgactgtggc

SEQ ID NO: 106 - DNA, 21 bp, Equus ferus
cggtattactttgactgtggc

SEQ ID NO: 107 - DNA, 16 bp, Mus sp.
gaacaacttattagtg

SEQ ID NO: 108 - DNA, 16 bp, Rattus sp.
gaacaacttattagtg

SEQ ID NO: 109 - DNA, 16 bp, Homo sapiens
gaatagcatattagca

SEQ ID NO: 110 - DNA, 16 bp, Pongo sp.
gaatagcatattagca

SEQ ID NO: 111 - DNA, 16 bp, Canis sp.
gaatagcctattagca

SEQ ID NO: 112 - DNA, 16 bp, Equus ferus
gaatagcttattagca

SEQ ID NO: 113 - DNA, 19 bp, Mus sp.
agttattattttttaatac

SEQ ID NO: 114 - DNA, 19 bp, Rattus sp.
agttattattttttaatac

SEQ ID NO: 115 - DNA, 19 bp, Homo sapiens
agttattattttttaatat

SEQ ID NO: 116 - DNA, 19 bp, Pongo sp.
agttattattttttaatat

SEQ ID NO: 117 - DNA, 19 bp, Canis sp.
gcttattattttttaatat

SEQ ID NO: 118 - DNA, 19 bp, Equus ferus
agttattattttttaatac

SEQ ID NO: 119 - DNA, 16 bp, Mus sp.
gtcattaaaaacacat

SEQ ID NO: 120 - DNA, 16 bp, Rattus sp.
gtcattaaaaacacat

SEQ ID NO: 121 - DNA, 16 bp, Homo sapiens
gccattaaaaacacat

SEQ ID NO: 122 - DNA, 16 bp, Pongo sp.
gccattaaaaacacat

SEQ ID NO: 123 - DNA, 16 bp, Equus ferus
gccattaaaaaaacac

SEQ ID NO: 124 - DNA, 21 bp, Mus sp.
taaatctaattaccatatcct

SEQ ID NO: 125 - DNA, 21 bp, Rattus sp.
taaatctaattaccatatcct

SEQ ID NO: 126 - DNA, 21 bp, Homo sapiens
taaatctaattaccatatcct

SEQ ID NO: 127 - DNA, 21 bp, Pongo sp.
taaatctaattaccatatcct

SEQ ID NO: 128 - DNA, 21 bp, Canis sp.
taaatctaattaccatatcct

SEQ ID NO: 129 - DNA, 21 bp, Equus ferus
taaatctaattaccatatcct

SEQ ID NO: 130 - DNA, 21 bp, Mus sp.
gtttttggattaagtgtttgc

SEQ ID NO: 131 - DNA, 21 bp, Rattus sp.
gtttttggattaggtatttgc

SEQ ID NO: 132 - DNA, 17 bp, Mus sp.
agtttcctaatgtggca

SEQ ID NO: 133 - DNA, 17 bp, Rattus sp.
agtttcctaatgtgcca

SEQ ID NO: 134 - DNA, 18 bp, Mus sp.
ttcaactattatcctacc

SEQ ID NO: 135 - DNA, 18 bp, Rattus sp.
ttcaactattatcccacc

SEQ ID NO: 136 - DNA, 18 bp, Mus sp.
tgatcctaatttctgagt

SEQ ID NO: 137 - DNA, 18 bp, Rattus sp.
tgatactaatttctgagt

SEQ ID NO: 138 - DNA, 17 bp, Mus sp.
tattttattaaggaaaa

SEQ ID NO: 139 - DNA, 17 bp, Rattus sp.
cattttataaaggaaaa

SEQ ID NO: 140 - DNA, 17 bp, Homo sapiens
tgttttataaaggaaaa

SEQ ID NO: 141 - DNA, 17 bp, Pongo sp.
tgttttataaaggaaaa

SEQ ID NO: 142 - DNA, 17 bp, Canis sp.
tgttttataaaggaaaa

SEQ ID NO: 143 - DNA, 17 bp, Equus ferus
tgttttataaaggaaaa

SEQ ID NO: 144 - DNA, 17 bp, Mus sp.
gatgaattattgtatgg

SEQ ID NO: 145 - DNA, 17 bp, Rattus sp.
gatgaataattgtatgg

SEQ ID NO: 146 - DNA, 17 bp, Homo sapiens
ggtaaataattggatgg

SEQ ID NO: 147 - DNA, 17 bp, Pongo sp.
actaaataattggatgg

SEQ ID NO: 148 - DNA, 17 bp, Canis sp.
ggtaaataattggatga

SEQ ID NO: 149 - DNA, 21 bp, Mus sp.
gggaattaattaccattggag

SEQ ID NO: 150 - DNA, 21 bp, Rattus sp.
gggaattaattaccattggag

SEQ ID NO: 151 - DNA, 21 bp, Homo sapiens
gggaattaattaccattggag

SEQ ID NO: 152 - DNA, 21 bp, Pongo sp.
gggaattaattaccattggag

SEQ ID NO: 153 - DNA, 21 bp, Canis sp.
gggaattaattaccattggag

SEQ ID NO: 154 - DNA, 21 bp, Equus ferus
gggaattaattaccattggag

SEQ ID NO: 155 - DNA, 21 bp, Mus sp.
ttcctgtaattatttggtgtc

SEQ ID NO: 156 - DNA, 21 bp, Rattus sp.
ttcctgtaattatttggtgtc

SEQ ID NO: 157 - DNA, 21 bp, Homo sapiens
ttcctgtaattatttggtgtc

SEQ ID NO: 158 - DNA, 21 bp, Pongo sp.
ttcctataattatttggtgtc

SEQ ID NO: 159 - DNA, 21 bp, Canis sp.
ttcctgtaattatttggtgtc

SEQ ID NO: 160 - DNA, 41 bp, Mus sp.
cctctttaatagaatggatgtgctatggaaaccgattaccc

SEQ ID NO: 161 - DNA, 41 bp, Rattus sp.
cctctttaatagaatggatgtgctatgaaaaccgattaccc

SEQ ID NO: 162 - DNA, 41 bp, Homo sapiens
cctctttaatagaatgaatgtgctatgaaaaccgattaccc

SEQ ID NO: 163 - DNA, 41 bp, Pongo sp.; identical to SEQ ID NO: 162

SEQ ID NO: 164 - DNA, 41 bp, Canis sp.; identical to SEQ ID NO: 162

SEQ ID NO: 165 - DNA, 41 bp, Equus ferus; identical to SEQ ID NO: 162

SEQ ID NO: 166 - protein, 9 aa, Artificial Sequence
  Description of Artificial Sequence Synthetic peptide
RRRRRRRRR

SEQ ID NO: 167 - protein, 6 aa, Artificial Sequence
  Description of Artificial Sequence Synthetic 6xHis tag
HHHHHH

(19) United States
(12) Patent Application Publication
(10) Pub. No.: US 2013/0052268 A1
(43) Pub. Date: Feb. 28, 2013

(54) COMPOSITIONS AND METHODS FOR TREATMENT OF PARKINSON'S DISEASE

(71) Applicants: Sangmi Chung, Belmont, MA (US); Kwang-Soo Kim, Lexington, MA (US)

(72) Inventors: Sangmi Chung, Belmont, MA (US); Kwang-Soo Kim, Lexington, MA (US)

(73) Assignee: THE MCLEAN HOSPITAL CORPORATION, Belmont, MA (US)

(21) Appl. No.: 13/578,831

(22) PCT Filed: Jun. 1, 2010
(86) PCT No.: PCT/US10/36954

§ 371 Date: Oct. 31, 2012

Related U.S. Application Data
(60) Provisional application No. 61/305,099, filed on Feb. 16, 2010.

(51) Int. Cl. A61K 31/711; C12N 5/0793; A61P 25/16; A61K 38/16; A61K 9/48

(52) U.S. Cl. 424/490; 514/44 R; 514/17.7; 435/377

(57) ABSTRACT

The present invention relates to methods for producing neural cells from progenitor or stem cells by activating both the Wnt1-Lmx1a/Lmx1b and the SHH-FoxA2 signaling pathways by, for example, increasing the biological activity of one or more of Wnt1, Lmx1a, Lmx1b, Otx2 and Pitx3 and one or more of SHH, FoxA2 and Nurr1 in the progenitor or stem cells including embryonic stem cells and iPS cells. Such cells may be used for the treatment of Parkinson's disease. The invention further relates to methods for treating Parkinson's disease by increasing the biological activity of one or more of Wnt1, Lmx1b, Lmx1b, Otx2 and Pitx3 and one or more of SHH, FoxA2 and Nurr1 in the midbrain of a patient. In particular, the biological activity of the proteins can be increased by virtue of a cell penetrating peptide fused to the proteins or by transfecting RNAs encoding the proteins such that the host chromosomal DNAs remain intact.

### STATEMENT OF GOVERNMENT SUPPORT

This invention was made with United States government support awarded by the following agency: NIH (P50 NS39793 and MH48866). The United States government has certain rights in the invention.

### FIELD OF THE INVENTION

The present technology relates generally to the compositions and methods for treatment of neurodegenerative diseases, including Parkinson's Disease.

### BACKGROUND OF THE INVENTION

The following description is provided to assist the understanding of the reader. None of the information provided or references cited is admitted to be prior art to the present invention. Throughout this disclosure, various publications, patents and published patent specifications are referenced by an identifying citation. The disclosures of these publications, patents and published patent specifications are hereby incorporated by reference in their entirety into the present disclosure, thereby to more fully describe the state of the art to which this invention pertains.

Parkinson's disease (PD) is a progressive neurodegenerative disease characterized clinically by bradykinesia, rigidity, and resting tremor. Selective degeneration of specific neuronal populations is a universal feature of PD that contributes to the clinical symptomology which is poorly understood. The hallmark neuropathologic feature of PD is loss of midbrain dopaminergic (mDA) neurons.

Midbrain dopaminergic (mDA) neurons critically control voluntary movement, emotion, and reward through specific neuronal circuits (Bjorklund and Lindvall, 1984), and their selective degeneration and/or dysregulation is associated with major neurological and psychiatric disorders. Selective loss of mDA neurons in the substantia nigra is associated with PD (Lang and Lozano, 1998). Successful cell replacement therapy for PD requires generation of optimal cell sources. There has been extensive effort to generate mDA neurons from stem cells (Chung et al., 2002; Kawasaki et al., 2000; Kim et al., 2002).

During early brain development, mDA neurons originate from the ventral midline of the mesencephalon. The initial event of mDA neuron development was shown to depend on Sonic hedgehog (SHH), fibroblast growth factor 8 (FGF8), and Wnt1, setting up the initial field for mDA progenitors (McMahon and Bradley, 1990; Prakash et al., 2006; Ye et al., 1998). Among these, Wnt1 and FGF8 are expressed from Isthmus and they cross regulate each other (Chi et al., 2003; Lee et al., 1997; Liu and Joyner, 2001; Matsunaga et al., 2002). Recent studies showing that FGF8 failed to induce ectopic DA neurons in Wnt1 mutant embryos (Prakash et al., 2006) suggest that Wnt1, which can be induced by FGF8, is a more direct regulator of initiation of mDA fields. Furthermore, a recent study established that compound FGFR mutant mice show that FGF8 regulates mDA neuronal precursors (NP) proliferation rather than mDA identity, the latter being more critically mediated by SHH and Wnt1 (Saarimaki-Vire et al., 2007). SHH expressed from the notochord has been shown to directly induce FoxA2 expression in ventral mesencephalon (VM) through Gli binding sites in the FoxA2 gene (Sasaki et al., 1997). FoxA2, in turn, directly induces VM SHH expression through well-conserved FoxA2 binding sites in the SHH gene (Jeong and Epstein, 2003). FoxA2 regulates mDA development by inhibiting an alternate fate (Nkx2.2+ cells), inducing neurogenesis through Ngn2, and regulating Nurr1 and DA phenotype genes (Ferri et al., 2007) as well as regulating survival/maintenance of mDA neurons (Arenas, 2008; Kittappa et al., 2007), strongly suggesting that FoxA2 is the main mediator of SHH signaling in mDA development. These extrinsic signals are thought to initiate the regulatory cascades leading to mDA development by inducing key transcription factors.

### SUMMARY OF THE INVENTION

The present inventions are based on the discovery that there exists a tight autoregulatory loop between Wnt1 and Lmx1a during mDA differentiation of embryonic stem (ES) cells as well as during embryonic midbrain development. This autoregulatory loop, in turn, directly regulates Otx2 expression, through the canonical Wnt signaling pathway, and Nurr1 and Pitx3 expression, through Lmx1a. It has also been discovered that activation of both the Wnt1-Lmx1a and the SHH-FoxA2 signaling pathways, by exogenous expression of direct downstream targets of these pathways (e.g., Otx2, Lmx1a and FoxA2), can synergistically induce mDA differentiation and inhibit differentiation into other neural cell types and therefore effectively produce a mDA population of high purity.

Accordingly, in one aspect, the invention provides a method for treating or preventing Parkinson's Disease in a patient, by increasing the level (e.g., the intracellular amount) of at least one protein of the Wnt1-Lmx1a signaling pathway selected from the group consisting of Wnt1, Lmx1a, Lmx1b, Otx2 and Pitx3 and at least one protein of the SHH-FoxA2 signaling pathway selected from the group consisting of SHH, FoxA2 and Nurr1 in the midbrain dopaminergic neurons of the patient. Preferably, the biological activity of the proteins is increased in the dopaminergic neurons of the substantia nigra of the patient (e.g., the A9 region). In one embodiment, the patient is administered one or more vectors which encodes and is capable of expressing the proteins (e.g., at least operably linked to a promoter). Suitable vectors include viral vectors such as adenoviral, adeno-associated viral, lentiviral, and retroviral vectors. Suitable promoters include neuron-specific promoters (e.g., the neural specific enolase promoter) or promoters normally found in dopaminergic neurons (e.g., promoters from genes encoding tyrosine hydroxylase, DAT, and DDC). In other embodiments, the proteins are administered to the patient. Optionally, the proteins may be encapsulated (e.g., in liposomes) to facilitate uptake by the target cells.

Various combinations of proteins from the Wnt1-Lmx1a and the SHH-FoxA2 signaling pathways are suitable for practicing the inventions. In one embodiment, the method comprises increasing the biological activity of FoxA2, Lmx1a and/or Otx2. In another embodiment, the method comprises increasing the biological activity of Nurr1, Pitx3 and/or Lmx1a. In yet another embodiment, the method comprises increasing the biological activity of Nurr1, Pitx3, Lmx1a, FoxA2 and/or Otx2.

In some embodiments, the method further comprises increasing the biological activities of one or more proteins selected from En1, En2 and Ngn2.

In another aspect, the invention provides a method for producing a neural cell from neural progenitor cells or stem cells by increasing the biological activity of at least one protein of the Wnt1-Lmx1a signaling pathway selected from the group consisting of Wnt1, Lmx1a, Lmx1b, Otx2 and Pitx3 and at least one protein of the SHH-FoxA2 signaling pathway selected from the group consisting of SHH, FoxA2 and Nurr1 in the progenitor cells under conditions suitable to produce a neural cell (e.g., a dopaminergic neuron). Preferably, the neural cells express one or more of TH, DAT, and DDC. The biological activity of proteins may be increased by contacting the progenitor cells with any of the vectors described above, under conditions suitable for the cells to take up the vector and express the proteins.

In some embodiments, the biological activity of the proteins is increased by directly delivering the proteins to the neural progenitor cells or stem cells, or delivering to the neural progenitor cells (e.g., stem cells) mRNA encoding these proteins. In some embodiments, the proteins each is attached to a cell penetrating peptide (CPP).

Non-limiting examples of combinations of proteins from the Wnt1-Lmx1a and the SHH-FoxA2 signaling pathways suitable for practicing the invention are described above.

In one aspect, the cells produced by the foregoing methods may be used to treat or prevent Parkinson's disease in a patient. The cells are administered to the patient in a therapeutically-effective manner including, for example, by transplantation into the midbrain (e.g., the substantia nigra, preferably in or adjacent to the A9 region) of the patient. Optionally, the cells are encapsulated prior to implantation.

In another aspect, the invention provides modified polypeptides useful for producing neural cells from progenitor cells. The polypeptides include the various Wnt1-Lmx1a signaling pathway members (e.g., Wnt1, Lmx1a, Lmx1b, Otx2 and Pitx3) and the various SHH-FoxA2 signaling pathway members (e.g., SHH, FoxA2 and Nurr1), fused to a cell penetrating peptide (CPP). In some embodiments, the CPP is fused to the C-terminus of the proteins either directly or through a linker (e.g., an amino acid or polymer linker). Suitable CPPs include, for example, the HIV TAT protein or any polycationic polypeptide or polymer (e.g., at least five consecutive arginine residues). One, two, three, four, five, or more of these polypeptides may be incorporated into a pharmaceutical formulation which itself may be administered to a patient, in a therapeutically effective amount, for the treatment or prevention of Parkinson's Disease.

As used herein, “stem cell” defines a cell with the ability to divide for indefinite periods in culture and give rise to specialized cells. Stem cells include, for example, somatic (adult) and embryonic stem cells. A somatic stem cell is an undifferentiated cell found in a differentiated tissue that can renew itself (clonal) and (with certain limitations) differentiate to yield all the specialized cell types of the tissue from which it originated. An embryonic stem cell is a primitive (undifferentiated) cell derived from the embryo that has the potential to become a wide variety of specialized cell types. An embryonic stem cell is one that has been cultured under in vitro conditions that allow proliferation without differentiation. Non-limiting examples of embryonic stem cells are the HES2 (also known as ES02) cell line available from ESI, Singapore and the H1 (also know as WA01) cell line available from WiCells, Madison, Wis. In addition, for example, there are 40 embryonic stem cell lines that are recently approved for use in NIH-funded research including CHB-1, CHB-2, CHB-3, CHB-4, CHB-5, CHB-6, CHB-8, CHB-9, CHB-10, CHB-11, CHB-12, RUES1, HUES1, HUES2, HUES3, HUES4, HUES5, HUES6, HUES7, HUES8, HUES9, HUES10, HUES11, HUES12, HUES13, HUES14, HUES15, HUES16, HUES17, HUES18, HUES19, HUES20, HUES21, HUES22, HUES23, HUES24, HUES26, HUES27, and HUES28. Pluripotent embryonic stem cells can be distinguished from other types of cells by the use of markers including, but not limited to, Oct-4, alkaline phosphatase, CD30, TDGF-1, GCTM-2, Genesis, Germ cell nuclear factor, SSEA1, SSEA3, and SSEA4.

As used herein, a “pluripotent cell” broadly refers to stem cells with similar properties to embryonic stem cells with respect to the ability for self-renewal and pluripotentcy (i.e., the ability to differentiate into cells of multiple lineages). Pluripotent cells refer to cells both of embryonic and non-embryonic origin. For example, pluripotent cells includes Induced Pluripotent Stem Cells (iPSCs).

An “induced pluripotent stem cell” or “iPSC” or “iPS cell” refers to an artificially derived stem cell from a non-pluripotent cell, typically an adult somatic cell, produced by inducing expression of one or more reprogramming genes or corresponding proteins or RNAs. Such stem cell specific genes include, but are not limited to, the family of octamer transcription factors, i.e. Oct-3/4; the family of Sox genes, i.e. Sox1, Sox2, Sox3, Sox 15 and Sox 18; the family of Klf genes, i.e. Klf1, Klf2, Klf4 and Klf5; the family of Myc genes, i.e. c-myc and L-myc; the family of Nanog genes, i.e. OCT4, NANOG and REX1; or LIN28. Examples of iPSCs and methods of preparing them are described in Takahashi et al. Cell 131(5):861-72, 2007; Takahashi & Yamanaka Cell 126:663-76, 2006; Okita et al. Nature 448:260-262, 2007; Yu et al. Science 318(5858):1917-20, 2007; and Nakagawa et al. Nat. Biotechnol. 26(1):101-6, 2008.

A “multi-lineage stem cell” or “multipotent stem cell” refers to a stem cell that reproduces itself and at least two further differentiated progeny cells from distinct developmental lineages. The lineages can be from the same germ layer (i.e. mesoderm, ectoderm or endoderm), or from different germ layers. An example of two progeny cells with distinct developmental lineages from differentiation of a multilineage stem cell is a myogenic cell and an adipogenic cell (both are of mesodermal origin, yet give rise to different tissues). Another example is a neurogenic cell (of ectodermal origin) and adipogenic cell (of mesodermal origin).

A neural stem cell is a cell that can be isolated from the adult central nervous systems of mammals, including humans. They have been shown to generate neurons, migrate and send out aconal and dendritic projections and integrate into pre-existing neuroal circuits and contribute to normal brain function. Reviews of research in this area are found in Miller Brain Res. 1091(1):258-264, 2006; Pluchino et al. Brain Res. Brain Res. Rev. 48(2):211-219, 2005; and Goh, et al. Stem Cell Res., 12(6):671-679, 2003. Neural stem cells can be identified and isolated by neural stem cell specific markers including, but limited to, CD133, ICAM-1, MCAM, CXCR4 and Notch 1. Neural stem cells can be isolated from animal or human by neural stem cell specific markers with methods known in the art. See, e.g., Yoshida et al., (2006). Stem Cells 24(12):2714-22.

A “precursor” or “progenitor cell” intends to mean cells that have a capacity to differentiate into a specific type of cell. A progenitor cell may be a stem cell. A progenitor cell may also be more specific than a stem cell. A progenitor cell may be unipotent or multipotent. Compared to adult stem cells, a progenitor cell may be in a later stage of cell differentiation. Examples of progenitor cells include, but are not limited to, satellite cells found in muscles, intermediate progenitor cells formed in the subventricular zone, bone marrow stromal cells, periosteum progenitor cells, pancreatic progenitor cells and angioblasts or endothelial progenitor cells. Examples of progenitor cells may also include, but are not limited to, epidermal and dermal cells from neonatal organisms.

A “neural precursor cell”, “neural progenitor cell” or “NP cell” refers to a cell that has a capacity to differentiate into a neural cell or neuron. A NP cell can be an isolated NP cell, or derived from a stem cell including but not limited to an iPS cell. Neural precursor cells can be identified and isolated by neural precursor cell specific markers including, but limited to, nestin and CD133. Neural precursor cells can be isolated from animal or human tissues such as adipose tissue (see, e.g., Vindigni et al., (2009) Neurol. Res. 2009 Aug. 5. [Epub ahead of print]) and adult skin (see, e.g., Joannides (2004) Lancet. 364(9429):172-8). Neural precursor cells can also be derived from stem cells or cell lines or neural stem cells or cell lines. See generally, e.g., U.S. Patent Application Publications Nos: 2009/0263901, 2009/0263360 and 2009/0258421.

A population of cells intends a collection of more than one cell that is identical (clonal) or non-identical in phenotype and/or genotype.

As used herein, the term “oligonucleotide” or “polynucleotide” refers to a short polymer composed of deoxyribonucleotides, ribonucleotides or any combination thereof. Oligonucleotides are generally at least about 10, 15, 20, 25, 30, 40, 50, 60, 70, 80, 90, 100 or more nucleotides in length. An oligonucleotide may be used as a primer or as a probe.

As used herein, the term “promoter” refers to a nucleic acid sequence sufficient to direct transcription of a gene. Also included in the invention are those promoter elements which are sufficient to render promoter dependent gene expression controllable for cell type specific, tissue specific or inducible by external signals or agents. The term “neuron-specific promoter” refers to a promoter that results in a higher level of transcription of a gene in cells of neuronal lineage compared to the transcription level observed in cells of a non-neuronal lineage. Examples of neuron-specific promoters useful in the methods and compositions described herein include the promoter from neuron-specific enolase (NSE) and the dopamine transporter (DAT).

As used herein, the term “regulatory element” refers to a nucleic acid sequence capable of modulating the transcription of a gene. Non-limiting examples of regulatory element include promoter, enhancer, silencer, poly-adenylation signal, transcription termination sequence. Regulatory element may be present 5′ or 3′ regions of the native gene, or within an intron.

Various proteins are also disclosed herein with their GenBank Accession Numbers for their human proteins and coding sequences. However, the proteins are not limited to human-derived proteins having the amino acid sequences represented by the disclosed GenBank Accession Nos, but may have an amino acid sequence derived from other animals, particularly, a warm-blooded animal (e.g., rat, guinea pig, mouse, chicken, rabbit, pig, sheep, cow, monkey, etc.).

As used herein, the term “Otx2” or “Orthodenticle homolog 2” refers to a protein having an amino acid sequence substantially identical to the Otx2 sequence of GenBank Accession No. AAD31385. A suitable cDNA encoding Otx2 is provided at GenBank Accession No. AF093138.

As used herein, the term “biological activity of Otx2” refers to any biological activity associated with the full length native Otx2 protein. In one embodiment, the biological activity of Otx2 refers to transcriptional activation of genes that relate to axon guidance cues, including, but not limited to neuropilin 1, neuropilin 2, slit 2, and adenylyl cyclase activating peptide. In one embodiment, the Otx2 biological activity refers to the action of protecting dopaminergic neurons from various insults, including MPP⁺ toxicity. In suitable embodiments, the Otx2 biological activity is equivalent to the activity of a protein having an amino acid sequence represented by GenBank Accession No. AAD31385. Measurement of transcriptional activity can be performed using any known method, such as a reporter assay or RT-PCR.

As used herein, the term “Lmx1a” or “LIM homeobox transcription factor 1, alpha” refers to a protein having an amino acid sequence substantially identical to the Lmx1a sequence of GenBank Accession No. NP_{—}796372. A suitable cDNA encoding Lmx1a is provided at GenBank Accession No. NM_{—}177398.

As used herein, the term “biological activity of Lmx1a” refers to any biological activity associated with the full length native Lmx1a protein. In one embodiment, the biological activity of Lmx1a refers to transcriptional activation of genes having an A/T-rich sequence, the FLAT element. Non-limiting examples of these genes include Wnt1, Msx1, Nurr1 and Pitx3. In one embodiment, the biological activity of Lmx1a refers to the induction of differentiation of neural precursor cells to midbrain dopamine neurons. In suitable embodiments, the biological activity of Lmx1a is equivalent to the activity of a protein having an amino acid sequence represented by GenBank Accession No. NP_{—}796372. Measurement of transcriptional activity can be performed using any known method, such as a reporter assay or RT-PCR.

As used herein, the term “Lmx1b” or “LIM homeobox transcription factor 1, beta” refers to a protein having an amino acid sequence substantially identical to the Lmx1b sequence of GenBank Accession No. NP_{—}002307. A suitable cDNA encoding Lmx1b is provided at GenBank Accession No. NM_{—}002316.

As used herein, the term “biological activity of Lmx1b” refers to any biological activity associated with the full length native Lmx1b protein. In one embodiment, the biological activity of Lmx1b refers to transcriptional activation of genes. Non-limiting examples of these genes include Wnt1, Msx1, Nurr1 and Pitx3. In one embodiment, the biological activity of Lmx1b refers to the induction of differentiation of neural precursor cells to midbrain dopamine neurons. In suitable embodiments, the biological activity of Lmx1b is equivalent to the activity of a protein having an amino acid sequence represented by GenBank Accession No. NP_{—}002307. Measurement of transcriptional activity can be performed using any known method, such as a reporter assay or RT-PCR.

As used herein, the term “FoxA2” or “forkhead box A2”, including isoforms 1 and 2, refers to a protein having an amino acid sequence substantially identical to the FoxA2 sequences of GenBank Accession Nos. NP_{—}068556 (isoform 1) and NP_{—}710141 (isoform 2). Suitable cDNA encoding FoxA2 are provided at GenBank Accession Nos. NM_{—}021784 (isoform 1) and NM_{—}153675 (isoform 2).

As used herein, the term “biological activity of FoxA2” refers to any biological activity associated with the full length native FoxA2 protein. In one embodiment, the biological activity of FoxA2 refers to forkhead class of DNA-binding capability. In another embodiment, the biological activity of FoxA2 transcriptional activation of genes including but not limited to Nurr1, SHH, Ngn2 and Nkx6.1 or suppression of Nkx2.2. In one embodiment, the biological activity of FoxA2 refers to the induction of differentiation of neural precursor cells to midbrain dopamine neurons. In suitable embodiments, the biological activity of FoxA2 is equivalent to the activity of a protein having an amino acid sequence represented by GenBank Accession Nos. NP_{—}068556 or NP_{—}710141. Measurement of transcriptional activity can be performed using any known method, such as a reporter assay or RT-PCR.

As used herein, the term “FoxA1” or “forkhead box A1”, refers to a protein having an amino acid sequence substantially identical to the FoxA1 sequence of GenBank Accession No. NP_{—}004487. Suitable cDNA encoding FoxA1 is provided at GenBank Accession No. NM_{—}004496.

As used herein, the term “biological activity of FoxA1” refers to any biological activity associated with the full length native FoxA1 protein. In one embodiment, the biological activity of FoxA1 refers to forkhead class of DNA-binding capability. In another embodiment, the biological activity of FoxA1 transcriptional activation of genes including but not limited to alpha-fetoprotein (AFP), albumin, tyrosine aminotransferase, phosphoenolpyruvate carboxykinase 2 (PEPCK), Nurr1, SHH, Ngn2 and Nkx6.1 or suppression of Nkx2.2. In one embodiment, the biological activity of FoxA1 refers to the induction of differentiation of neural precursor cells to midbrain dopamine neurons. In suitable embodiments, the biological activity of FoxA1 is equivalent to the activity of a protein having an amino acid sequence represented by GenBank Accession No. NP_{—}004487. Measurement of transcriptional activity can be performed using any known method, such as a reporter assay or RT-PCR.

As used herein, the term “Wnt1” or “wingless-type MMTV integration site family, member 1”, refers to a protein having an amino acid sequence substantially identical to the Wnt1 sequence of GenBank Accession No. NP_{—}005421. Suitable cDNA encoding Wnt1 is provided at GenBank Accession No. NM_{—}005430.

As used herein, the term “biological activity of Wnt1” refers to any biological activity associated with the full length native Wnt1 protein. In one embodiment, the biological activity of Wnt1 refers to the general transcriptional activation capability of Wnt family of proteins. In another embodiment, the biological activity of Wnt1 transcriptional activation of genes including but not limited to Lmx1a, Lmx1b and Otx2 and suppression of SHH. In one embodiment, the biological activity of Wnt1 refers to the induction of differentiation of neural precursor cells to midbrain dopamine neurons. In suitable embodiments, the biological activity of Wnt1 is equivalent to the activity of a protein having an amino acid sequence represented by GenBank Accession No. NP_{—}005421. Measurement of transcriptional activity can be performed using any known method, such as a reporter assay or RT-PCR.

As used herein, the term “SHH” or “sonic hedgehog homolog”, refers to a protein having an amino acid sequence substantially identical to the SHH sequence of GenBank Accession No. NP_{—}000184. Suitable cDNA encoding SHH is provided at GenBank Accession No. NM_{—}000193.

As used herein, the term “biological activity of SHH” refers to any biological activity associated with the full length native SHH protein. In one embodiment, the biological activity of SHH refers to binding to the patched (PTC) receptor, which functions in association with smoothened (SMO), to activate the transcription of target genes. In another embodiment, the biological activity of SHH transcriptional activation of genes including but not limited to FoxA1, FoxA2 and Nkx2.2. In one embodiment, the biological activity of SHH refers to the induction of differentiation of neural precursor cells to midbrain dopamine neurons. In suitable embodiments, the biological activity of SHH is equivalent to the activity of a protein having an amino acid sequence represented by GenBank Accession No. NP_{—}000184. Measurement of transcriptional activity can be performed using any known method, such as a reporter assay or RT-PCR.

As used herein, the term “Msx1” or “msh homeobox 1”, refers to a protein having an amino acid sequence substantially identical to the Msx1 sequence of GenBank Accession No. NP_{—}002439. Suitable cDNA encoding Msx1 is provided at GenBank Accession No. NM_{—}002448.

As used herein, the term “biological activity of Msx1” refers to any biological activity associated with the full length native Msx1 protein. Msx1 may act as a transcriptional repressor or activator. In another embodiment, the biological activity of Msx1 includes repression of Nkx6.1 or activation of Ngn2. In one embodiment, the biological activity of Msx1 refers to the induction of differentiation of neural precursor cells to midbrain dopamine neurons or repression of the neural precursor cells to differentiate to other neural cell types. In suitable embodiments, the biological activity of Msx1 is equivalent to the activity of a protein having an amino acid sequence represented by GenBank Accession No. NP_{—}002439. Measurement of transcriptional activity can be performed using any known method, such as a reporter assay or RT-PCR.

As used herein, the term “Ngn2” or “neurogenin 2”, refers to a protein having an amino acid sequence substantially identical to the Ngn2 sequence of GenBank Accession No. NP_{—}076924. Suitable cDNA encoding Ngn2 is provided at GenBank Accession No. NM_{—}024019.

As used herein, the term “biological activity of Ngn2” refers to any biological activity associated with the full length native Ngn2 protein. Ngn2 is a member of the neurogenin subfamily of basic helix-loop-helix (bHLH) transcription factor genes that play an important role in neurogenesis from migratory neural crest cells. In one embodiment, the biological activity of Ngn2 refers to the promotion of proliferation of neural cells. In suitable embodiments, the biological activity of Ngn2 is equivalent to the activity of a protein having an amino acid sequence represented by GenBank Accession No. NP_{—}076924. Measurement of transcriptional activity can be performed using any known method, such as a reporter assay or RT-PCR.

As used herein, the term “Pitx3” or “paired-like homeodomain 3”, refers to a protein having an amino acid sequence substantially identical to the Pitx3 sequence of GenBank Accession No. NP_{—}005020. Suitable cDNA encoding Pitx3 is provided at GenBank Accession No. NM_{—}005029.

As used herein, the term “biological activity of Pitx3” refers to any biological activity associated with the full length native Pitx3 protein. Pitx3 plays a role in neural development and is a cell marker for mDA. In one embodiment, the biological activity of Pitx3 refers to the induction of differentiation of neural precursor cells to midbrain dopamine neurons. In suitable embodiments, the biological activity of Pitx3 is equivalent to the activity of a protein having an amino acid sequence represented by GenBank Accession No. NP_{—}005020. Measurement of transcriptional activity can be performed using any known method, such as a reporter assay or RT-PCR.

As used herein, the term “Nurr1” or “nuclear receptor subfamily 4, group A, member 2”, refers to a protein having an amino acid sequence substantially identical to the Nurr1 sequence of GenBank Accession No. NP_{—}006177. Suitable cDNA encoding Nurr1 is provided at GenBank Accession No. NM_{—}006186.

As used herein, the term “biological activity of Nurr1” refers to any biological activity associated with the full length native Nurr1 protein. Nurr1 plays a role in neural development and is a cell marker for mDA. Nurr1 is a nuclear receptor and may function as a general coactivator of gene transcription. In one embodiment, the biological activity of Nurr1 refers to the induction of differentiation of neural precursor cells to midbrain dopamine neurons. In suitable embodiments, the biological activity of Nurr1 is equivalent to the activity of a protein having an amino acid sequence represented by GenBank Accession No. NP_{—}006177. Measurement of transcriptional activity can be performed using any known method, such as a reporter assay or RT-PCR.

As used herein, the term “Nkx2.2” or “NK2 homeobox 2”, refers to a protein having an amino acid sequence substantially identical to the Nkx2.2 sequence of GenBank Accession No. NP_{—}002500. Suitable cDNA encoding Nkx2.2 is provided at GenBank Accession No. NM_{—}002509.

As used herein, the term “biological activity of Nkx2.2” refers to any biological activity associated with the full length native Nkx2.2 protein. Nkx2.2 plays a role in neural development. In one embodiment, the biological activity of Nkx2.2 refers to the induction of differentiation of neural precursor cells to neurons other than midbrain dopamine neurons. In suitable embodiments, the biological activity of Nkx2.2 is equivalent to the activity of a protein having an amino acid sequence represented by GenBank Accession No. NP_{—}002500. Measurement of transcriptional activity can be performed using any known method, such as a reporter assay or RT-PCR.

As used herein, the term “Nkx6.1” or “NK6 homeobox 1”, refers to a protein having an amino acid sequence substantially identical to the Nkx6.1 sequence of GenBank Accession No. NP_{—}006159. Suitable cDNA encoding Nkx6.1 is provided at GenBank Accession No. NM_{—}006168.

As used herein, the term “biological activity of Nkx6.1” refers to any biological activity associated with the full length native Nkx6.1 protein. Nkx6.1 plays a role in neural development. In one embodiment, the biological activity of Nkx6.1 refers to the induction of differentiation of neural precursor cells to neurons other than midbrain dopamine neurons. In suitable embodiments, the biological activity of Nkx6.1 is equivalent to the activity of a protein having an amino acid sequence represented by GenBank Accession No. NP_{—}006159. Measurement of transcriptional activity can be performed using any known method, such as a reporter assay or RT-PCR.

As used herein, the term “TH” or “tyrosine hydroxylase”, refers to a protein having an amino acid sequence substantially identical to the TH sequence of GenBank Accession No. NP_{—}954986 (isoform a), NP_{—}000351 (isoform b) or NP_{—}954987 (isoform c). Suitable cDNA encoding Nkx6.1 is provided at GenBank Accession No. NM_{—}199292 (isoform a), NM_{—}000360 (isoform b) or NM_{—}199293 (isoform c). TH is a protein marker of mDA.

As used herein, the term “DAT” or “dopamine transporter”, refers to a protein having an amino acid sequence substantially identical to the DAT sequence of GenBank Accession No. NP_{—}001035. Suitable cDNA encoding DAT is provided at GenBank Accession No. NM_{—}001044. DAT is a protein marker for mDA.

As used herein, the term “DDC” or “DOPA decarboxylase”, refers to a protein having an amino acid sequence substantially identical to the DDC sequence of GenBank Accession No. NP_{—}000781. Suitable cDNA encoding DDC is provided at GenBank Accession No. NM_{—}000790. DDC is a protein marker for mDA.

As used herein, the term “FGF8” or “fibroblast growth factor 8”, refers to a protein having an amino acid sequence substantially identical to the FGF8 sequence of GenBank Accession No. NP_{—}149355. Suitable cDNA encoding DDC is provided at GenBank Accession No. NM_{—}033165. FGF8 plays a role in inducing and promoting mDA differentiation through activation of En1/2. See, e.g., Abeliovich and Hammond, Developmental Biology, 304:447-54 (2007).

As used herein, the term “En1” or “engrailed homeobox 1”, refers to a protein having an amino acid sequence substantially identical to the En1 sequence of GenBank Accession No. NP_{—}001417. Suitable cDNA encoding En1 is provided at GenBank Accession No. NM_{—}001426. As used herein, the term “biological activity of En1” refers to any biological activity associated with the full length native En1 protein

As used herein, the term “En2” or “engrailed homeobox 2”, refers to a protein having an amino acid sequence substantially identical to the En2 sequence of GenBank Accession No. NP_{—}001418. Suitable cDNA encoding DDC is provided at GenBank Accession No. NM_{—}001427. As used herein, the term “biological activity of En2” refers to any biological activity associated with the full length native En2 protein

As used herein, the term “treating” is meant administering a pharmaceutical composition for the purpose of improving the condition of a patient by reducing, alleviating, reversing, or preventing at least one adverse effect or symptom.

As used herein, the term “preventing” is meant identifying a subject (i.e., a patient) having an increased susceptibility to PD but not yet exhibiting symptoms of the disease, and administering a therapy according to the principles of this disclosure. The preventive therapy is designed to reduce the likelihood that the susceptible subject will later become symptomatic or that the disease will be delay in onset or progress more slowly than it would in the absence of the preventive therapy. A subject may be identified as having an increased likelihood of developing PD by any appropriate method including, for example, by identifying a family history of PD or other degenerative brain disorder, or having one or more diagnostic markers indicative of disease or susceptibility to disease.

As used herein, the term “sample” or “test sample” refers to any liquid or solid material containing nucleic acids. In suitable embodiments, a test sample is obtained from a biological source (i.e., a “biological sample”), such as cells in culture or a tissue sample from an animal, most preferably, a human. Exemplary sample tissues include, but are not limited to, blood, bone marrow, body fluids, cerebrospinal fluid, plasma, serum, or tissue (e.g. biopsy material).

“Target nucleic acid” as used herein refers to segments of a chromosome, a complete gene with or without intergenic sequence, segments or portions a gene without intergenic sequence, or sequence of nucleic acids to which probes or primers are designed. Target nucleic acids may include wild type sequences, nucleic acid sequences containing mutations, deletions or duplications, tandem repeat regions, a gene of interest, a region of a gene of interest or any upstream or downstream region thereof. Target nucleic acids may represent alternative sequences or alleles of a particular gene. Target nucleic acids may be derived from genomic DNA, cDNA, or RNA. As used herein, target nucleic acid may be native DNA or a PCR amplified product.

As used herein, the term “substantially identical”, when referring to a protein or polypeptide, is meant one that has at least 80%, 85%, 90%, 95%, or 99% sequence identity to a reference amino acid sequence. The length of comparison is preferably the full length of the polypeptide or protein, but is generally at least 10, 15, 20, 25, 30, 40, 50, 60, 80, or 100 or more contiguous amino acids. A “substantially identical” nucleic acid is one that has at least 80%, 85%, 90%, 95%, or 99% sequence identity to a reference nucleic acid sequence. The length of comparison is preferably the full length of the nucleic acid, but is generally at least 20 nucleotides, 30 nucleotides, 40 nucleotides, 50 nucleotides, 75 nucleotides, 100 nucleotides, 125 nucleotides, or more.

As used herein, the term “therapeutically effective amount” refers to a quantity of compound (e.g., a Otx2 protein or biologically active fragment thereof) delivered with sufficient frequency to provide a medical benefit to the patient. Thus, a therapeutically effective amount of a protein is an amount sufficient to treat or ameliorate a symptom of PD.

### BRIEF DESCRIPTION OF THE FIGURES

FIG. 1A-G show that Wnt1 directly regulates Lmx1a and Otx2 through the β-catenin complex. FIG. 1A is a schematic diagram showing that the two major signaling molecules involved in mDA differentiation are SHH from notochord and Wnt1 from Isthmus. FoxA2 is shown to be a direct downstream target of the SHH signaling pathway and then FoxA2 in turn induces VM SHH expression. FGF8 from the hindbrain side of Isthmus and Wnt1 from the midbrain side of Isthmus cross-regulate each other, shown by black arrow. FIG. 1B is a bar graph showing the qPCR analysis of DA regulator expression on in vitro differentiated cells transduced with empty or Wnt1-expressing retrovirus (ND3; n=4, p<0.05, data are represented as mean±SEM throughout this study). Wnt-1 overexpression results in a significant increase in the expression of Lmx1a, Otx2, and Pitx3. FIG. 1C-D are photomicrographs of LMX1a fluorescence immunocytochemistry on the same cells with nuclei stained using Hoechst dye. Scale bar represents 50 μm. FIG. 1E is a series of bar graphs showing the mRNA level of Gli1 in NP stage cells is increased following treatment with 500 ng/ml of SHH but reduced following treatment with μM Cyclopamine for 6 hours (left), whereas the Lmx1a levels were unchanged (right). FIG. 1F is a series of bar graphs showing the results of ChIP-qPCR analysis. In vitro differentiated cells were transduced with Wnt1-expressing retrovirus, treated with 15 mM LiCl for 24 hrs and fixed for ChIP at the NP stage. ChIP fragments were immunoprecipitated with normal rabbit IgG or anti-β-catenin antibody and analyzed by qPCR. The average of three independent ChIP analyses (n=3, p<0.05) are shown. FIG. 1G. is a series of bar graphs showing the results of ChIP using β-catenin antibody or IgG control (n=3, p<0.05) in E11.5 VMs, dissected as illustrated, without LiCl treatment.

FIG. 2A-D demonstrate that Lmx1a directly regulates Wnt1 expression. FIG. 2A is a bar graph showing, by qPCR analysis on in vitro differentiated ES cells, that Wnt1 expression but nto SHH or Wnt5a expression is increased following tranfection with an Lmx1a-expressing retrovirus (ND3; n=4, p<0.05). FIG. 2B-C are photomicrographs showing Wnt1 and Lmx1a immunocytochemistry on the same cells, respectively. Scale bar represents 50 μm. Inset shows Hoechst staining for nuclei. FIG. 2D is a series of bar graphs showing ChIP-qPCR analysis on Wnt1 and Wnt5a promoter region (n=3, p<0.05). In vitro differentiated ES cells transduced with retrovirus expressing HA-tagged Lmx1a was fixed for ChIP at ND3. ChIP fragments were immunoprecipitated either with normal rabbit IgG or anti-HA antibody and analyzed by qPCR. Results represent the average of three independent ChIP experiments.

FIG. 3A-M show that Lmx1a regulates Wnt1 expression during embryonic midbrain development. FIG. 3A-D is a series of photomicrographs showing in situ hybridization analysis of Wnt1 expression in coronal mesencephalic section of E10.5 (FIG. 3A, B) and E11.5 (FIG. 3C, D) littermate wt or dr/dr embryos. “d” marks dorsal mesencephalon and “v” marks VM. FIG. 3E-His a series of photomicrographs showing that Lmx1b is expressed in the entire ventral midbrain of E10.5 embryos but is restricted to the ventral most part in E11.5 embryos. Coronal midbrain sections were stained using Lmx1b or Lmx1a antibody. The white line marks ventricle. Scale bar represents 50 μm. FIG. 3I is a bar graph showing the results from dissected E11.5 VMs illustrated in the schematic, which were used for ChIP using Lmx1a antibody. FIG. 3J-K is a series of photomicrographs showing immunohistochemistry using an anti-corin antibody in E11.5 VM of littermate wt or dr/dr embryo. Scale bar represents 50 μm. FIG. 3L shows the FACS purification of mDA domain cells of littermate wt and dr/dr after staining with anti-corin antibody and Alexa-647-conjugated secondary antibody. The corin⁺ population is marked. FIG. 3M is a bar graph showing the results of a qPCR analysis of purified mDA domain cells on the expression of regulators of mDA neuronal development. The result is the average from three independent FACS purifications (n=3, p<0.05).

FIG. 4A-M show that Lmx1a directly regulates Nurr1 and Pitx3. A. qPCR analysis on in vitro differentiated ES cells with empty or Lmx1a-expressing retrovirus (ND3; n=4, p<0.05). B-C. Immunocytochemistry on the same cells. Scale bar represents 50 μm. D. ChIP-qPCR analysis on Nurr1 promoter region (n=3, p<0.05), performed as described above. E-F. Immunocytochemistry on the same cells. G. ChIP-qPCR analysis on Pitx3 promoter region (n=3, p<0.05), performed as described above. H-I. Immunohistochemistry analysis of VM in E12.5 littermates' wt and dr/dr embryos using anti-Nurr1 and anti-TH antibody. M denotes medial VM. Scale bar represents 50 μm. J. Cell counting analysis of Nurr1⁺ cells in ventral midbrain of E12.5 littermates' wt and dr/dr embryos (n=4, p<0.05). Cell numbers were counted from every 6^{th} sections using the Stereolnvestigator image capture equipment and software. The estimated total cell numbers based on counting every 6^{th} section are shown. K-L. Immunohistochemistry analysis of ventral midbrain in E12.5 littermates wt and dr/dr embryos using anti-Pitx3 antibody. M. Cell counting analysis of Pitx3⁺ cell numbers as described above (n=4, p<0.05).

FIG. 5A-P demonstrate the overlapping functions of Lmx1a and Lmx1b. A. qPCR analysis on in vitro differentiated cells transduced with empty, Lmx1a- or Lmx1b-expressing retrovirus at ND3 (n=4, p<0.05). O.E. denotes overexpression of Lmx1a (20.6±8.2). B. ChIP-qPCR analysis of Lmx1b (n=3, p<0.05). In vitro differentiated ES cells transduced with retrovirus expressing HA-tagged Lmx1b were fixed for ChIP at ND3. ChIP fragments were immunoprecipitated either with normal rabbit IgG or anti-HA antibody and analyzed by qPCR. Binding of Lmx1b to the Lmx1a target sites in the Wnt1, Nurr1 or Pitx3 promoters were tested using the same primer sets. C. qPCR analysis of siRNA-treated NP cells. ES cell-derived NP cells were treated with SHH and FGF8 for 4 days for induction/proliferation of mDA NPs and then transfected with control siRNA, Lmx1a siRNA, Lmx1b siRNA or Lmx1a/1b siRNAs, and analyzed 30 hours after transfection (n=4, p<0.05). D. qPCR analysis of siRNA-treated ND cells. ES cell-derived NP cells were treated with SHH and FGF8 for 4 days for induction/proliferation of mDA NPs, further differentiated until day 2 of ND stage, transfected with control siRNA, Lmx1a siRNA, Lmx1b siRNA or Lmx1a/1b siRNAs, and analyzed 30 hours after transfection (n=4, p<0.05). E-L. Immunocytochemistry on NP cells treated with control siRNA or Lmx1a/1b siRNAs one day after transfection. Scale bar represents 50 μm. M-P. Immunocytochemistry on ND cells treated with control siRNA or Lmx1a/1b siRNAs one day after transfection. Scale bar represents 50 μm.

FIG. 6A-W present that the Wnt1 signaling pathway induces mDA differentiation of ES cells synergistically with the SHH pathway. A. qPCR analysis on in vitro differentiated cells transduced with empty, FoxA2-, Lmx1a-, or Otx2-expressing retrovirus at ND6 (n=4, p<0.05). FLO designates cells transduced with all three viruses that express FoxA2, Lmx1a or Otx2. B-C. Co-transduction of three factors (FLO) leads to a significant increase in Pitx3⁺ TH⁺ mDA neurons compared to empty virus-transduced cells. D-G. Cell transduction with three factors does not significantly alter the proportion of neurons (Tuj1⁺) or astrocytes (GFAP⁺). H-O. Three factor transduction increases the cells with mature DA phenotype, shown by coexpression of DAT and DDC with TH. P-W. Three factor transduction increases cells with mDA phenotype, shown by coexpression of Lmx1b and Nurr1 with TH. Immunocytochemistry on in vitro differentiated ES cells at stage ND6. Inset shows Hoechst staining Scale bar represents 50 mm.

FIG. 7 illustrates the emerging genetic network of the Wnt1 signaling pathway that reveals the interaction between the Wnt1 and SHH pathways at three major steps of mDA development; i) ventralization and inhibition of alternate fates, ii) promotion of neurogenesis, and iii) DA phenotype specification and survival. Arrow indicates positive regulation and -| indicates negative regulation. Black arrows indicate the regulation previously shown. Gray arrows indicate the regulations observed in this study. Dotted lines represent regulations that are not shown to be direct yet. Solid lines represent regulation that has been shown to be direct.

FIG. 8 illustrates the overall scheme for in vitro differentiation of ES cells. ES cells were differentiated according to 5 stage procedure and transgene expressing vector were introduced either in ES cell stage (episomal vector) or NP stage (retroviral vector). Cells were analyzed at day 3 of ND stage.

FIG. 9 presents immunocytochemistry analysis of empty or Wnt1-retrovirus transduced cells at day 3 of ND stage, using antibody against Otx2(a-b), Pitx3(c-d) and Nurr1 (e-f). Scale bar=50 mm.

FIG. 10A-B show interaction between SHH and other factors. A Inhibition of SHH signaling does not interfere with regulation of Lmx1a by Wnt1. J1 ES-derived NP cells were transduced with empty- or Wnt1-retrovirus and cultured in the absence or presence of 1 mM cyclopamine for 3 days, and analyzed by qPCR at NP stage. B. Acute SHH treatment or Cyclopamine treatment did not alter mRNA expression of Nurr1 or TH. NP stage cells were treated with 500 ng/ml of SHH or 1 mM Cyclopamine for 6 hours and analyzed by qPCR.

FIG. 11 presents ChIP-qPCR analysis of β-catenin complex. Control in vitro differentiated ES cells or cells transduced with retrovirus expressing Wnt1 were fixed for ChIP at the NP stage without LiCl treatment. ChIP fragments were immunoprecipitated either with normal rabbit IgG or anti-b-catenin antibody. Enrichment of ChIP fragments by anti-b-catenin compared to control IgG were analyzed by qPCR (n=3, p<0.05, data are represented as mean±SEM).

FIG. 12A-C present Gel shift analysis results. A. Wnt1 probe specifically binds to Lmx1a. Radiolabled Wnt1 probe was incubated with NP nuclear extract and in the presence of normal sera or anti-Lmx1a sera. B. Nurr1 probe specifically binds to Lmx1a. Radiolabled Nurr1 probe was incubated with NP nuclear extract and in the presence of normal sera or anti-Lmx1a sera. C. Pitx3 probe specifically binds to Lmx1a. Radiolabled Pitx3 probe was incubated with NP nuclear extract and in the presence of normal sera or anti-Lmx1a sera. Black arrow indicates unshifted complex and gray arrow indicates supershifted complex by anti-Lmx1a antibody.

FIG. 13A-F demonstrate specificity of anti-Lmx1a antibody and anti-Lmx1b antibody. A-B. Immunocytochemistry on in vitro differentiated ES cells at day 3 of ND stage using anti-Lmx1a antibody and anti-Lmx1b antibody. There are many double positive cells, but also single positive cells, suggesting that anti-Lmx1a antibody does not cross-react with Lmx1b nor anti-Lmx1b antibody cross-react with Lmx1a. C-D. Lmx1b knockdown by siRNA does not alter Lmx1a+ cells. E-F. Lmx1a knockdown by siRNA does not alter Lmx1b+ cells. Scale bar=50 mm.

FIG. 14A-F show immunocytochemistry on in vitro differentiated ES cells. A-B. Immunocytochemistry on in vitro differentiated ES cells with Lmx1a episome at day 3 of ND stage using anti-Lmx1b antibody. Scale bar=50 mm C-F. Immunocytochemistry on in vitro differentiated ES cells with Lmx1a episome at day 7 of ND stage using anti-TH antibody and anti-Pitx3 antibody.

FIG. 15A-B show analysis of cell transduced with retrovirus expressing Lmx1a or Lmx1b. A. Retroviral Lmx1a expression increases endogenous Lmx1a expression, but, retroviral Lmx1b expression does not alter endogenous Lmx1b expression. qPCR analysis of endogenous mRNA expression level on in vitro differentiated cells transduced with empty, Lmx1a or Lmx1b-expressing retrovirus. Cells were harvested for RNA preparation at ND3. 3′UTR primers were used to detect only endogenous gene expression excluding mRNA expression from retroviral vectors. Fold changes in mRNA levels are shown where the value of empty vector controls being set as 1 (n=4, p<0.05, data are represented as mean±SEM). B. ChIP-qPCR analysis. In vitro differentiated ES cells were transduced with retrovirus expressing HA-tagged Lmx1a or HA-tagged Lmx1b, and fixed for ChIP at the ND stage. ChIP fragments were immunoprecipitated either with normal rabbit IgG or anti-HA antibody. Enrichment of ChIP fragments by anti-HA compared to control IgG were analyzed by qPCR (n=3, p<0.05, data are represented as mean±SEM). 10 kb upstream of the Lmx1a, Lmx1b or Ngn2 promoter regions were analyzed for well conserved homeodomain binding sites, whose the sequences are shown here.

FIG. 16 shows that the expression of FoxA2 and Otx2 is not affected by Lmx1a mutation. Immunohistochemistry analysis of ventral midbrain in E12.5 littermates wt and mutant embryos using anti-FoxA2 and anti-Otx2 antibody. Scale bar represents 50 mm.

FIG. 17 presents ChIP-qPCR analysis of cells transduced with Lmx1a or Lmx1b. In vitro differentiated ES cells were transduced with retrovirus expressing HA-tagged Lmx1a or HA-tagged Lmx1b, and fixed for ChIP at the ND stage. ChIP fragments were immunoprecipitated either with normal rabbit IgG or anti-HA antibody. Enrichment of ChIP fragments by anti-HA compared to control IgG were analyzed by qPCR (n=3, p<0.05, data are represented as mean±SEM). 10 kb upstream of Msx1 promoter regions were analyzed for well conserved homeodomain binding sites, and 18 well conserved sites contained in 8 independent PCR fragments are tested for Lmx1a or Lmx1b binding, and among them the sequence shown has the most significant binding.

FIG. 18 shows qPCR analysis of siRNA-treated NP cells. ES cell-derived NP cells were treated with SHH and FGF8 for 4 days for induction/proliferation of mDA NPs and then transfected with control siRNA, Lmx1a siRNA, Lmx1b siRNA or Lmx1a/1b siRNAs using Nucleofector (Amaxa), and analyzed 30 hours after transfection. Fold changes in mRNA levels are shown where the value of control siRNA-treated cells is set at 1 (n=4, p<0.05, data are represented as mean±SEM).

FIG. 19A-E show immunocytochemistry on cells transduced with FoxA2, Lmx1a or Otx2. A-B. Immunocytochemistry on in vitro differentiated ES cells with FoxA2, Lmx1a and Otx2 retrovirus transduction at day 6 of ND stage using anti-TH antibody and anti-Aldha1a antibody or anti-Calbindin antibody. C-E. Immunocytochemistry on in vitro differentiated ES cells with FoxA2, Lmx1a and Otx2 retrovirus transduction at day 6 of ND stage using anti-Tuj1 (b-tubulin) antibody and anti-5HT antibody, anti-ChAT antibody or anti-GABA antibody. Small insets show the hoechst staining Scale bar=50 mm.

FIG. 20A-B show that key TFs can be expressed in mammalian system. (A) Schematic representation of the mammalian expression vector of key TFs. The respective cDNAs of Nurr1, Pitx3, Lmx1a, FoxA2, and Otx2 were connected with a 9 arginine repeat, a myc tag, and a 6 histidine repeat. (B) Expression of Nurr1-9R and Pitx3-9R in HEK293 cells. Stable HEK293 cell lines were established that robustly express Nurr1-9R-myc-his (lane 2) and Pitx3-9R-myc-his (lane 4) fusion proteins, respectively. Cells were grown in the presence of G-418 to maintain stably transformed cells. Cell lysates (50 mg) from individual stable cell lines were subjected to SDS-PAGE followed by Western blotting using anti-mycantibodies. Lane 1 and 3 represent empty vector control cell lines.

FIG. 21 demonstrates that red fluorescent protein fused with 9R can penetrate all stage cells of mESC in vitro differentiation with almost 100% efficiency while naïve proteins can not. It also shows that even fully differentiated Tuj1+ neurons were transduced with 9R-dsRED almost completely.

FIG. 22A-D show that retroviral expression of Nurr1, Pitx3, or Lmx1a in mESC-derived NPs enhances their differentiation to DA neurons. (A) Experimental design of the 5 stage in vitro differentiation protocol of mESCs (ES, EB, NP selection, NP expansion, and DA neuron differentiation). (B) ESC-derived NPs were efficiently transduced by the retrovirus containing GFP and co-expressed with an NP marker, nestin. (C) Percentage of TH+ cells among total cells (DAPI+) per field for empty, Nurr1-, Pitx3-, and Lmx1a-transduced NP cells. Each of these factors enhanced DA neuron generation approximately 6-fold when SHH and FGF8 were treated for 2 days. The red bars indicate a limited treatment of SHH and FGF8, and blue bars indicate no treatment of SHH and FGF8. (D) Representative images of TH+ neuronal cells with DAPI staining.

FIG. 23 illustrates a proposed emerging regulatory network that indicates that the Wnt1 and the SHH signaling pathways co-operatively regulate DA neuron development by downstream key TFs; i) regionalization, inhibition of alternate fates, and promotion of neurogenesis by early factors (e.g., Lmx1a, Otx2, and FoxA2) and ii) DA phenotype specification and survival by late factors (e.g., Nurr1 and Pitx3). This regulatory network suggests that an optimal combination of these factors may synergistically induce differentiation of mESCs into mature DA neurons.

### DETAILED DESCRIPTION

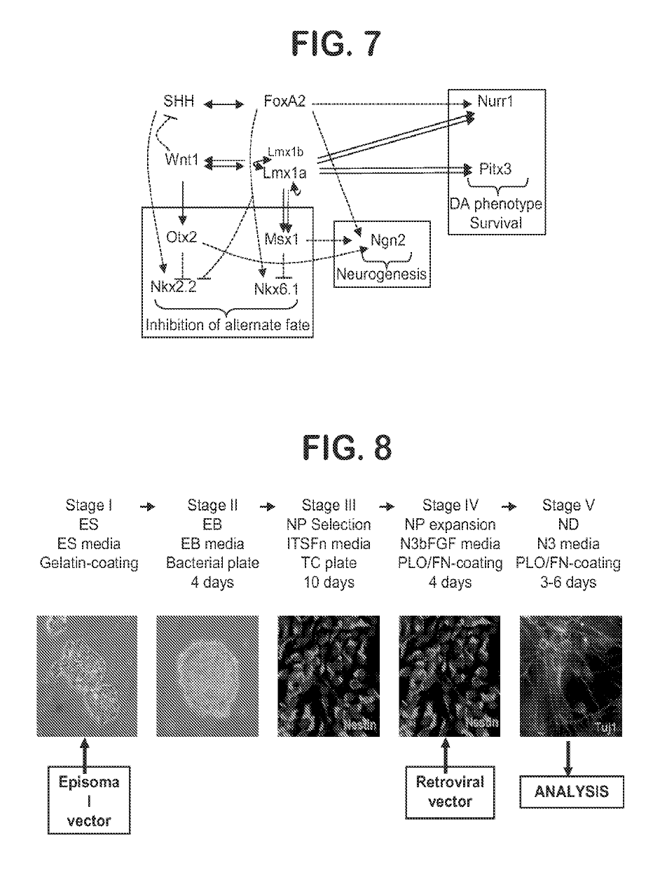

The present invention is based on the analysis of molecular networks involving Wnt1 during mDA differentiation of ES cells. It is shown that Wnt1 directly regulates Lmx1a, a key intrinsic factor for mDA differentiation, eliciting functional cascades that lead to mDA differentiation. The Wnt1-regulated molecular network described herein explains the functional role of Wnt1 in mDA phenotype specification apart from its well-established role in NP proliferation (Megason and McMahon, 2002). Furthermore, the extrinsic signaling molecule Wnt1 is identified as a major target of Lmx1a during mDA differentiation, forming an autoregulatory loop between Wnt1 and Lmx1a. It is further demonstrated that Lmx1a directly regulates two critical regulators of mDA neuron differentiation, the Nurr1 and Pitx3 genes as well as Wnt1 and that Wnt1 directly regulates Otx2 as well as Lmx1a through the canonical Wnt signaling pathway during mDA differentiation (FIG. 7). Further, the in vivo and in vitro analyses described herein show that the β-catenin complex indeed directly associates with the Lmx1a promoter and regulates its expression.

The finding that Pitx3 and Nurr1 are the direct downstream targets of the Wnt1-Lmx1a autoregulatory loop links a key signaling pathway of mDA differentiation to the major molecular regulators of terminal differentiation/survival of mDA neurons. In the experiments described herein, it is observed that Lmx1a directly binds to the Nurr1 promoter in vivo and activates Nurr1 expression. Thus, regulation of Nurr1 is one of the converging points of the SHH-FoxA2 pathway and the Wnt1-Lmx1a pathway. However, Lmx1a did not affect SHH or FoxA2 expression, showing the independent nature of these two pathways. In addition, the data show that Lmx1a is a link between a major signaling molecule Wnt1 and an important mDA-specific transcription factor, Pitx3.

As shown herein, Lmx1a and Lmx1b co-operatively regulate mDA neuron development by sharing redundant functions. First, the gene expression analyses of mDA domains and developing corin⁺ mFP cells showed that mDA phenotype is only mildly affected in Lmx1a mutant dr/dr embryo. Notably, the defect of target gene (Wnt1) expression was modest in the ventral most part where Lmx1b is still expressed, suggesting its compensating function. Second, the siRNA-based single and double knock down experiments of Lmx1a and Lmx1b in in vitro-differentiated ES cells showed that knocking down a single gene has no or marginal effect on target gene expression, whereas knocking down both genes significantly affected target gene expression. Third, the current extensive ChIP analyses indicate that both Lmx1a and Lmx1b directly bind to the promoters of target genes during mDA differentiation of ES cells, again supporting their redundant functions during mDA differentiation.

In summary, this invention is based on the discovery of an important complementary pathway for mDA development, the Wnt1-Lmx1a autoregulatory loop, during mDA differentiation of ES cells as well as during mouse embryonic midbrain development. Notably, this Wnt1-Lmx1a pathway is independent of the SHH-FoxA2 pathway, although they functionally interact with each other during mDA development. The data provided herein show that overexpression of SHH or its blocking by cyclopamine did not affect Lmx1a expression. In addition, induction of Lmx1a gene expression by Wnt1 during in vitro differentiation of mES cells was not affected by cyclopamine. That these two major signaling pathways, once formed, functionally interact with each other at three major steps of mDA development (FIG. 7). The functional interactions between these two pathways predict that activation of key mediators of both signaling pathways may facilitate ES cell differentiation to mDA neurons by efficiently providing the proper cellular environment for each other. Activation of both pathways by exogenous expression of three key mediators resulted in synergistic induction of mDA differentiation, compared to the induction of a single pathway. The invention demonstrates the usefulness of ES cell differentiation to investigate the molecular network of mDA differentiation and also in turn, show that the invention can facilitate the generation of cell sources for cell replacement therapy for PD.

It has been discovered that activation of both the Wnt1-Lmx1a and the SHH-FoxA2 signaling pathways can synergistically induce mDA differentiation and inhibit differentiation into other neural cell types and therefore effectively produce a mDA population of high purity. Activation of the Wnt1-Lmx1a signaling pathway can be achieved by, for example, increasing the biological activity of one or more proteins selected from the group consisting of Wnt1, Lmx1a, Lmx1b, Otx2 and Pitx3. Activation of the SHH-FoxA2 signaling pathway can be achieved by, for example, increasing the biological activity of one or more proteins from the group consisting of SHH, FoxA2 and Nurr1.

Accordingly, in one embodiment, the Wnt1-Lmx1a and the SHH-FoxA2 signaling pathways can be activated by increasing the biological activity of FoxA2, Lmx1a and/or Otx2, or alternatively increasing the biological activity of Nurr1, Pitx3 and/or Lmx1a, or alternatively increasing the biological activity of Nurr1, Pitx3, Lmx1a, FoxA2 and/or Otx2.

Additionally, activation of the Wnt1-Lmx1a and/or the SHH-FoxA2 signaling pathways can achieved by increasing the biological activities of proteins that interact directly or indirectly with these signaling pathways, such as, but not limited to, En1, En2 and Ngn2

Methods of increasing the biological activity of a gene or protein are known in the art and are further described below.

### Methods for Increasing the Level of a Protein in a Cell

Methods for increasing the level of a protein, or polypeptide or peptide, in a cell are known in the art. In one aspect, the protein level is increased by increasing the amount of a polynucleotide encoding the protein, wherein that polynucleotide is expressed such that new protein is produced. In another aspect, increasing the protein level is increased by increasing the transcription of a polynucleotide encoding the protein, or alternatively translation of the protein, or alternatively post-translational modification, activation or appropriate folding of the protein. In yet another aspect, increasing the protein level is increased by increasing the binding of the protein to appropriate cofactor, receptor, activator, ligand, or any molecule that is involved in the protein's biological functioning. In some embodiments, increasing the binding of the protein to the appropriate molecule is increasing the amount of the molecule. In one aspect of the embodiments, the molecule is a protein. In another aspect of the embodiments, the molecule is a small molecule. In a further aspect of the embodiments, the molecule is a polynucleotide.

Methods of increasing the amount of polynucleotide encoding the protein in a cell are known in the art. In one aspect, the polynucleotide can be introduced to the cell and expressed by a gene delivery vehicle that can include a suitable expression vector.

Suitable expression vectors are well-known in the art, and include vectors capable of expressing a polynucleotide operatively linked to a regulatory element, such as a promoter region and/or an enhancer that is capable of regulating expression of such DNA. Thus, an expression vector refers to a recombinant DNA or RNA construct, such as a plasmid, a phage, recombinant virus or other vector that, upon introduction into an appropriate host cell, results in expression of the inserted DNA. Appropriate expression vectors include those that are replicable in eukaryotic cells and/or prokaryotic cells and those that remain episomal or those which integrate into the host cell genome.

As used herein, the term “vector” refers to a non-chromosomal nucleic acid comprising an intact replicon such that the vector may be replicated when placed within a cell, for example by a process of transformation. Vectors may be viral or non-viral. Viral vectors include retroviruses, adenoviruses, herpesvirus, papovirus, or otherwise modified naturally occurring viruses. Exemplary non-viral vectors for delivering nucleic acid include naked DNA; DNA complexed with cationic lipids, alone or in combination with cationic polymers; anionic and cationic liposomes; DNA-protein complexes and particles comprising DNA condensed with cationic polymers such as heterogeneous polylysine, defined-length oligopeptides, and polyethylene imine, in some cases contained in liposomes; and the use of ternary complexes comprising a virus and polylysine-DNA.

Non-viral vector may include plasmid that comprises a heterologous polynucleotide capable of being delivered to a target cell, either in vitro, in vivo or ex-vivo. The heterologous polynucleotide can comprise a sequence of interest and can be operably linked to one or more regulatory elements and may control the transcription of the nucleic acid sequence of interest. As used herein, a vector need not be capable of replication in the ultimate target cell or subject. The term vector may include expression vector and cloning vector.

A “viral vector” is defined as a recombinantly produced virus or viral particle that comprises a polynucleotide to be delivered into a host cell, either in vivo, ex vivo or in vitro. Examples of viral vectors include retroviral vectors, adenovirus vectors, adeno-associated virus vectors, alphavirus vectors and the like. Alphavirus vectors, such as Semliki Forest virus-based vectors and Sindbis virus-based vectors, have also been developed for use in gene therapy and immunotherapy. See, Schlesinger and Dubensky (1999) Curr. Opin. Biotechnol. 5:434-439 and Ying, et al. (1999) Nat. Med. 5(7):823-827. In aspects where gene transfer is mediated by a retroviral vector, a vector construct refers to the polynucleotide comprising the retroviral genome or part thereof, and a therapeutic gene. As used herein, “retroviral mediated gene transfer” or “retroviral transduction” carries the same meaning and refers to the process by which a gene or nucleic acid sequences are stably transferred into the host cell by virtue of the virus entering the cell and integrating its genome into the host cell genome. The virus can enter the host cell via its normal mechanism of infection or be modified such that it binds to a different host cell surface receptor or ligand to enter the cell. As used herein, retroviral vector refers to a viral particle capable of introducing exogenous nucleic acid into a cell through a viral or viral-like entry mechanism.

Retroviruses carry their genetic information in the form of RNA; however, once the virus infects a cell, the RNA is reverse-transcribed into the DNA form which integrates into the genomic DNA of the infected cell. The integrated DNA form is called a provirus.

In aspects where gene transfer is mediated by a DNA viral vector, such as an adenovirus (Ad) or adeno-associated virus (AAV), a vector construct refers to the polynucleotide comprising the viral genome or part thereof, and a transgene. Adenoviruses (Ads) are a relatively well characterized, homogenous group of viruses, including over 50 serotypes. See, e.g., International PCT Application No. WO 95/27071. Ads do not require integration into the host cell genome. Recombinant Ad derived vectors, particularly those that reduce the potential for recombination and generation of wild-type virus, have also been constructed. See, International PCT Application Nos. WO 95/00655 and WO 95/11984. Wild-type AAV has high infectivity and specificity integrating into the host cell's genome. See, Hermonat and Muzyczka (1984) Proc. Natl. Acad. Sci. USA 81:6466-6470 and Lebkowski et al. (1988) Mol. Cell. Biol. 8:3988-3996.

Vectors that contain both a promoter and a cloning site into which a polynucleotide can be operatively linked are well known in the art. Such vectors are capable of transcribing RNA in vitro or in vivo, and are commercially available from sources such as Stratagene (La Jolla, Calif.) and Promega Biotech (Madison, Wis.). In order to optimize expression and/or in vitro transcription, it may be necessary to remove, add or alter 5′ and/or 3′ untranslated portions of the clones to eliminate extra, potential inappropriate alternative translation initiation codons or other sequences that may interfere with or reduce expression, either at the level of transcription or translation. Alternatively, consensus ribosome binding sites can be inserted immediately 5′ of the start codon to enhance expression.

Gene delivery vehicles also include DNA/liposome complexes, micelles and targeted viral protein-DNA complexes. Liposomes that also comprise a targeting antibody or fragment thereof can be used in the methods of this invention. To enhance delivery to a cell, the nucleic acid or proteins of this invention can be conjugated to antibodies or binding fragments thereof which bind cell surface antigens, e.g., a cell surface marker found on stem cells or cardiomyocytes. In addition to the delivery of polynucleotides to a cell or cell population, direct introduction of the proteins described herein to the cell or cell population can be done by the non-limiting technique of protein transfection, alternatively culturing conditions that can enhance the expression and/or promote the activity of the proteins of this invention are other non-limiting techniques.

Proteins have been described that have the ability to translocate desired nucleic acids across a cell membrane. Typically, such proteins have amphiphilic or hydrophobic subsequences that have the ability to act as membrane-translocating carriers. For example, homeodomain proteins have the ability to translocate across cell membranes. The shortest internalizable peptide of a homeodomain protein, Antennapedia, was found to be the third helix of the protein, from amino acid position 43 to 58 (see, e.g., Prochiantz, 1996, Current Opinion in Neurobiology 6:629-634. Another subsequence, the h (hydrophobic) domain of signal peptides, was found to have similar cell membrane translocation characteristics (see, e.g., Lin et al., 1995, J. Biol. Chem. 270:14255-14258). Such subsequences can be used to translocate oligonucleotides across a cell membrane. Oligonucleotides can be conveniently derivatized with such sequences. For example, a linker can be used to link the oligonucleotides and the translocation sequence. Any suitable linker can be used, e.g., a peptide linker or any other suitable chemical linker.

Methods of delivering a protein to a cell, either to increase the biological activity of itself or a protein positively regulated by this protein, or to decrease the biological activity of a protein negatively regulated by this protein, are generally known in the art. For example, proteins can be delivered to a eukarotic cell by a type III sercreation machine. See, e.g., Galan and Wolf-Watz (2006) Nature 444:567-73. Biologically active and full length protein, for another example, can also be delivered into a cell using cell penetraint peptides (CPP) as delivery vehicles. The trans-activating transcriptional activator (TAT) from human immunodeficiency virus 1 (HIV-1) is such a CPP, which is able to deliver different proteins, such as horseradish peroxidase and RNase A across cell membrane into the cytoplasm in different cell lines. Wadia et al. (2004) Nat. Med. 10:310-15. Accordingly, in one aspect, a protein, such as Lmx1b, can be delivered to a neural precursor cell using TAT as a vehicle to increase the biological activity of Lmx1b in the cell.

Liposomes, microparticles and nanoparticles are also known to be able to facilitate delivery of proteins or peptides to a cell (reviewed in Tan et al., (2009) Peptides 2009 Oct. 9. [Epub ahead of print]). The liposomes, microparticles or nanoparticles can also comprise a targeting antibody or fragment thereof can be used in the methods of this invention. To enhance delivery to a cell, the proteins can be conjugated to antibodies or binding fragments thereof which bind cell surface antigens, e.g., a cell surface marker found on progentior cells.

In another aspect, non-covalent method which forms CPP/protein complexes has also been developed to address the limitations in covalent method such as chemical modification before crosslinking and denaturation of proteins before delivery. For example, a short amphipathic peptide carrier, Pep-1 and protein complexes have proven effective for delivery. It was shown that Pep-1 could facilitate rapid cellular uptake of various peptides, proteins and even full-length antibodies with high efficiency and less toxicity. Cheng et al. (2001) Nat. Biotechnol. 19:1173-6.

Proteins can be synthesized for delivery. Nucleic acids that encode a protein or fragment thereof may be introduced into various cell types or cell-free systems for expression, thereby allowing purification of the Wnt1, Lmx1a, and/or Lmx1b, or other proteins, for large-scale production and patient therapy.

Eukaryotic and prokaryotic expression systems may be generated in which a gene sequence is introduced into a plasmid or other vector, which is then used to transform living cells. Constructs in which the cDNA contains the entire open reading frame inserted in the correct orientation into an expression plasmid may be used for protein expression. Prokaryotic and eukaryotic expression systems allow for the protein to be recovered, if desired, as fusion proteins or further containing a label useful for detection and/or purification of the protein. Typical expression vectors contain regulatory elements that direct the synthesis of large amounts of mRNA corresponding to the inserted nucleic acid in the plasmid-bearing cells. They may also include a eukaryotic or prokaryotic origin of replication sequence allowing for their autonomous replication within the host organism, sequences that encode genetic traits that allow vector-containing cells to be selected for in the presence of otherwise toxic drugs, and sequences that increase the efficiency with which the synthesized mRNA is translated. Stable long-term vectors may be maintained as freely replicating entities by using regulatory elements of, for example, viruses (e.g., the OriP sequences from the Epstein Barr Virus genome). Cell lines may also be produced that have integrated the vector into the genomic DNA, and in this manner the gene product is produced on a continuous basis.

Expression of foreign sequences in bacteria, such as Escherichia coli, requires the insertion of the nucleic acid sequence into a bacterial expression vector. Such plasmid vectors contain several elements required for the propagation of the plasmid in bacteria, and for expression of the DNA inserted into the plasmid. Propagation of only plasmid-bearing bacteria is achieved by introducing, into the plasmid, selectable marker-encoding sequences that allow plasmid-bearing bacteria to grow in the presence of otherwise toxic drugs. The plasmid also contains a transcriptional promoter capable of producing large amounts of mRNA from the cloned gene. Such promoters may be (but are not necessarily) inducible promoters that initiate transcription upon induction. The plasmid also preferably contains a polylinker to simplify insertion of the gene in the correct orientation within the vector.

Stable or transient cell line clones of mammalian cells can also be used to express a protein. Appropriate cell lines include, for example, COS, HEK293T, CHO, or NIH cell lines.

Once the appropriate expression vectors containing a gene, fragment, fusion, or mutant are constructed, they are introduced into an appropriate host cell by transformation techniques, such as, but not limited to, calcium phosphate transfection, DEAE-dextran transfection, electroporation, microinjection, protoplast fusion, or liposome-mediated transfection. The host cells that are transfected with the vectors of this invention may include (but are not limited to) E. coli or other bacteria, yeast, fungi, insect cells (using, for example, baculoviral vectors for expression in SF9 insect cells), or cells derived from mice, humans, or other animals (e.g., mammals). In vitro expression of a protein, fusion, polypeptide fragment, or mutant encoded by cloned DNA may also be used. Those skilled in the art of molecular biology will understand that a wide variety of expression systems and purification systems may be used to produce recombinant proteins and fragments thereof.

Once a recombinant protein is expressed, it can be isolated from cell lysates using protein purification techniques such as affinity chromatography. Once isolated, the recombinant protein can, if desired, be purified further by e.g., by high performance liquid chromatography (HPLC; e.g., see Fisher, Laboratory Techniques In Biochemistry And Molecular Biology, Work and Burdon, Eds., Elsevier, 1980).

The term “antibody” refers to one or more polyclonal antibodies, monoclonal antibodies, antibody compositions, antibodies having mono- or poly-specificity, humanized antibodies, single-chain antibodies, chimeric antibodies, CDR-grafted antibodies, antibody fragments such as Fab, Fab′, F(ab)₂, Fv, and other antibody fragments which retain the antigen binding function of the parent antibody. Antibodies may be raised against any portion of a protein which provides an antigenic epitope. Methods to make and use antibodies to inhibit protein function are described in e.g., U.S. Pat. No. 7,320,789 and U.S. Patent Application Publication No. 2009/0010929.

### Vectors Suitable for Delivery to Humans

This invention features methods and compositions for treating or preventing PD. In one aspect, the invention features methods of gene therapy to express Otx2, Lmx1a, Lmx1b, FoxA1, FoxA2 or other proteins in the midbrain, suitably in the dopaminergic neurons of the midbrain, of a patient. Gene therapy, including the use of viral vectors as described herein, seeks to transfer new genetic material (e.g., polynucleotides encoding Otx2, Lmx1a, Lmx1b, FoxA1, FoxA2 or other proteins or a biologically active fragment thereof) to the cells of a patient with resulting therapeutic benefit to the patient. For in vivo gene therapy, expression vectors encoding the gene of interest is administered directly to the patient. The vectors are taken up by the target cells (e.g., neurons or pluripotent stem cells) and the gene expressed. Recent reviews discussing methods and compositions for use in gene therapy include Eck et al., in Goodman & Gilman's The Pharmacological Basis of Therapeutics, Ninth Edition, Hardman et al., eds., McGray-Hill, New York, 1996, Chapter 5, pp. 77-101; Wilson, Clin. Exp. Immunol. 107 (Suppl. 1):31-32, 1997; Wivel et al., Hematology/Oncology Clinics of North America, Gene Therapy, S. L. Eck, ed., 12(3):483-501, 1998; Romano et al., Stem Cells, 18:19-39, 2000, and the references cited therein. U.S. Pat. No. 6,080,728 also provides a discussion of a wide variety of gene delivery methods and compositions.

Adenoviruses are able to transfect a wide variety of cell types, including non-dividing cells. There are more than 50 serotypes of adenoviruses that are known in the art, but the most commonly used serotypes for gene therapy are type 2 and type 5. Typically, these viruses are replication-defective; and genetically-modified to prevent unintended spread of the virus. This is normally achieved through the deletion of the E1 region, deletion of the E1 region along with deletion of either the E2 or E4 region, or deletion of the entire adenovirus genome except the cis-acting inverted terminal repeats and a packaging signal (Gardlik et al., Med Sci Monit. 11: RA110-121, 2005).

Retroviruses are also useful as gene therapy vectors and usually (with the exception of lentiviruses) are not capable of transfecting non-dividing cells. Accordingly, any appropriate type of retrovirus that is known in the art may be used, including, but not limited to, HIV, SIV, FIV, EIAV, and Moloney Murine Leukaemia Virus (MoMLV). Typically, therapeutically useful retroviruses including deletions of the gag, pol, or env genes.

In another aspect, the invention features the methods of gene therapy that utilize a lentivirus vectors to express Wnt1, Lmx1a, and/or Lmx1b, or other proteins in a patient. Lentiviruses are a type of retroviruses with the ability to infect both proliferating and quiescent cells. An exemplary lentivirus vector for use in gene therapy is the HIV-1 lentivirus. Previously constructed genetic modifications of lentiviruses include the deletion of all protein encoding genes except those of the gag, pol, and rev genes (Moreau-Gaudry et al., Blood. 98: 2664-2672, 2001).

Adeno-associated virus (AAV) vectors can achieve latent infection of a broad range of cell types, exhibiting the desired characteristic of persistent expression of a therapeutic gene in a patient. The invention includes the use of any appropriate type of adeno-associated virus known in the art including, but not limited to AAV1, AAV2, AAV3, AAV4, AAV5, and AAV6 (Lee et al., Biochem J. 387: 1-15, 2005; U.S. Patent Publication 2006/0204519).

Herpes simplex virus (HSV) replicates in epithelial cells, but is able to stay in a latent state in non-dividing cells such as the midbrain dopaminergic neurons. The gene of interest may be inserted into the LAT region of HSV, which is expressed during latency. Other viruses that have been shown to be useful in gene therapy include parainfluenza viruses, poxviruses, and alphaviruses, including Semliki forest virus, Sinbis virus, and Venezuelan equine encephalitis virus (Kennedy, Brain. 120: 1245-1259, 1997).

Exemplary non-viral vectors for delivering nucleic acid include naked DNA; DNA complexed with cationic lipids, alone or in combination with cationic polymers; anionic and cationic liposomes; DNA-protein complexes and particles comprising DNA condensed with cationic polymers such as heterogeneous polylysine, defined-length oligopeptides, and polyethylene imine, in some cases contained in liposomes; and the use of ternary complexes comprising a virus and polylysine-DNA. In vivo DNA-mediated gene transfer into a variety of different target sites has been studied extensively. Naked DNA may be administered using an injection, a gene gun, or electroporation. Naked DNA can provide long-term expression in muscle. See Wolff, et al., Human Mol. Genet., 1:363-369, 1992; Wolff, et al., Science, 247, 1465-1468, 1990. DNA-mediated gene transfer has also been characterized in liver, heart, lung, brain and endothelial cells. See Zhu, et al., Science, 261: 209-211, 1993; Nabel, et al., Science, 244:1342-1344, 1989. DNA for gene transfer also may be used in association with various cationic lipids, polycations and other conjugating substances. See Przybylska et al., J. Gene Med., 6: 85-92, 2004; Svahn, et al., J. Gene Med., 6: S36-S44, 2004.

Methods of gene therapy using cationic liposomes are also well known in the art. Exemplary cationic liposomes for use in this invention are DOTMA, DOPE, DOSPA, DOTAP, DC-Chol, Lipid GL-67™, and EDMPC. These liposomes may be used in vivo or ex vivo to encapsulate a Otx2 vector for delivery into target cells (e.g., neurons or pluripotent stem cells).

Typically, vectors made in accordance with the principles of this disclosure will contain regulatory elements that will cause constitutive expression of the coding sequence. Desirably, neuron-specific regulatory elements such as neuron-specific promoters are used in order to limit or eliminate ectopic gene expression in the event that the vector is incorporated into cells outside of the target region. Several regulatory elements are well known in the art to direct neuronal specific gene expression including, for example, the neural-specific enolase (NSE), and synapsin-1 promoters (Morelli et al. J. Gen. Virol. 80: 571-583, 1999).

### Direct Protein Administration

The level of a protein also may be increased in cells by directly administering that protein to the cells in a manner in which the protein is taken up by the cell (i.e., transits across the cell membrane into the cytoplasm). To help facilitate the delivery of any protein into a cell and across the cell membrane, the protein may be fused chemically or recombinantly, or otherwise associated with a peptide that facilitates the delivery, such as a cell penetrating peptides (CPP) or protein transduction domain (PTD).

Cell penetrating peptides, or “CPPs”, as used herein, refer to short peptides that facilitate cellular uptake of various molecular cargos (from small chemical molecules to nanosize particles and large fragments of DNA). A “cargo”, such as a protein, is associated with the peptides either through chemical linkage via covalent bonds or through non-covalent interactions. The function of the CPPs are to deliver the cargo into cells, a process that commonly occurs through endocytosis with the cargo delivered to the endosomes of living mammalian cells. CPPs typically have an amino acid composition containing either a high relative abundance of positively charged amino acids such as lysine or arginine, or have sequences that contain an alternating pattern of polar/charged amino acids and non-polar, hydrophobic amino acids. In 1988, Frankel and Pabo found that the human immunodeficiency virus transactivator of transcription (HIV-TAT) protein can be delivered to cells using a CPP (Frankel et al., 1988a and Frankel et al., 1988b).

A CPP employed in accordance with one aspect of the invention may include 3 to 35 amino acids, preferably 5 to 25 amino acids, more preferably 10 to 25 amino acids, or even more preferably 15 to 25 amino acids.

A CPP may also be chemically modified, such as prenylated near the C-terminus of the CPP. Prenylation is a post-translation modification resulting in the addition of a 15 (farneysyl) or 20 (geranylgeranyl) carbon isoprenoid chain on the peptide. A chemically modified CPP can be even shorter and still possess the cell penetrating property. Accordingly, a CPP, pursuant to another aspect of the invention, is a chemically modified CPP with 2 to 35 amino acids, preferably 5 to 25 amino acids, more preferably 10 to 25 amino acids, or even more preferably 15 to 25 amino acids.

A CPP suitable for carrying out one aspect of the invention may include at least one basic amino acid such as arginine, lysine and histidine. In another aspect, the CPP may include more, such as 2, 3, 4, 5, 6, 7, 8, 9, 10, or more such basic amino acids, or alternatively about 5%, 10%, 15%, 20%, 25%, 30%, 40%, 50% of the amino acids are basic amino acids. In one embodiment, the CPP contains at least two consecutive basic amino acids, or alternatively at least three, or at least five consecutive basic amino acids. In a particular aspect, the CPP includes at least two, three, four, or five consecutive arginine. In a further aspect, the CPP includes more arginine than lysine or histidine, or preferably includes more arginine than lysine and histidine combined.

CPPs may include acidic amino acids but the number of acidic amino acids should be smaller than the number of basic amino acids. In one embodiment, the CPP includes at most one acidic amino acid. In a preferred embodiment, the CPP does not include acidic amino acid. In a particular embodiment, a suitable CPP is the HIV-TAT peptide.

CPPs can be linked to a protein recombinantly, covalently or non-covalently. A recombinant protein having a CPP peptide can be prepared in bacteria, such as E. coli, a mammalian cell such as a human HEK293 cell, or any cell suitable for protein expression. Covalent and non-covalent methods have also been developed to form CPP/protein complexes. A CPP, Pep-1, has been shown to form a protein complex and proven effective for delivery (Kameyama et al., 2006 Bioconjugate Chem. 17:597-602).

CPPs also include cationic conjugates which also may be used to facilitate delivery of the proteins into the progenitor or stem cell. Cationic conjugates may include a plurality of residues including amines, guanidines, amidines, N-containing heterocycles, or combinations thereof. In related embodiments, the cationic conjugate may comprise a plurality of reactive units selected from the group consisting of alpha-amino acids, beta-amino acids, gamma-amino acids, cationically functionalized monosaccharides, cationically functionalized ethylene glycols, ethylene imines, substituted ethylene imines, N-substituted spermine, N-substituted spermidine, and combinations thereof. The cationic conjugate also may be an oligomer including an oligopeptide, oligoamide, cationically functionalized oligoether, cationically functionalized oligosaccharide, oligoamine, oligoethyleneimine, and the like, as well as combinations thereof. The oligomers may be oligopeptides where amino acid residues of the oligopeptide are capable of forming positive charges. The oligopeptides may contain 5 to 25 amino acids; preferably 5 to 15 amino acids; more preferably 5 to 10 cationic amino acids or other cationic subunits.

Recombinant proteins anchoring CPP to the proteins can be generated to be used for delivery to neural progenitor cells or stem cells to prepare mature and functional DA neurons.

Accordingly, in one aspect, the invention provides a method for producing a neural cell from neural progenitor cells or stem cells by contacting a neural progenitor cell or neural stem cell with at least one protein of the Wnt1-Lmx1a signaling pathway selected from the group consisting of Wnt1, Lmx1b, Lmx1b, Otx2 and Pitx3 and at least one protein of the SHH-FoxA2 signaling pathway selected from the group consisting of SHH, FoxA2 and Nurr1 under conditions suitable for the proteins to penetrate the cells. Preferably, each of the proteins is attached to a CPP. In some embodiments, the proteins comprise FoxA2, Lmx1a and/or Otx2, or alternatively include Nurr1, Pitx3 and/or Lmx1a, or alternatively include Nurr1, Pitx3, Lmx1a, FoxA2 and/or Otx2. In some embodiment, the neural cells can be further in contact with one or more of En1, En2 and/or Ngn2, which can be optionally attached to a CPP.

The neural progenitor cell or stem cell can be embryonic stem cells or cell lines, induced pluripotent stem cells or adult stem cell.

### Pharmaceutical or Therapeutic Compositions

The invention, in another aspect, provides a neural cell or cell population produced by the methods of the invention as disclosed herein. The neural cell, in one aspect, is a mDA neural cell. The neural cell, in another aspect, expresses tyrosine hydroxylase, or alternatively further expresses at least one of Pitx3, Nurr1, dopamine transporter (DAT), and dopa decarboxylase (DDC). In yet another aspect, the invention provides a pharmaceutical composition comprising a neural cell produced by the methods of the invention and a pharmaceutically acceptable carrier or excipient.

The present invention also includes the administration of therapeutic molecules, such as polynucleotides, proteins or small molecules to a subject. The therapeutic molecules can be administered to a subject, e.g., a human, alone or in combination with any pharmaceutically acceptable carrier or salt known in the art. Pharmaceutically acceptable salts may include non-toxic acid addition salts or metal complexes that are commonly used in the pharmaceutical industry. Examples of acid addition salts include organic acids such as acetic, lactic, pamoic, maleic, citric, malic, ascorbic, succinic, benzoic, palmitic, suberic, salicylic, tartaric, methanesulfonic, toluenesulfonic, or trifluoroacetic acids or the like; polymeric acids such as tannic acid, carboxymethyl cellulose, or the like; and inorganic acids such as hydrochloric acid, hydrobromic acid, sulfuric acid phosphoric acid, or the like. Metal complexes include zinc, iron, and the like. Exemplary pharmaceutically acceptable carriers include physiological saline and artificial cerebrospinal fluid (aCSF). Other physiologically acceptable carriers and their formulations are known to one skilled in the art and described, for example, in Remington: The Science and Practice of Pharmacy, (21st edition), 2005, Lippincott Williams & Wilkins Publishing.

Pharmaceutical formulations of a therapeutically effective amount of a compound of the invention, or pharmaceutically acceptable salt-thereof, can be administered parenterally (e.g. intramuscular, intraperitoneal, intravenous, or subcutaneous injection), or by intrathecal or intracerebroventricular injection in an admixture with a pharmaceutically acceptable carrier adapted for the route of administration.

Formulations for parenteral administration include sterile aqueous or non-aqueous solutions, suspensions, or emulsions. Examples of suitable vehicles include propylene glycol, polyethylene glycol, vegetable oils, gelatin, hydrogenated naphalenes, and injectable organic esters, such as ethyl oleate. Such formulations may also contain adjuvants, such as preserving, wetting, emulsifying, and dispersing agents. Biocompatible, biodegradable lactide polymer, lactide/glycolide copolymer, or polyoxyethylene-polyoxypropylene copolymers may be used to control the release of the compounds. Other potentially useful parenteral delivery systems for the proteins of the invention include ethylene-vinyl acetate copolymer particles, osmotic pumps, implantable infusion systems, and liposomes.

Liquid formulations can be sterilized by, for example, filtration through a bacteria-retaining filter, by incorporating sterilizing agents into the compositions, or by irradiating or heating the compositions. Alternatively, they can also be manufactured in the form of sterile, solid compositions which can be dissolved in sterile water or some other sterile injectable medium immediately before use.

The protein or therapeutic compound can be administered in a sustained release composition, such as those described in, for example, U.S. Pat. No. 5,672,659 and U.S. Pat. No. 5,595,760. The use of immediate or sustained release compositions depends on the type of condition being treated. If the condition consists of an acute or subacute disorder, a treatment with an immediate release form will be preferred over a prolonged release composition. Alternatively, for preventative or long-term treatments, a sustained released composition will generally be preferred.

### Transplantation of Neural or Progenitor Cells

In another aspect, ex vivo gene therapy is used to effect gene expression in the midbrain of a patient. Generally, this therapeutic strategy involves using the expression vectors and techniques described above to transfect cultured cells in vitro prior to implantation of those cells into the brain (i.e., the midbrain) of a patient. The advantage of this strategy is that the clinician can ensure that the cultured cells are expressing suitable levels of genes in a stable and predictable manner prior to implantation. Such preliminary characterization also allows for more precise control over the final dosage of proteins that will be expressed by the modified cells.

In one embodiment, autologous cells are isolated, transfected, and implanted into the patient. The use of autologous cells minimizes the likelihood of rejection or other deleterious immunological host reaction. Other useful cell types include, for example, pluripotent stem cells, including umbilical cord blood stem cells, neuronal progenitor cells, fetal mesencephalic cells, embryonic stem cells, and postpartum derived cells (U.S. Patent Application 2006/0233766). In another embodiment, cells are encapsulated in a semipermeable, microporous membrane and transplanted into the patient adjacent to the substantia nigra (WO 97/44065 and U.S. Pat. Nos. 6,027,721; 5,653,975; 5,639,275), the caudate, and/or the putamen. The encapsulated cells are modified to express a secreted version of encoded proteins in order to provide a therapeutic benefit to the surrounding brain regions. The secreted proteins may be native proteins, biologically active protein fragments, and/or modified proteins which have increased cell permeability relative to the native proteins (e.g., proteins fused to a CPP).

Cell transplantation therapies typically involve grafting the replacement cell populations into the lesioned region of the nervous system (e.g., the A9 region of the substantia nigra, the caudate, and/or the putamen), or at a site adjacent to the site of injury. Most commonly, the therapeutic cells are delivered to a specific site by stereotaxic injection. Conventional techniques for grafting are described, for example, in Bjorklund et al. (Neural Grafting in the Mammalian CNS, eds. Elsevier, pp 169-178, 1985), Leksell et al. (Acta Neurochir., 52:1-7, 1980) and Leksell et al. (J. Neurosurg., 66:626-629, 1987). Identification and localization of the injection target regions will generally be done using a non-invasive brain imaging technique (e.g., MRI) prior to implantation (see, for example, Leksell et al., J. Neurol. Neurosurg. Psychiatry, 48:14-18, 1985).

Briefly, administration of cells into selected regions of a patient's brain may be made by drilling a hole and piercing the dura to permit the needle of a microsyringe to be inserted. Alternatively, the cells can be injected into the brain ventricles or intrathecally into a spinal cord region. The cell preparation permits grafting of the cells to any predetermined site in the brain or spinal cord. It also is possible to effect multiple grafting concurrently, at several sites, using the same cell suspension, as well as mixtures of cells. Multiple graftings may be unilateral, bilateral, or both. Typically, grafting into larger brain structures such as the caudate and/or putamen will require multiple graftings at spatially distinct locations.

Following in vitro cell culture and isolation as described herein, the cells are prepared for implantation. The cells are suspended in a physiologically compatible carrier, such as cell culture medium (e.g., Eagle's minimal essential media), phosphate buffered saline, or artificial cerebrospinal fluid (aCSF). Cell density is generally about 107 to about 108 cells/ml. The volume of cell suspension to be implanted will vary depending on the site of implantation, treatment goal, and cell density in the solution. For the treatment of Parkinson's Disease, about 30-100 μl of cell suspension will be administered in each intra-nigral or intra-putamenal injection and each patient may receive a single or multiple injections into each of the left and right nigral or putaminal regions.

In some embodiments, the cells expressing Wnt1, Lmx1a, and/or Lmx1b or other proteins are encapsulated within permeable membranes prior to implantation. Encapsulation provides a barrier to the host's immune system and inhibits graft rejection and inflammation. Several methods of cell encapsulation may be employed. In some instances, cells will be individually encapsulated. In other instances, many cells will be encapsulated within the same membrane. Several methods of cell encapsulation are well known in the art, such as described in European Patent Publication No. 301,777, or U.S. Pat. Nos. 4,353,888, 4,744,933, 4,749,620, 4,814,274, 5,084,350, and 5,089,272.

In one method of cell encapsulation, the isolated cells are mixed with sodium alginate and extruded into calcium chloride so as to form gel beads or droplets. The gel beads are incubated with a high molecular weight (e.g., MW 60-500 kDa) concentration (0.03-0.1% w/v) polyamino acid (e.g., poly-L-lysine) to form a membrane. The interior of the formed capsule is re-liquefied using sodium citrate. This creates a single membrane around the cells that is highly permeable to relatively large molecules (MW ˜200-400 kDa), but retains the cells inside. The capsules are incubated in physiologically compatible carrier for several hours in order that the entrapped sodium alginate diffuses out and the capsules expand to an equilibrium state. The resulting alginate-depleted capsules is reacted with a low molecular weight polyamino acid which reduces the membrane permeability (MW cut-off ˜40-80 kDa).

### Identification of Candidate Compounds Useful for Treating or Preventing Parkinson's Disease

A candidate compound that is beneficial for treating or preventing PD can be identified using the methods described herein. A candidate compound can be identified for its ability to increase the expression or biological activity of at least one of Otx2, Lmx1a, and FoxA2 protein. Candidate compounds that modulate the expression level or biological activity of the protein by at least 10%, 20%, 30%, 40%, 50%, 60%, 70%, 80%, 90%, 95%, 100%, or more relative to an untreated control not contacted with the candidate compound are identified as compounds useful for treating and preventing PD.

A wide array of cell types, may be used in the screening methods of this invention to identify candidate compounds for the treatment of PD by assessing the effects of the candidate compounds on the expression of at least one of Otx2, Lmx1a, and FoxA2 protein. Primary fetal dopaminergic neurons or cell lines exhibiting some characteristics of the dopaminergic neuronal phenotype may be used in the present invention. Cell lines have the advantage of providing a homogeneous cell population, which allows for reproducibility and sufficient number of cells for experiments. Primary dopaminergic cultures are derived from tissues harvested from developing ventral mesencephalon (VM) containing the substantia nigra. They have the advantage of containing authentic dopaminergic neurons cultured in a context of their naturally occurring neighboring cells. Cell lines, including immortalized cell lines, may be used for screening candidate compounds. Preferably, the cell lines adopt a neuronal phenotype, such as a dopaminergic neuronal phenotype. Suitable cell lines include, for example, Human Dopaminergic Neuron Precursor (DAN) cells and PC-12 cells.

### Kits

In a further aspect, the invention disclosure provides kits for treating PD. The kits can comprise a therapeutic molecule, a pharmaceutical composition or a neuronal or progenitor cell as disclosed here for the use to treat or prevent PD and instructions to use.

### EXAMPLES

### Example 1

### Experimental Procedures

1.1 Plasmid Construction and Retroviral Preparation

The episomal expression vectors were constructed by inserting the PCR-amplified coding region of mouse Lmx1a cDNA into the Xho I and Not I sites of the pPyCAGIP vector (Chambers et al., 2003). The correct cDNA insertion into the vector was confirmed by restriction digestion and sequence analyses. Retroviral expression vectors were constructed by inserting the PCR-amplified coding region of mouse Lmx1a, mouse Lmx1b, human FoxA2, human Otx2 or mouse Wnt1 into the XhoI and NotI sites of the pCL vector. For the HA-tagged Lmx1a or HA-tagged Lmx1b retroviral construct, the coding region of mouse Lmx1a or mouse Lmx1b fused in frame with the HA-tag at the C-terminus was PCR-amplified and inserted into the pCL vector. Retrovirus was prepared using the 293GPG retroviral producer cell line as described in Ory et al. (Ory et al., 1996).

1.2 ES Cell Culture and In Vitro Differentiation

ES cells were maintained and differentiated as described previously (Chung et al., 2002).

For transgenic expression studies, suboptimal conditions were intentionally used to clearly see the effect of transgene expression without masking its effect by stimulating its upstream events by culture conditions. So neither any signaling molecules such as SHH, FGF8, Ascorbic Acids nor any growth factors such GDNF nor BDNF, nor any feeders such as MS5 nor PA6 was added (Andersson et al., 2006b; Kawasaki et al., 2000; Kim et al., 2002).

For siRNA transfection, NP stage cells were treated with 50 ng/ml FGF8 and 100 ng/ml SHH for 4 days to induce/proliferate mDA NPs, followed by transfection with siRNA using the Nucleofector (Amaxa, Walkersville, Md.) mouse stem cell kit with the program A-033 according to the manufacturer's instruction. Per transfection, 5×10⁶ NP cells were treated with 480 pmol of siRNA, diluted in 10 ml of N3bFGF media (or N3 media for ND stage transfection) and plated in PLO/FN-coated multiwell plate, resulting in a final siRNA concentration of 48 nM. For ND stage transfection, cells were further differentiated in N3 media for 2 days before transfection. Multiple Stealth siRNAs were purchased from Invitrogen (Carlsbad, Calif.), screened for gene silencing efficiency by cotransfection with Lmx1a or Lmx1b-expressing plasmids and only siRNAs showing efficient gene silencing (>95%) was used for the experiments. The sequences of the siRNAs are as follows: the Lmx1a sense strand GAGGAGAGCAUUCAAGGCCUCGUUU (SEQ ID NO: 1); the Lmx1a antisense strand AAACGAGGCCUUGAAUGCUCUCCUC (SEQ ID NO: 2); the Lmx1b sense strand GGAACGACUCCAUCUUCCACGAUAU (SEQ ID NO: 3); the Lmx1b antisense strand AUAUCGUGGAAGAUGGAGUCGUUCC (SEQ ID NO: 4). Thirty hours after transfection, cells were fixed for immunocytochemistry or harvested for RNA preparation.

The mouse blastocyst-derived ES cell line J1 was a kind gift from Dr. Jaenisch, and was propagated and maintained as described previously (Chung et al., 2002). Briefly, undifferentiated ES cells were cultured on gelatin-coated dishes in Dulbecco's modified Minimal Essential Medium (Invitrogen, Carlsbad, Calif.) supplemented with 2 mM glutamine (Invitrogen, Carlsbad, Calif.), 0.001% β-mercaptoethanol (Invitrogen, Carlsbad, Calif.), 1× non-essential amino acids (Invitrogen, Carlsbad, Calif.), 10% donor horse serum (Sigma, St. Louis, Mo.), and 2000 U/ml human recombinant leukemia inhibitory factor (LIF; R & D Systems, Minneapolis, Minn.).

ES cells were differentiated into embryoid bodies (EBs) on nonadherent bacterial dishes (Fisher Scientific, Pittsburgh, Pa.) for four days in LIF-free EB medium containing 10% fetal bovine serum (Hyclone, Logan, Utah) instead of horse serum. EBs were then plated onto adhesive tissue culture surface (Fisher Scientific, Pittsburgh, Pa.). After 24 hrs in culture, selection of neuronal precursor cells was initiated in serum-free ITSFn medium. After 10 days of selection, cells were trypsinized and nestin, neuronal precursors were plated on polyornithine (15 μg/ml; Sigma, St. Louis, Mo.) and fibronectin (1 μg/ml; Sigma, St. Louis, Mo.) coated coverslips in N2 medium supplemented with 1 μg/ml laminin (Sigma, St. Louis, Mo.) and 10 ng/ml bFGF (R & D Systems, Minneapolis, Minn.) (Neuronal precursor expansion stage: NP stage). After expansion for four days, bFGF was removed to induce differentiation to neuronal phenotypes (Neuronal differentiation stage: ND stage). Cells were eventually fixed in 4% paraformaldehyde or harvested in TriReagent (Sigma, St. Louis, Mo.) at ND3.

For stable transfection of ES cells, J1 cells were transfected with polyoma large T antigen-expressing construct pMGDneo (Gassmann et al., 1995) to increase the stable transfection efficiency of episomal constructs (Gassmann et al., 1995) and stable cells were selected in the presence of 500 μg/ml G418. The resultant J1MGD cells were further transfected with episomal vectors using lipofectamine 2000 (Invitrogen, Carlsbad, Calif.) according to the manufacturer's instruction. Stably transfected episomal cells were selected in ES medium containing 500 μg/ml G418 and 1 μg/ml puromycin. For retroviral transfection of ES-derived NPs, J1 cells were differentiated to NP stage and infected with retrovirus (MOI=4) for 3 hrs in the presence of 2 μg/ml polybrene, which resulted in >90% infection. After 3 more days' expansion in N3bFGF media, cells were finally differentiated in N3 media and fixed at ND3.

1.3 Cell Counting and Statistical Analysis

Cells were counted from blind-coded samples using an integrated Axioskop 2 microscope (Carl Zeiss, Thornwood, N.Y.) and the StereoInvestigator image capture equipment and software (Microbright Field, Williston, Vt.). For statistical analysis, the Statview software was used and analysis of variance (ANOVA) was performed with an alpha level of 0.05 to determine possible statistical differences between group means. When significant differences were found, post hoc analysis was performed using Fisher's PLSD (alpha=0.05).

1.4 Immunocytochemistry & Immunohistochemistry

For immunofluorescence staining, cells were fixed in 4% formaldehyde (Electron Microscopy Sciences, Ft. Washington, Pa.) for 30 minutes, rinsed with PBS and then incubated with blocking buffer (PBS, 10% normal donkey serum; NDS) for 10 minutes. Cells were then incubated overnight at 4° C. with primary antibodies diluted in PBS containing 2% NDS. The following primary antibodies were used: rabbit-anti-corin (a kind gift from Dr. Morgan; 1:1,000), rabbit anti-Lmx1a (a kind gift from Dr. German; 1:1,000), ginea pig anti-Lmx1b (a kind gift from Dr. Birchmeier; 1:10,000), mouse anti-BrdU (Invitrogen, Carlsbad, Calif.; 1:100), rabbit anti-Nurr1 (Santa Cruz, Santa Cruz, Calif.; 1:200), rabbit anti-Pitx3 (Invitrogen, Carlsbad, Calif.; 1:200), rabbit anti-FoxA2 (Abcam, Cambridge, Mass.; 1:1,000), goat anti-Otx2 (Neuromics, Edina, Minn.; 1:2,000), rabbit anti-β-tubulin (Covance; 1:2000), rabbit-GFAP(DAKO, Carpinteria, Calif.; 1:1,000), sheep anti-tyrosine hydroxylase (TH) (Pel-Freez, Rogers, Arkansas; 1:500), Rabbit anti-Wnt1 (ABR, Golden, Colo.; 1:200), rat anti-DAT (Chemicon, Billerica, Mass.; 1:1,000), rabbit anti-Aldh1al (a kind gift from Dr. Duester; 1:1,000), rabbit anti-calbindin (Swant, Switzerland; 1:10,000), rabbit anti-5HT (Sigma, St. Louis, Mo.; 1:1,000), rabbit anti-ChAT (Chemicon, Billerica, Mass.; 1:200) and anti-GABA (Sigma, St. Louis, Mo.; 1:1,000). After additional rinsing in PBS, samples were incubated in fluorescent-labeled secondary antibodies (Alexa 488- or Alexa 594-labeled IgG; Invitrogen, Carlsbad, Calif.) in PBS with 2% NDS for 30 minutes at room temperature. After rinsing in PBS, Hoechst 33342 (4 [symbol] g/ml) was used for counterstaining, and coverslips/tissues sections were mounted onto slides in Mowiol 4-88 (Calbiochem, Gibbstown, N.J.). Confocal analysis was performed using a Zeiss LSM510/Meta Station (Carl Zeiss, Thornwood, N.Y.).

1.5 qPCR Analysis

For RNA preparation from FACS-purified cells, the RNeasy Micro kit (Qiagen, Valencia, Calif.) was used, and cDNA was prepared using Message Booster cDNA synthesis kit (Epicentre Biotechnologies, Madison, Wis.) according to the manufacturer's instruction.

For all other samples, total RNA was prepared using TriReagent (Sigma, St. Louis, Mo.) followed by further purification using RNeasy mini kit (Qiagen, Valencia, Calif.). For RT-PCR analysis, 2 [symbol] g of RNA were transcribed into cDNA with the SuperScript™ II RT (Invitrogen, Carlsbad, Calif.) and oligo (dT) primers. For quantitative analysis of the expression level of mRNAs, real-time PCR analyses using SYBR green I were performed using a DNA engine Opticon™ (MJ Research, Waltham, Mass.). To reduce non-specific signals, oligonucleotides amplifying small amplicons were designed using the MacVector software (Oxford Molecular Ltd.: primers sequences are available upon request). Amplifications were performed in 25 μl containing 0.5 μM of each primer,

0.5×SYBR Green I (Molecular Probes, Oreg.), and 2 μl of 5 fold diluted cDNA. Fifty PCR cycles were performed with a temperature profile consisting of 95° C. for 30 sec, 55° C. for 30 sec, 72° C. for 30 sec, and 79° C. for 5 sec. The dissociation curve of each PCR product was determined to ensure that the observed fluorescent signals were only from specific PCR products. After each PCR cycle, the fluorescent signals were detected at 79° C. to melt primer dimers (Tms of all primer dimers used in this study were <76° C.). A standard curve was constructed using plasmid DNAs containing the GAPDH gene (from 10² to 10⁸ molecules). The fluorescent signals from specific PCR products were normalized against that of the β-actin gene, and then relative values were calculated by setting the normalized value of control as 1 (or 100 in some cases). All reactions were repeated using more than three independent samples.

Ten μm Embryo cryosections were fixed for 10 min in 4% paraformaldehyde, washed in PBT, and permeabilized with 1.0 μg/mL proteinase K for 10 min. Sections were then washed in PBS, fixed in 4% paraformaldehyde for 5 min, and acetylated in 0.25% acetic anhydride in 0.1 M triethanolamine for 10 min, washed in PBS, rinsed in water, and air dried for 30 min. RNA probe was then added in 100 μl of hybridization buffer (10 mM Tris pH 7.5, 600 mM NaCl, 1 mM EDTA, 0.25% SDS, 10% Dextran Sulfate, 1×Denhardt's, 200 μg/mL yeast tRNA, 50% formamide). Slides were covered with coverslips cut from polypropylene bags, placed in chambers humidified with 1×SSC/50% formamide, and incubated overnight at 65° C. The next day, coverslips were removed with 5×SSC and slides were washed for 30 min in 1×SSC/50% formamide at 65° C. Slides were then transferred to TNE (10 mM Tris pH 7.5, 500 mM NaCl, 1 mM EDTA) at 37° C. for 10 min, incubated in RNase A (20 μg/mL, Roche, Indianapolis, Ind.) in TNE for 30 min at 37° C., and then washed in TNE for 10 min. Sections were then washed in 2×SSC for 20 min at 65° C., then washed twice for 20 min each in 0.2×SSC, and then transferred into MABT. Slides were blocked in 2% blocking reagent (Roche, Indianapolis, Ind.)/20% heat inactivated goat serum/MABT for 1 h. Secondary antibody was added (anti-DIG AP antibody, 1:2000, Roche, Indianapolis, Ind.) in 2% blocking reagent/20% heat inactivated sheep serum/MABT, and slides were incubated overnight at 4° C. The next day, slides were washed in MABT and equilibrated in NTM for 10 min. Color detection was performed using BCIP/NBT. The sequences of primers used for amplifying probes are available upon request.

1.6 ChIP-qPCR Analysis

ChIP-qPCR analysis was done as described previously (Yochum et al., 2007). For Lmx1a or Lmx1b ChIP, HA-tagged Lmx1a-expressing retrovirus or HAtagged Lmx1b-expressing retrovirus was transduced into J1 cells at the NP stage (MOI=4) and further differentiated until ND3. Cells were fixed and chromatin was immunoprecipitated using a ChIPAssay kit (Upstate, Billerica, Mass.) according to the manufacturer's instructions. Briefly, after fixation in 1% PFA for 10 minutes at 37° C., chromatin was sheared with a Sonic Dismembrator (Fisher Scientific, output setting 2.5, ten 20-sec pulses with 30-sec incubation on ice between pulses) to a size of 100 bp to 700 bp as verified on a 1% agarose gel. Clarified nuclear lysates were incubated overnight at 4° C. with a rabbit anti-HA polyclonal antibody (Abcam, Cambridge, Mass.) or rabbit normal IgG as a control. Following this incubation, Protein A agarose beads were added and allowed to bind for 60 min at 4° C. Immunoprecipitates were washed and crosslinks were reversed for 4 hours at 65° C. ChIP DNA was purified by incubation with RNase A for 1 h at 37° C., with 200 μg/ml Proteinase K for 2 h at 45° C., phenol:chloroform:isoamyl alcohol extraction, and precipitation with 0.1 volumes of 3M sodium acetate, 2.5 volumes of 100% ethanol and 1 μl of glycogen as a carrier. For β-catenin ChIP, Wnt1-expressing retrovirus was transduced into J1 cells at the NP stage (MOI=4) and further differentiated until day 4 of the NP stage. In some cases, cells were treated with 15 mM LiCl for 24 hrs before fixation to stabilize β-catenin, as indicated in the figure legend. Fixing of the cells and immunoprecipitation of Chromatin was as described above except five 20-sec pulses were used instead of ten pulses for sonication. For immunoprecipitation, mouse anti-β-catenin antibody (BD Transduction, Lexington, Ky.) together with rabbit anti-mouse IgG (Jackson ImmunoResearch, West Grove, Pa.) were used. For β-catenin ChIP or Lmx1a ChIP from mouse embryonic ventral midbrain, timed pregnant mice (C57BL/6) were purchased from Charles River laboratory (Wilmington, Mass.). The ventral mesencephalon from E11.5 embryos was dissected, fixed and processed for ChIP as described above except that, instead of protein A-sepharose beads, protein A-dyna beads (Invitrogen, Carlsbad, Calif.) were used as described in (Dahl and Collas, 2008). Minimal amount (10 [symbol] l) of dyna beads were used for each ChIP to decrease the high nonspecific background caused by small input of material. Lmx1a antibody is a kind gift from Dr. German (UCSF).

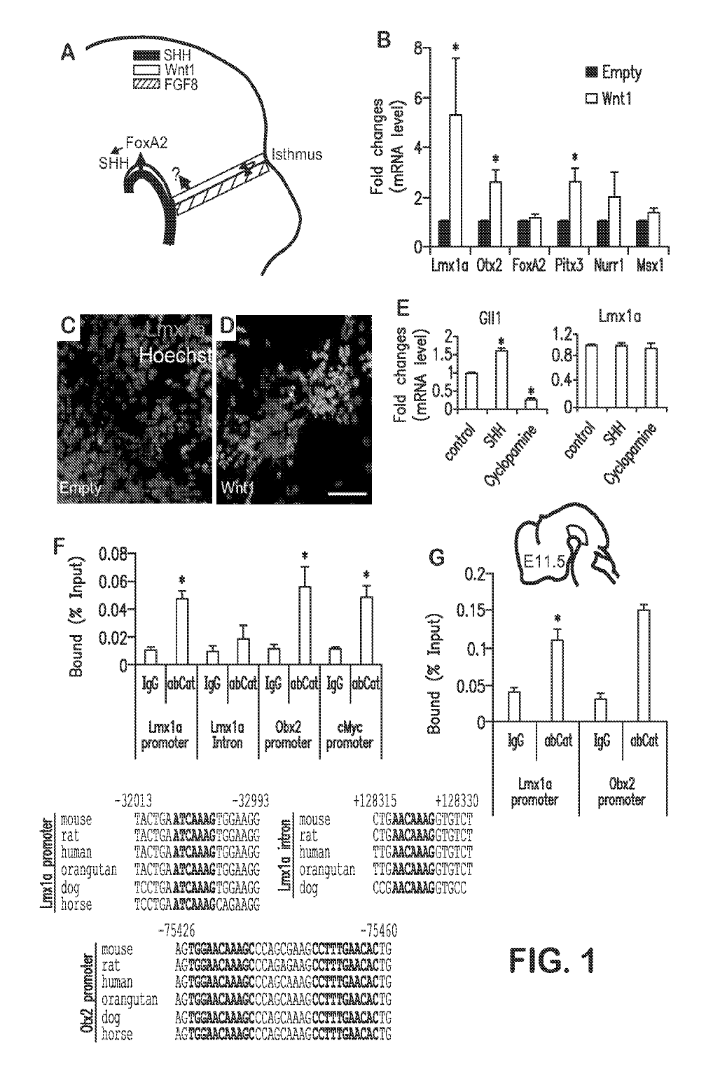

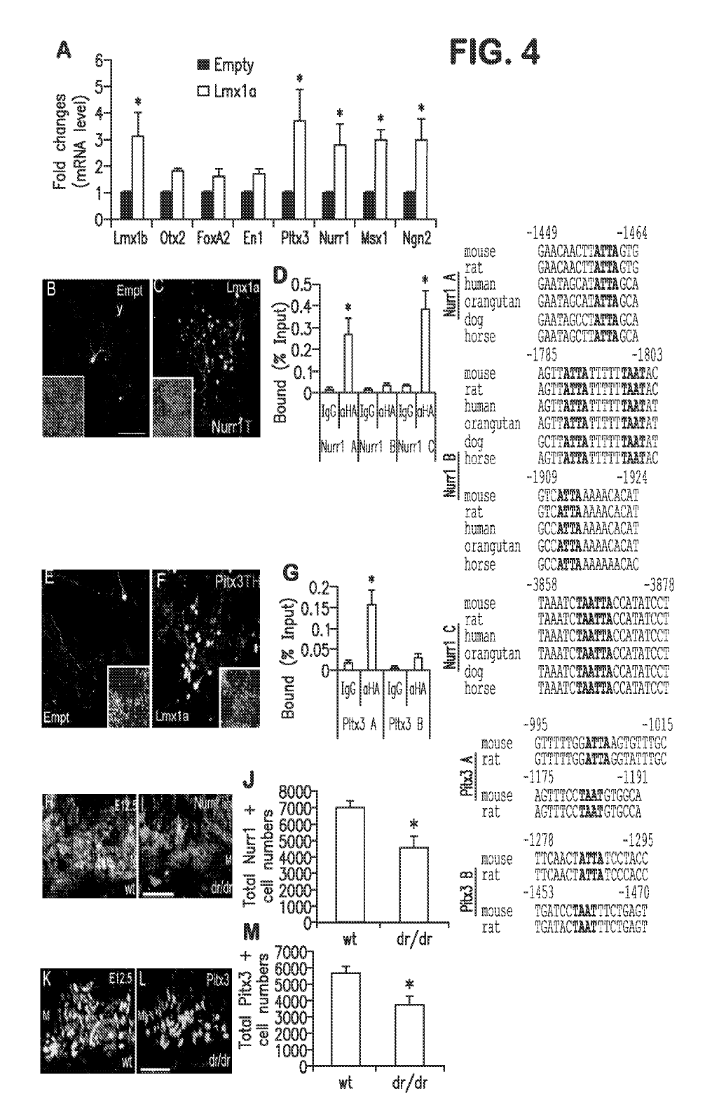

For the qPCR analysis of ChIP fragments, ECR (evolutionally conserved region) in the promoter region of each gene were analyzed with the ECR browser (www.ecrbrowser.dcode.org) and well-conserved binding sites within ECR were identified. For potential Lmx1a binding site(s) in the Wnt1 promoter, there is a start site for another gene 12 kb upstream of the Wnt1 gene promoter, so the promoter region up to 6 kb was analyzed and only 1 well-conserved homeodomain-binding site was identified in this region. For well-conserved homeodomain binding site in the Nurr1 promoter, there are numerous well-conserved homeodomain-binding sites in the 10 kb upstream region of the Nurr1 gene promoter (77 sites total). Four well-conserved homeodomain sites residing in the proximal 2 kb promoter and on regions of multiple homeodomain binding site clusters up to 5 kb of the promoter were focused on. For the Pitx3 promoter, up to 2 kb of its upstream promoter was analyzed, because there is another gene 4 kb upstream of the Pitx3 start site. Four homeodomain-binding sites, conserved between mouse and rat, have been identified in this region (FIG. 4G), which were contained in two separate PCR fragments, Pitx3 A and B. For well-conserved TCF/LEF binding sites in the Lmx1a promoter, the Lmx1a promoter region up to 40 kb was analyzed for evolutionarily conserved regions (ECR) and well-conserved TCF/LEF binding sites and identified a single well-conserved TCF/LEF binding site (FIG. 1F). For well-conserved TCF/LEF binding sites in the Otx2 promoter, a 1.4 kb forebrain and midbrain-specific enhancer has been identified at −75 kb in the upstream promoter region with potential TCF/LEF binding sites (Kurokawa et al., 2004).

PCR primer pairs were designed to amplify genomic fragments containing well conserved binding sites using the MacVector software (Oxford Molecular Ltd.). Real-time PCR was carried out with SYBR green as described above. Samples from three independent ChIP assays were analyzed for each candidate target sites.

1.7 Murine Models

Heterozygous dreher mice were purchased from the Jackson Laboratory (Bar Harbor, Me.). B6C3Fe a/a-Lmx1adr-J/J mouse harbors a point mutation, which makes Lmx1a protein non-functional (Millonig et al., 2000).

Embryos were genotyped by PCR amplification using the following primer sets followed by restriction digestion of PCR product using HpyCH4V (New England Biolabs, Ipswich, Mass.).

(SEQ ID NO: 5)
Forward primer: 5′GGAGACCACCTGCTTCTACC3′
(SEQ ID NO: 6)
Reverse primer: 5′GCATACGGATGGACTTCCC3′

Plug date was considered as embryonic day (E) 0.5. Embryos were fixed by immersion in 4% paraformaldehyde, equilibrated in sucrose (20% in PBS), sectioned at 10 μm on a cryostat, and collected onto glass slides. For each experiment, at least three sets of littermate wt and mutant pairs were used.

1.8 FACS Purification

FACS purification was done as described previously (Pruszak et al., 2007).

Timed pregnant mice were sacrificed and E11.5 embryos were harvested and genotyped. Ventral mesencephalon of littermate wt and mutant embryos was dissected, and the rostral to isthmus, pooled and gently triturated. Cells were filtered through cell strainer caps (35 μm mesh) to obtain a single cell suspension (5×10₆ cells/ml for sorting). Corin₊ cells were labeled by incubating with an anti-corin antibody (a kind gift from Dr. Morgan) for 30 min at 4° C., followed by incubation for 20 to 30 min with Alexafluor-647-conjugated anti-rabbit antibodies (Invitrogen, Carlsbad, Calif.). All washing steps were performed in phenol-free, Ca₊₊, Mg₊₊-free Hank's buffered saline solution (HBSS; Invitrogen, Carlsbad, Calif.) containing Penicillin-Streptomycin, 20 mM D-Glucose and 2% fetal bovine serum. Stained cells were sorted on a fluorescenceactivated cell sorter FACSAria (BD Biosciences, San Jose, Calif.) using FACSDiva software (BD Biosciences, San Jose, Calif.). The population of interest, excluding debris and dead cells, was identified by forward and side scatter gating. Corin positivity was determined compared to negative controls lacking the primary antibody and lacking primary and secondary antibodies. Sorts were repeated three times. Prior to sorting, the nozzle, sheath and sample lines were sterilized with 70% ethanol or 2% hydrogen peroxide for 15 min, followed by washes with sterile water to remove remaining decontaminant. A 100-μm ceramic nozzle (BD Biosciences), sheath pressure of 20 to 25 PSI and an acquisition rate of 1,000 to 3,000 events per second were used (“gentle FACS”) (Pruszak et al., 2007).

1.9 EMSA (Electrophoretic Mobility Shift Assay)

Nuclear extracts were prepared from Lmx1a-transfected 293T cells using Nuclear Extraction kit (Panomics) according to manufacturer's instruction. Sense (S) and antisense (A) oligonucleotides sequences are as follows.

Wnt1 promoter Lmx1a binding site-S
(SEQ ID NO: 7)
CTATGAGTGCACGTCGTTAATCACAAACACACACA
Wnt1 promoter Lmx1a binding site-A
(SEQ ID NO: 8)
GTGTGTGTGTTTGTGATTAACGACGTGCACTCATA
Nurr1 promoter Lmx1a binding site-S
(SEQ ID NO: 9)
GTGATAAGAAATAAATCTAATTACCATATCCTTTGAATA
Nurr1 promoter Lmx1a binding site-A
(SEQ ID NO: 10)
CTATTCAAAGGATATGGTAATTAGATTTATTTCTTATCA
Pitx3 promoter Lmx1a binding site-S
(SEQ ID NO: 11)
CTTGCAAACACTTAATCCAAAAACGCCAA
Pitx3 promoter Lmx1a binding site-A
(SEQ ID NO: 12)
GTTGGCGTTTTTGGATTAAGTGTTTGCAA

The sense and antisense oligonucleotides were annealed, gel-purified, and ³²P-labeled by T4 DNA kinase and used as probes in electrophoretic mobility shift assays (EMSA). EMSA and antibody coincubation experiments were performed using 30,000-50,000 cpm of labeled probe (˜0.05-0.1 ng) and nuclear extracts (10-30 μg) in a final volume of 20 μl of 12.5% glycerol, and (in mM) 12.5 HEPES, pH 7.9, 4 Tris-HCl, pH 7.9, 60 KCl, 1 EDTA, and 1 DTT with 1 μg of poly(dI-dC). For supershift assay, antibody was coincubated with the nuclear extract mix for 30 min at 4° C. before adding the radiolabeled probe. Antibody against Lmx1a was kindly provided from Dr. M. German (UCSF).

1.10 Sequences for ChIP-qPCR Analysis

(SEQ ID NO: 13)
TCF-lmx1apromoterF AAGAGTTCAGAAGGCAACCCTGGCTC
(SEQ ID NO: 14)
TCF-lmx1apromoterR CAGACCCTCCCCGATTTATTTC
(SEQ ID NO: 15)
TCF-otxpromoterF TGTTCAAAGGCTTCGCTGGG
(SEQ ID NO: 16)
TCF-otxpromoterR ACACACACACACACACAAAACTTCAG
(SEQ ID NO: 17)
TCF-lmx1aIntronF ATCCTGGCACAGATCCTCCTTC
(SEQ ID NO: 18)
TCF-lmx1aIntronR CCAATCAGCTCCTAGAGTCTCAGAATC
(SEQ ID NO: 19)
TCF-cmycpromoterF GGAGAGGGTTTGAGAGGGAGCA
(SEQ ID NO: 20)
TCF-cmycpromoterR TGGGGAAGGTGGGGAGGAGA
(SEQ ID NO: 21)
HD-wnt1F TGACTCAAGGTGCCATAGGGTG
(SEQ ID NO: 22)
HD-wnt1R GTGTGTGTGTGTGTGTGTGTGTTTG
(SEQ ID NO: 23)
HD-wnt5aAF TTGTTCGGGTCTGAAGGACAGG
(SEQ ID NO: 24)
HD-wnt5aAR GCAAGATTTCCAGCAAGCGTATC
(SEQ ID NO: 25)
HD-wnt5aBF TGATTTATTTGTTCCTCGGAGTCC
(SEQ ID NO: 26)
HD-wnt5aBR GCGTGTGCTCTCTGATTAAGTCATC
(SEQ ID NO: 27)
HD-wnt5aCF AAGAGGTAAACATCTTGGGGCG
(SEQ ID NO: 28)
HD-wnt5aCR GGTTGTCATTCTGGGAAAATCTACC
(SEQ ID NO: 29)
HD-wnt5aDF AAATGCCCCCTAACCTCAAGGGAG
(SEQ ID NO: 30)
HD-wnt5aDR TGGAATACTAGAAAAGGGACAAAGAGG
(SEQ ID NO: 31)
HD-nurrAF CTCTCACTTTTTCCCTTTCGTTCG
(SEQ ID NO: 32)
HD-nurrAR AGTTTCCCCCAGATGTTGCG
(SEQ ID NO: 33)
HD-nurrBF TGTGCTTGCTCCTTGGTGTTC
(SEQ ID NO: 34)
HD-nurrBR CTATCTTCAGACAGTCGGAAACCC
(SEQ ID NO: 35)
HD-NurrCF TTGATTAGCTCCCTCCCCAGTC
(SEQ ID NO: 36)
HD_nurrCR CACTACAAAAGTAACTTATAGCAAGGACCC
(SEQ ID NO: 37)
HD-pitxAF AAGGGGGGATTGGAAGTTCAGGTC
(SEQ ID NO: 38)
HD-pitxAR GGGTAGCGTTGGCGTTTTTG
(SEQ ID NO: 39)
HD-ptxBF CTCAGATAAAACAGAACCTAAGTGGAACTC
(SEQ ID NO: 40)
HD-ptxBR ACTGCCTTCAGGAGAAAGTCAAAG
(SEQ ID NO: 41)
HD-msxF CCTTTTCTAGTGCATTTTGTGGC
(SEQ ID NO: 42)
HD-msxR GGTAATCGGTTTCCATAGCACATC
(SEQ ID NO: 43)
HD-Lmx1aAF CTGCTTATTTTGCTGTGGTTTG
(SEQ ID NO: 44)
HD-Lmx1aAR CCTTCTCTTGCTCTCATTTCTG
(SEQ ID NO: 45)
HD-Lmx1bAF CCAAACAAACGCACATCATCAG
(SEQ ID NO: 46)
HD-Lmx1bAR CACAAAAGCCAGGGAGTTCATTG
(SEQ ID NO: 47)
HD-Lmx1bBF GGCAGAGGAACAGAAAGAGGAAG
(SEQ ID NO: 48)
HD-Lmx1bBR ATGAAAGTGCTCGTGGTGTGGC
(SEQ ID NO: 49)
HD-Ngn2F TAGTGTATCCGCACAAAGGGGG
(SEQ ID NO: 50)
HD-Ngn2R AATCGCTGAACCAGGAGACAAAC

### Example 2

### Wnt1 Directly Regulates the Expression of Lmx1a and Otx2 During mDA Differentiation

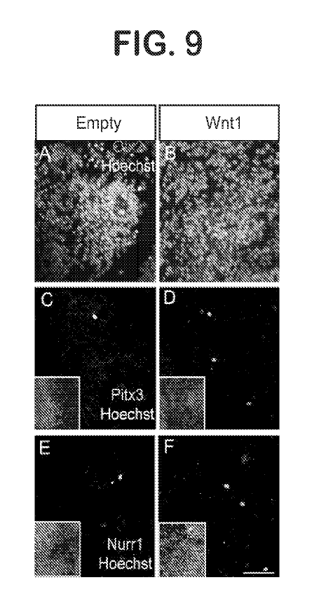

J1 ES cells were differentiated in vitro and infected with empty or Wnt1-expressing retrovirus at the NP stage (FIG. 8). To clearly see the effect of transgene expression without masking their effect by culture conditions, suboptimal condition were used without any DA-inducing factors. Cells were further differentiated and analyzed at day 3 of the neuronal differentiation (ND) stage (termed ND3 herein). This is the time point of active mDA neurogenesis and differentiation in this stem cell culture bioassay, thus optimal to analyze the expression of potential mDA regulators/targets. Quantitative real time PCR (qPCR) analysis revealed that forced Wnt1 expression significantly increased mRNA levels of Otx2, Pitx3 and to a greater extent Lmx1a (FIG. 1B), but not those of FoxA2, Nurr1 or Msx1. The lack of an effect on the expression of these genes at this early time point suggests that they are not the direct targets of Wnt1 signaling, though they may be regulated by genes further downstream. Consistent with this mRNA analysis, immunocytochemisty analysis revealed an increased number of Lmx1a⁺ cells after Wnt1 overexpression (FIG. 1C-D) from 7.05±0.78 to 16.20±1.11 (% Lmx1a⁺ cells/Hoechst⁺ cells; P<0.05). In addition, Otx2⁺ or Pitx3⁺ cell numbers, but not Nurr1⁺ cell numbers, were significantly increased after Wnt1 overexpression (FIG. 9).

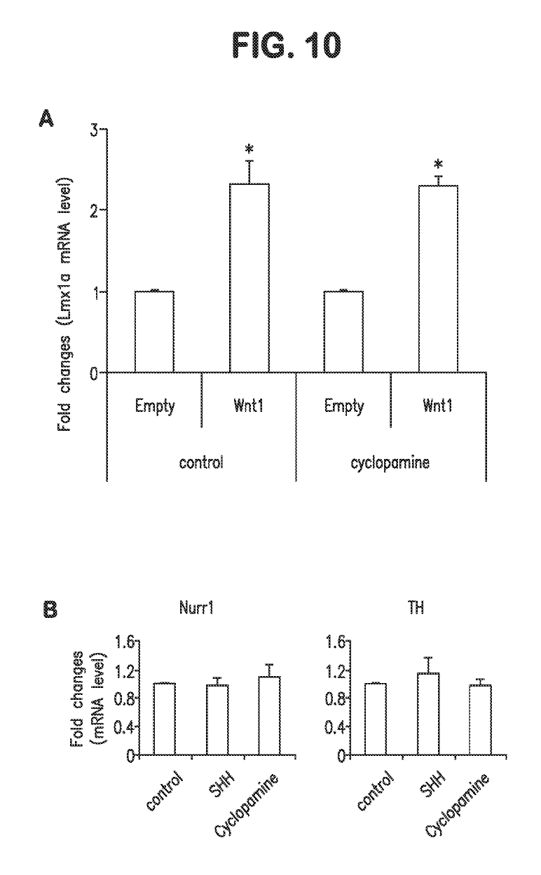

It was previously shown that SHH treatment could ventralize chick intermediate midbrain explants, accompanied by induction of Lmx1a and other ventral midbrain phenotype (Andersson et al., 2006b). Thus, it was tested whether Wnt1 can still induce Lmx1a in the presence of the SHH signaling inhibitor cyclopamine (Kittappa et al., 2007) and it was found that Wnt1 induced Lmx1a independent of SHH signaling (FIG. 10A). It was next tested whether acute treatment with SHH or cyclopamine had an immediate effect on Lmx1a expression. ES-derived NP cells were treated with 500 ng/ml SHH or 1 μM cyclopamine for 6 hours and analyzed by qPCR. While these treatments led to corresponding changes in Gli1 mRNA levels, there was no significant changes in Lmx1a mRNA levels (FIG. 1E) as well as TH or Nurr1 mRNA levels (FIG. 10B), suggesting that these genes are not direct targets of the SHH signaling.

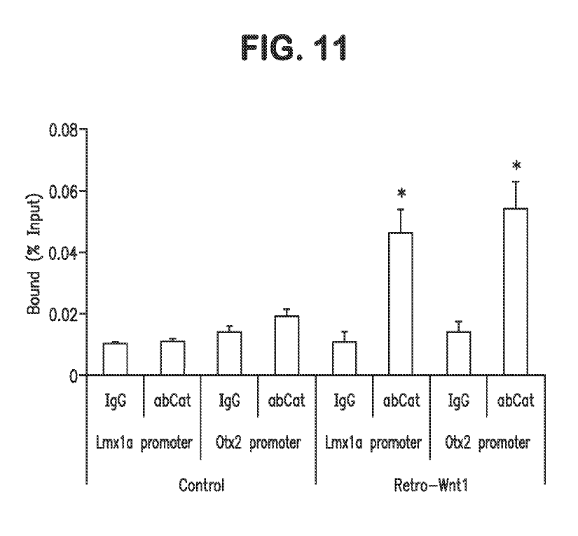

To address whether Wnt1 directly regulates any of these potential targets via the canonical Wnt signaling pathway, chromatin immunoprecipitation (ChIP)-qPCR analysis was performed. At day 1 of the NP stage, in vitro differentiated ES cells were transduced with retroviral Wnt1, treated with 15 mM LiCl at day 3 to stabilize β-catenin, and then fixed for ChIP at day 4. ChIP was performed either with a control antibody or with anti-β-catenin antibody. qPCR analysis showed significant binding of β-catenin complex to the well conserved TCF/LEF binding site in the Lmx1a promoter, but not to another potential TCF/LEF site in the third intron of Lmx1a, showing the specificity of β-catenin binding in the assay system (FIG. 1F). For the Otx2 promoter, ChIP-qPCR analysis showed that there is direct association of the β-catenin complex to its well conserved TCF/LEF sites during mDA differentiation (FIG. 1F). The c-myc promoter's TCF/LEF binding sites was used as positive control (Yochum et al., 2007), and comparable binding with the Lmx1a promoter and the Otx2 promoter was observed (FIG. 1F). In the absence of LiCl treatment, the ChIP experiment yielded comparable results (FIG. 11), suggesting that Wnt1 expression alone is sufficient for stabilizing the β-catenin complex in this system. Furthermore, this binding of β-catenin complex was Wnt1-dependent (FIG. 11). For the Pitx3 promoter, the regulation by Wnt1 appears to be indirect, since well-conserved TCF/LEF binding sites on the Pitx3 promoter could not be found, even though there is a possibility of regulation by a long-range enhancer.

To further test whether these direct downstream targets are bound by the β-catenin complex in vivo during embryonic development, the ChIP analysis was performed using dissected VM of E11.5 embryo. This analysis confirmed that the Lmx1a and Otx2 promoters are physically associated with the β-catenin complex (FIG. 1G), supporting the in vitro data that Lmx1a and Otx2 are direct targets of the Wnt1 signaling pathway during mDA development.

### Example 3

### Lmx1a Directly Regulates Wnt1 Expression During mDA Differentiation

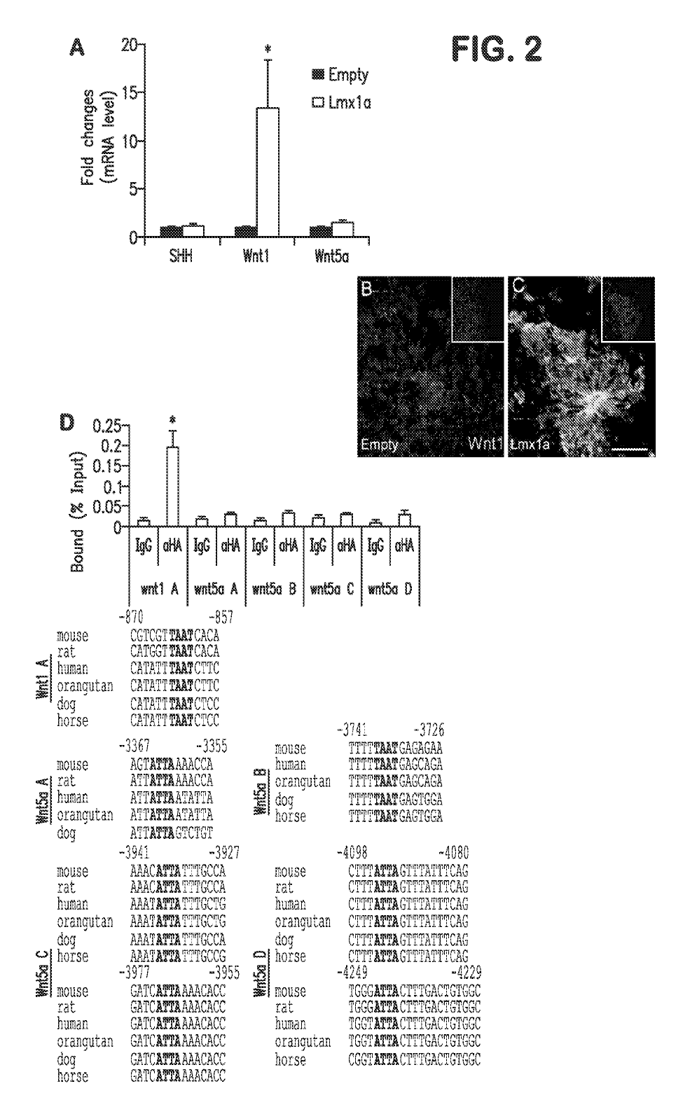

Since Lmx1a showed the most robust effect by Wnt1 overexpression, experiments were carried out to identify the downstream targets of Lmx1a. J1 ES cells were differentiated in vitro, infected with empty or Lmx1a-expressing retrovirus at the NP stage, and analyzed at ND3 after further differentiation. QPCR analysis showed that Lmx1a dramatically increased expression of Wnt1, but not that of SHH or Wnt5a (FIG. 2A). Lmx1a was also overexpressed using episomal vector and similar results were observed (data not shown). In addition, immunocytochemical analysis showed that exogenous Lmx1a expression robustly increased the numbers of Wnt1⁺ cells (FIG. 2B-C).

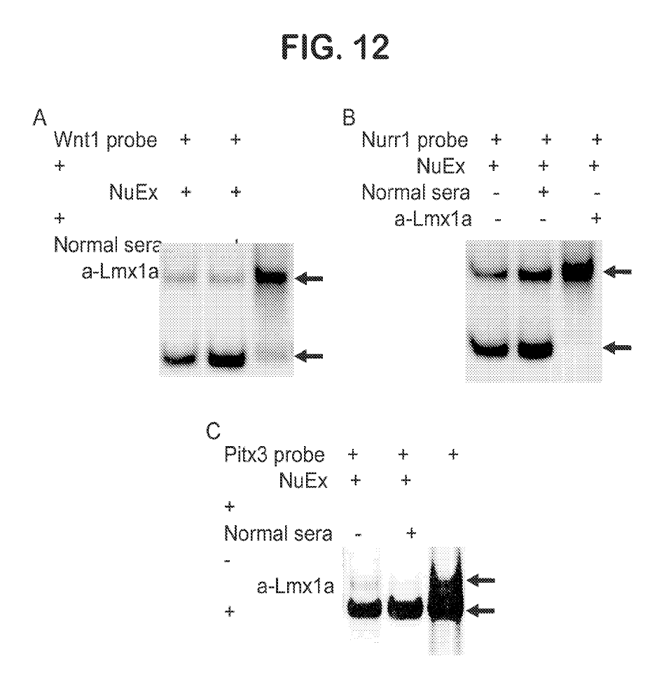

The possibility that Lmx1a directly regulates the expression of Wnt1 by ChIP-qPCR analysis was then tested. In vitro differentiated J1 cells at the NP stage were transduced with retrovirus expressing HA-tagged Lmx1a, and harvested for ChIP at ND3. Crosslinked chromatin complex was immunoprecipitated using anti-HA antibody or control IgG, and analyzed by qPCR. There was significant Lmx1a binding to the well-conserved homeodomain binding site in the Wnt1 promoter, but not to 6 well-conserved sites contained in 4 PCR fragments on the Wnt5a promoter, demonstrating the specificity of in vivo Lmx1a binding (FIG. 2D). Importantly, this ChIP data is consistent with the overexpression data that Lmx1a regulates Wnt1, but not Wnt5a (FIG. 2A-C), further supporting the validity of the ChIP analysis. The binding of Lmx1a to the Wnt1 promoter by an independent method was confirmed (electrophoretic mobility shift assay (EMSA)), and specific DNA-protein complex formation which was supershifted by anti-Lmx1a antibody was observed (FIG. 12A). Taken together, the results reveal the presence of a tight autoregulatory loop between Wnt1 and Lmx1a during mDA differentiation of ES cells.

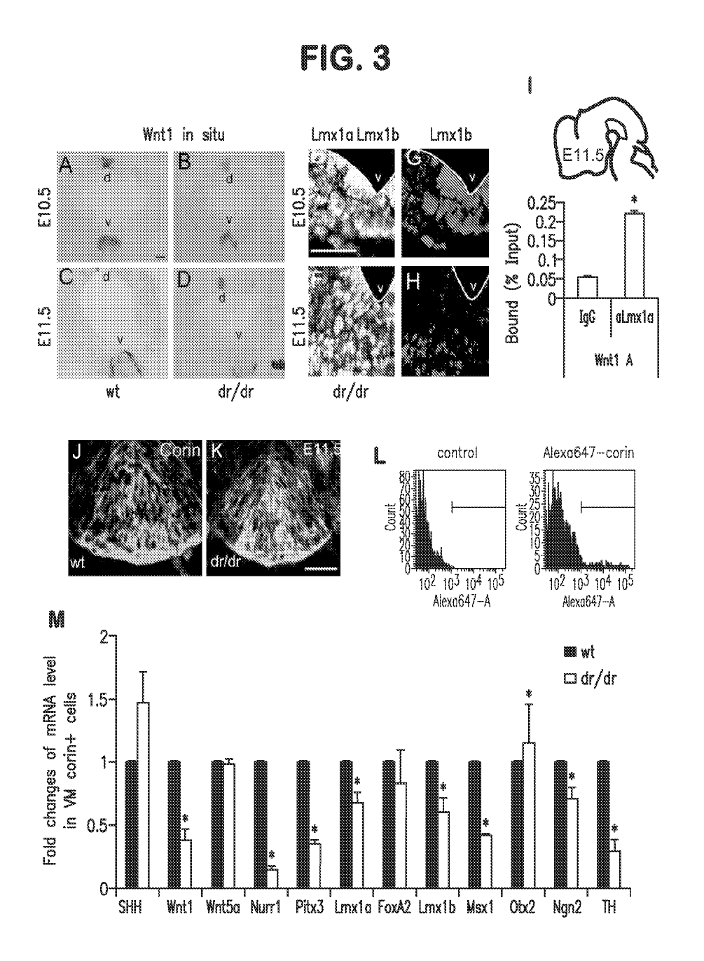

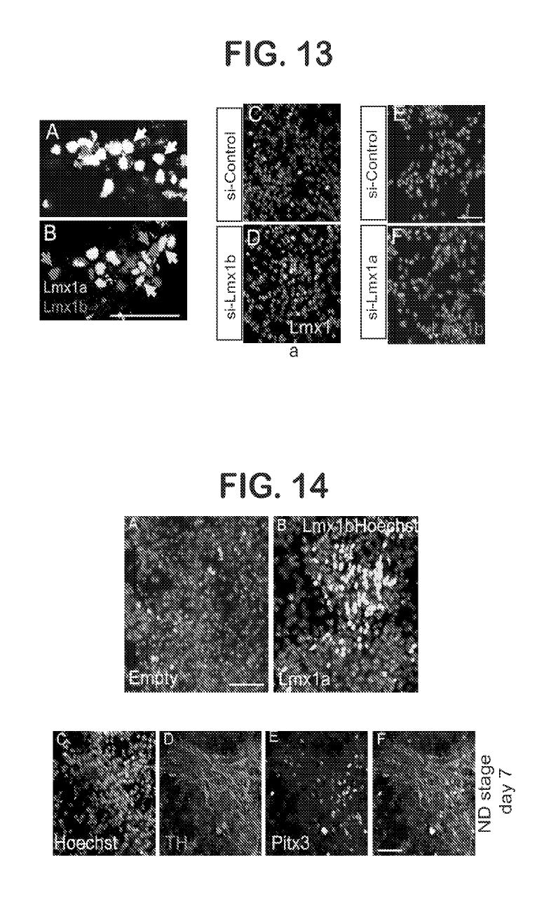

Next, it was tested whether Lmx1a regulates the expression of Wnt1 during mouse embryonic midbrain development in vivo, using the wildtype (wt) and dreher (dr/dr) mice (Millonig et al., 2000). In situ hybridization analysis of littermate wt vs. dr/dr embryos showed that Wnt1 expression is compromised by Lmx1a mutation in developing midbrain (FIG. 3A-D). At E11.5, this defect was more evident, although Wnt1 expression was partially spared in the ventral most part (FIG. 3C-D). One possible explanation of this residual Wnt1 expression is the functional compensation by Lmx1b, which is expressed in the entire mDA domain at E10.5 (FIGS. 3E and 3G) and in the ventral most part at E11.5 (FIGS. 3F and 3H), which will be further discussed later. The specificity of the antibodies against Lmx1a and Lmx1b is shown in FIG. 13. To further test whether there is a direct interaction between Lmx1a and the Wnt1 promoter during embryonic development, ChIP analysis was performed using dissected VM of E11.5 embryo and it was found that the Wnt1 promoter is physically associated with Lmx1a in developing VM (FIG. 3I), confirming the presence of the Wnt1-Lmx1a autoregulatory loop in the embryo as well as during ES cell differentiation.

To quantitatively analyze the effect of Lmx1a mutation on gene expression in vivo, E11.5 mesencephalic floor plate (mFP) cells were purified, which generate mDA neurons (Kittappa et al., 2007; Ono et al., 2007), from littermates wt and dr/dr embryos. To purify mFP cells, fluorescent activated cell sorting (FACS; FIG. 3L) was performed using antibody against corin, a cell surface marker specifically expressed in developing FP cells (FIG. 3J-K) (Ono et al., 2007). mRNA analysis showed that Lmx1a mutation caused a significant decrease (approximately 60%) in expression of Wnt1, but not that of Wnt5a (FIG. 3M), consistent with the result from ES cell differentiation (FIG. 2A). Mild decrease in Lmx1a, Lmx1b and Ngn2 mRNA levels in the dr/dr embryos was observed, consistent with the previous study (Ono et al., 2007).

### Example 4

### Lmx1a Directly Binds the Promoter Element(s) and Regulates Expression of Nurr1 and Pitx3

In addition to Wnt1 gene regulation by Lmx1a mutation, there was significant reduction in the expression of Nurr1 and Pitx3 (FIG. 3M). In the dr/dr embryo, this downregulation of Nurr1 and Pitx3 could be an indirect effect of defective DA neuron differentiation. Alternatively, it may be caused by direct regulation of Lmx1a. To address these possibilities, it was tested whether Lmx1a directly regulates the expression of Pitx3 and Nurr1 during ES cell in vitro differentiation. Retroviral Lmx1a expression increased the expression of Pitx3, Nurr1 and Lmx1b, whereas it failed to significantly affect the expression of Otx2, FoxA2 or En1 at ND3 (FIG. 4A). Significant increase in Msx1 and Ngn2 mRNA levels was also observed, consistent with a previous study (Andersson et al., 2006b). This experiment was repeated using an episomal Lmx1a expression system and obtained similar results (data not shown). Immunocytochemical analysis showed that exogenous Lmx1a expression increased the number of Nurr1⁺ (FIG. 4B-C; from 2.07±0.38 to 3.88±0.46% Nurr1+ cells/Hoechst+ cells) and Pitx3⁺ cells (FIG. 4E-F; from 0.58±0.40 to 3.50±0.33% Pitx3⁺ cells/Hoechst⁺ cells) as well as Lmx1b⁺ cells (FIG. 14A-B). Interestingly, many Pitx3⁺ cells and Nurr1⁺ cells were not yet positive for TH at ND3 (FIGS. 4C and 4F), suggesting that the increase in Pitx3 and Nurr1 gene expression is a direct effect, but not the byproduct of increased mDA neurons. At a later time point (ND7), the majority of Pitx3⁺ cells became TH⁺, suggesting that Lmx1a induced Pitx3 expression in immature DA neurons (FIG. 14C-F). In addition, Lmx1a expression significantly increased the % TH⁺ cells/β-tubulin⁺ cells from 0.87±0.21 to 2.98±0.84 without supplementing the culture with SHH, unlike the previous report which the effect of Lmx1a on DA induction was strictly dependent upon addition of SHH to the culture (Andersson et al., 2006b). Endogenous SHH expression at the NP stage may explain such difference.

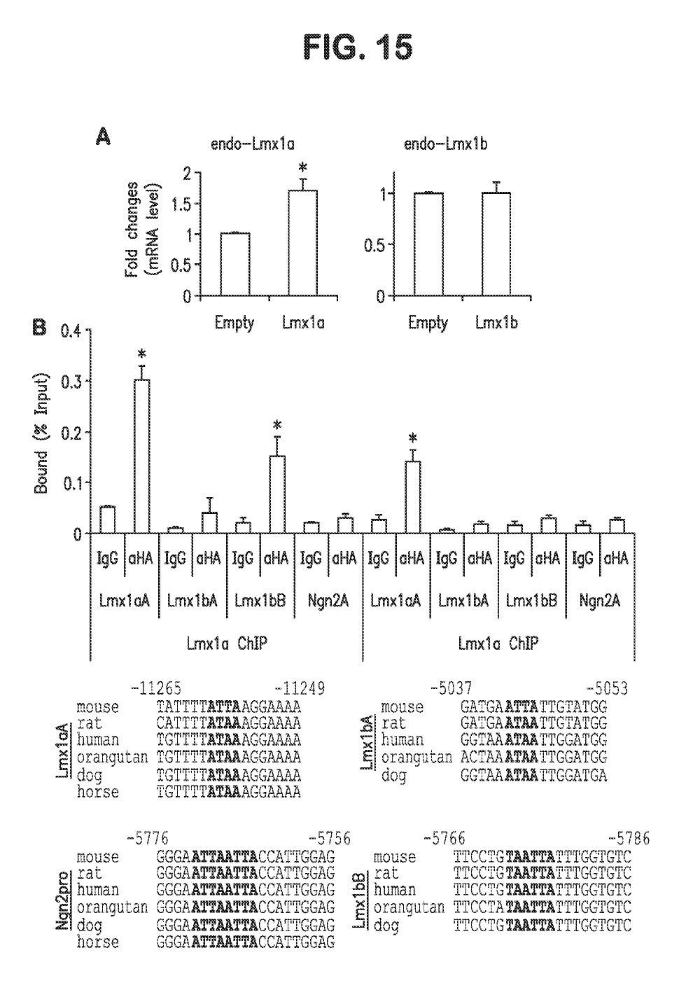

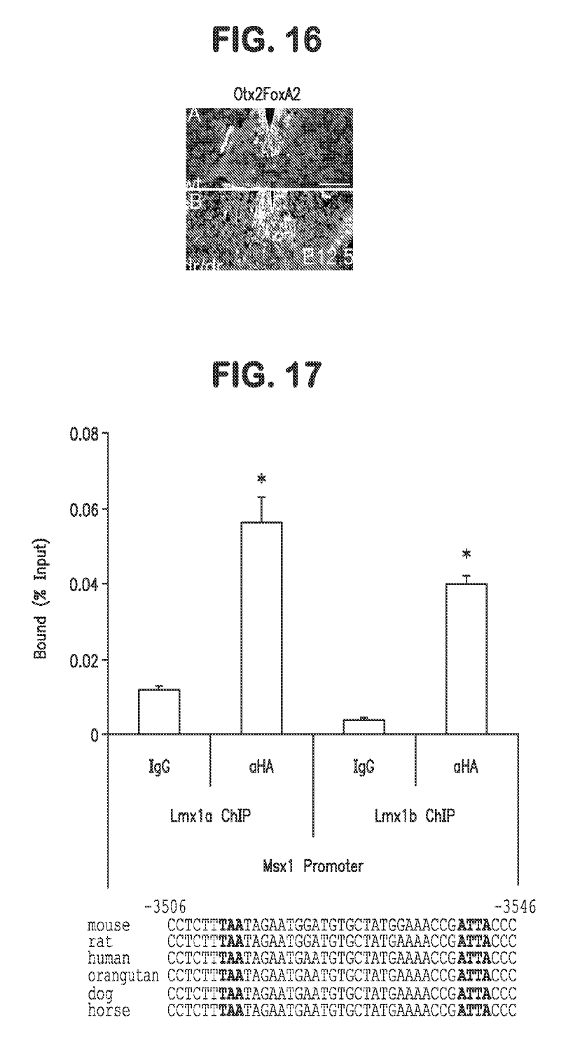

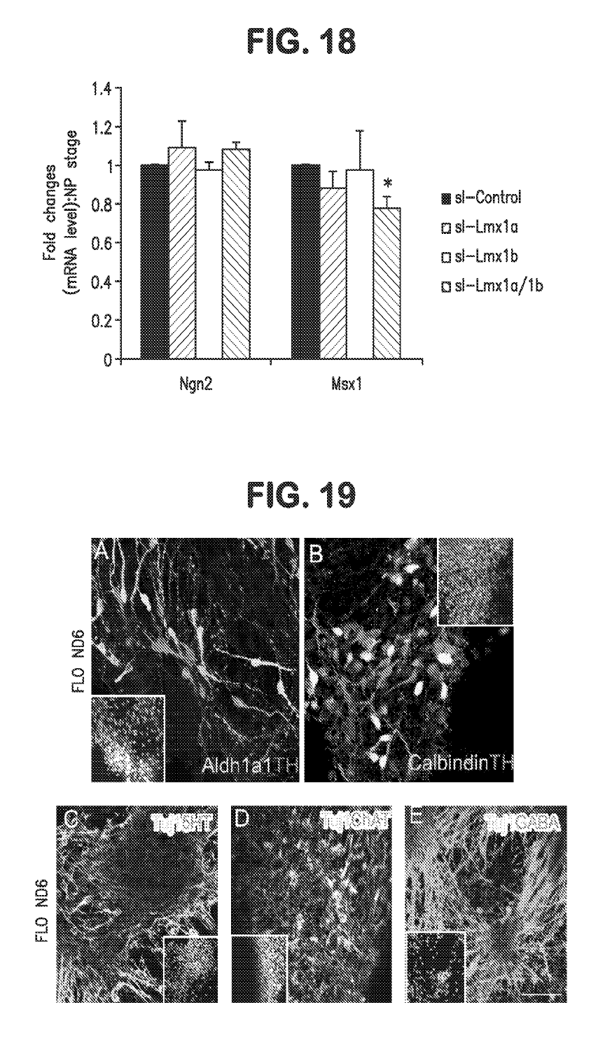

To further address whether Lmx1a directly regulates gene expression of the mDA regulators, Nurr1 and Pitx3, ChIP analysis was performed. It was found that Lmx1a significantly bound to Nurr1A and Nurr1C PCR fragments, but not the Nurr1B fragment (FIG. 4D), and the specific binding of Lmx1a by supershift EMSA was confirmed (FIG. 12B). For the Pitx3 promoter, significant Lmx1a binding to Pitx3A was observed, but not Pitx3B PCR fragment (FIG. 4G), and also confirmed it by supershift EMSA (FIG. 12C). ChIP was also performed to test whether Ngn2 is directly regulated by Lmx1a, but observed no significant binding (FIG. 15B).

To further confirm the regulation of Nurr1 and Pitx3 by Lmx1a during embryonic midbrain development, immunohistochemistry and stereological analysis were performed on littermate wt and dr/dr embryos. The number of Nurr1⁺ and Pitx3⁺ cells were counted in the entire mDA domain in every 6^{th} coronal VM section, using the Stereolnvestigator image capture equipment and software. Significant decreases in Nurr1⁺ and Pitx3⁺ cell numbers in dr/dr embryos were found compared to littermate wt embryos (FIG. 4H-M), whereas there was no significant difference in the FoxA2⁺ or Otx2⁺ cell numbers between wt and dr/dr embryos. Taken together, the results strongly suggest that Lmx1a directly regulates Nurr1 and Pitx3, but not FoxA2 or Otx2 both in mDA differentiation of ES cells and in embryonic midbrain development.

### Example 5

### Lmx1a and Lmx1b have Overlapping Functions in Regulating mDA Regulators

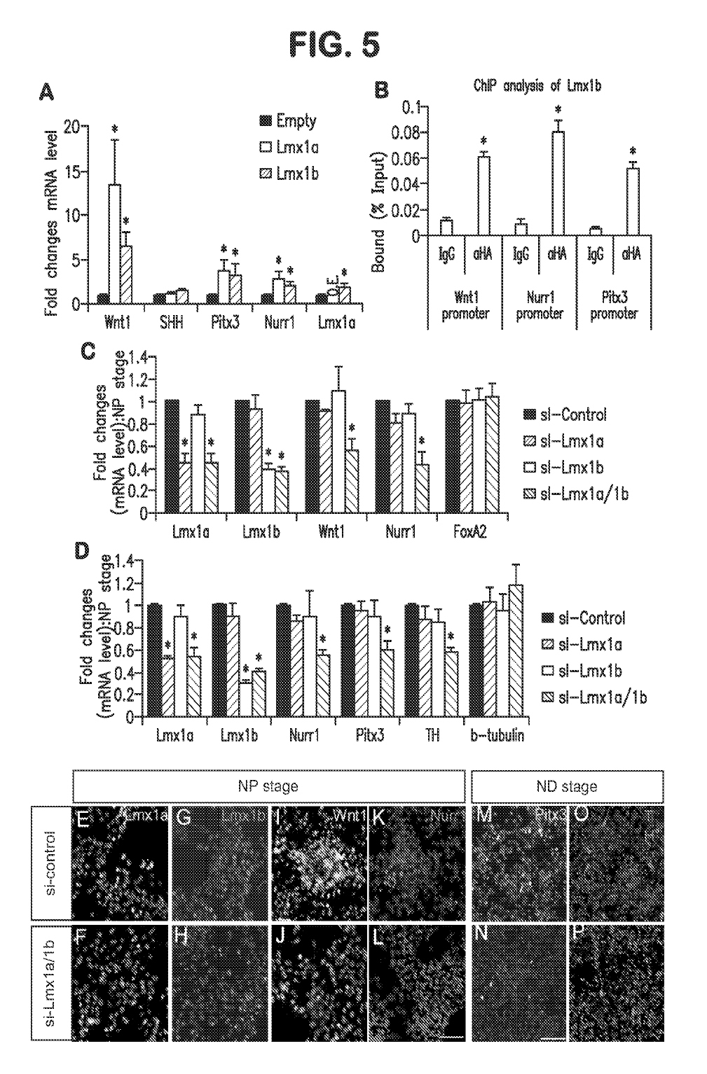

Compared to the robust induction of mDA differentiation in ES cells by Lmx1a, dreher mice displayed only mild dysregulation of mDA development. This could be explained either by lack of functional significance of Lmx1a during embryonic mDA development or by the presence of another gene with redundant function. For the latter possibility, Lmx1b is one such candidate, because (1) it is expressed in the same domain as Lmx1a during mDA development and (2) it is highly related to Lmx1a with 61% overall amino acid identity (Hobert and Westphal, 2000). Thus, to explore whether Lmx1b and Lmx1a share some redundant functions in mDA differentiation, the effect of Lmx1a and Lmx1b overexpression during in vitro differentiation of ES cells was compared. J1 ES cells were differentiated in vitro, infected with Lmx1a- or Lmx1b-expressing retrovirus at the NP stage, and analyzed at ND3. In line with Wnt1's residual expression pattern in dr/dr embryos (FIG. 3A-H), both Lmx1a and Lmx1b upregulated Wnt1 expression (FIG. 5A). SHH expression was unaffected by either gene, while both Pitx3 and Nurr1 expression were upregulated by Lmx1a or Lmx1b (FIG. 5A), showing the redundant function of Lmx1a and Lmx1b in target gene regulation. Interestingly, Lmx1b expression mildly but significantly upregulated Lmx1a expression (FIG. 5A). ChIP analysis showed that Lmx1b binds to the Lmx1a promoter and also Lmx1a binds to the Lmx1b promoter, indicating cross-regulation between these two genes (FIG. 15B). In addition, it was also examined whether there is any self-regulation of Lmx1a or Lmx1b. qPCR analysis using endogenous message-specific primers revealed that Lmx1a regulates itself, but Lmx1b does not (FIG. 15A). Consistent with this, it was observed that Lmx1a but not Lmx1b specifically binds to the well conserved binding site within its own promoter (FIG. 15B).

Observed cross-regulation between Lmx1a and Lmx1b raised the possibility that Lmx1b regulates target genes indirectly through Lmx1a. Thus, to test whether Lmx1b can directly regulate target genes, ChIP-qPCR analysis was done following transduction with retrovirus expressing HA-tagged Lmx1b. It was found that Lmx1b significantly bound to the promoters of Wnt1, Nurr1 and Pitx3 (FIG. 5B), though milder than Lmx1a. The binding of Lmx1a or Lmx1b to the Msx1 promoter was also tested, and it was found that they both bind to the well conserved homeodomain binding sites residing at −3.5 kb upstream of the Msx1 gene (FIG. 17).

To further study the redundant function between Lmx1a and Lmx1b, experiments were carried out to knock down these genes using gene-specific siRNA approach. ES cell-derived NP cells were treated with SHH and FGF8 for 4 days to induce/proliferate mDA NPs, and then transfected with control siRNA, Lmx1a siRNA, Lmx1b siRNA or both Lmx1a/1b siRNAs using Nucleofector (Amaxa) and analyzed after 30 hours. Transfection of each siRNA treatment significantly reduced the mRNA level of Lmx1a or Lmx1b (FIGS. 5C and E-H). Transfection of single siRNA did not have significant effect on Wnt1 or Nurr1 gene expression. This insignificant effect is in contrast with the robust induction effect observed in overexpression experiment (FIG. 5A). This can be explained by incomplete knockdown by siRNA and/or nonphysiological overexpression effect caused by retroviral transduction. However, when both genes were knocked down, there was significant decrease in the target gene expression (FIGS. 5C, 5I-L and 18), demonstrating that Lmx1a and Lmx1b compensate each other's function in regulating mDA regulator genes. Since Pitx3⁺ cells were not yet detectable at this NP stage, the gene knockdown experiment was repeated at ND stage cells. ES cell-derived cells were treated with SHH and FGF8 for 4 days, differentiated in N3 media for 2 days, transfected with siRNA and analyzed by qPCR analysis 30 hrs after transfection. siRNA treatment to each genes significantly reduced the mRNA level of Lmx1a or Lmx1b (FIG. 5D). Only when both Lmx1a and Lmx1b genes were knocked down, there was significant decrease in Nurr1 and Pitx3 gene expression (FIGS. 5D and M-N). Furthermore, knock down of both genes also downregulated TH mRNA level and TH⁺ cell numbers (FIGS. 5D and O-P).

### Example 6

### Wnt1-Lmx1 Autoregulatory Loop Induces mDA Differentiation Synergistically with the SHH Signaling Pathway

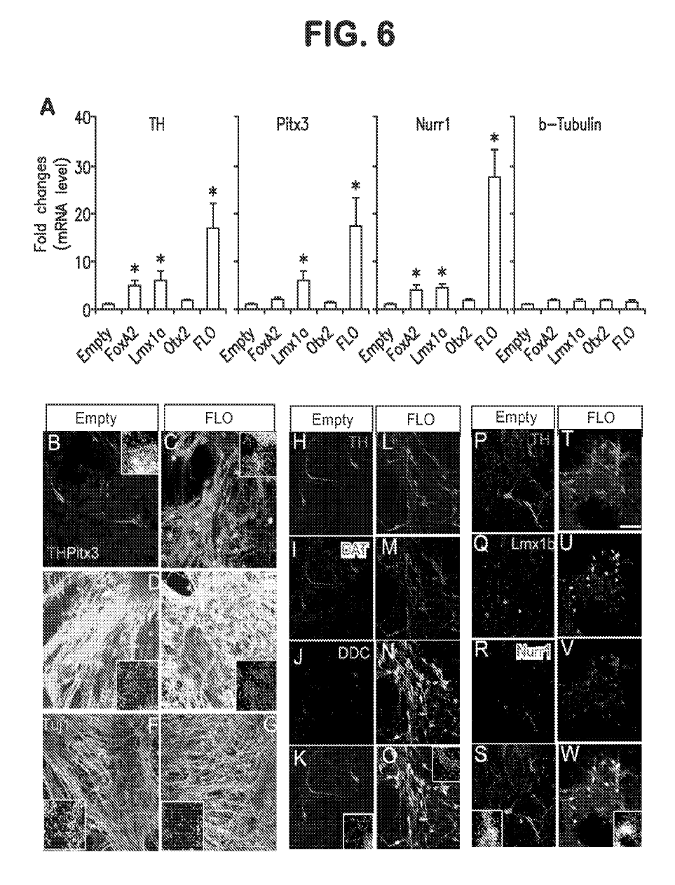

A salient finding of this study is the tight autoregulatory loop between Wnt1 and Lmx1a during mDA differentiation of ES cells as well as during embryonic midbrain development. This autoregulatory loop, in turn, directly regulates Otx2 expression, through the canonical Wnt signaling pathway, and Nurr1 and Pitx3 expression, through Lmx1a. This finding suggests that activation of both Wnt and SHH signaling pathways by exogenous expression of direct downstream targets of these pathways (i.e., Otx2, Lmx1a and FoxA2) may synergistically induce mDA differentiation. To prove it, ES-derived NPs were transduced with FoxA2-, Lmx1a- or Otx2-expressing retroviruses, either alone or together. Indeed, when all three key mediators (Lmx1a, Otx2 and FoxA2) were overexpressed, a robust synergistic induction of the mDA marker genes, TH, Pitx3 and Nurr1 was observed (FIG. 6A), as examined by qPCR analysis. Immunocytochemical analysis also showed significant increase in mDA neurons as shown by increase in the number of cells expressing both TH and Pitx3 (FIG. 6B-E). However, there was no significant change in β-tubulin⁺ neuronal cell numbers or GFAP⁺ astrocyte cell numbers (FIG. 6F-G). Further analysis showed that TH⁺ neurons generated by activation of both signaling pathways represent mature DA neuronal phenotype assessed by coexpression of DAT and DDC, but not by empty vector-transduction (FIG. 6H-O). In the three factor-transduced cells, the majority of TH⁺ neurons also coexpressed Lmx1b and Nurr1, confirming their mDA phenotype, but not in the empty-vector-transduced cells (FIG. 6P-W). Three factor-transduced cells contained both A9-like (Aldh1al⁺) and A10-like (Calbindin) mDA neurons (FIG. 19A-B). In addition, other non-DA neurons such as serotonergic (5HT⁺), cholinergic (ChAT⁺) or GABAergic (GABA⁺) neurons were similarly observed after in vitro differentiation of both empty vector-transduced and three factor-transduced cells (FIG. 19C-E; data not shown). Cell counting analysis showed that there was a significant increase in % TH⁺ cells/β-tubulin⁺ cells by three-factor transduction from 4.85+024 to 26.30+0.49 (from 2.35+0.11 to 13.22+0.57% TH⁺/Hoechst⁺ cells).

### Example 7

### Guided Differentiation of Stem Cells by Direct Protein and/or mRNA Delivery Toward Specific Cell Lineages

It can be shown that stem cells can be safely and efficiently manipulated for guided differentiation towards specific cell lineage(s) by delivering key TF proteins attached to cell penetrating peptides (CPPs) or protein transduction domains (PTD) in a combinatorial and temporally-regulated manner. This new platform, which can be termed “gene-less engineering”, can provide unlimited and clinically viable cell source for study and treatment of human diseases such as PD.

7.1 Establishment of Stable HEK 293 Cell Lines Expressing Recombinant Nurr1, Pitx3, or Lmx1a Protein Fused to a CPP for Direct Protein Delivery

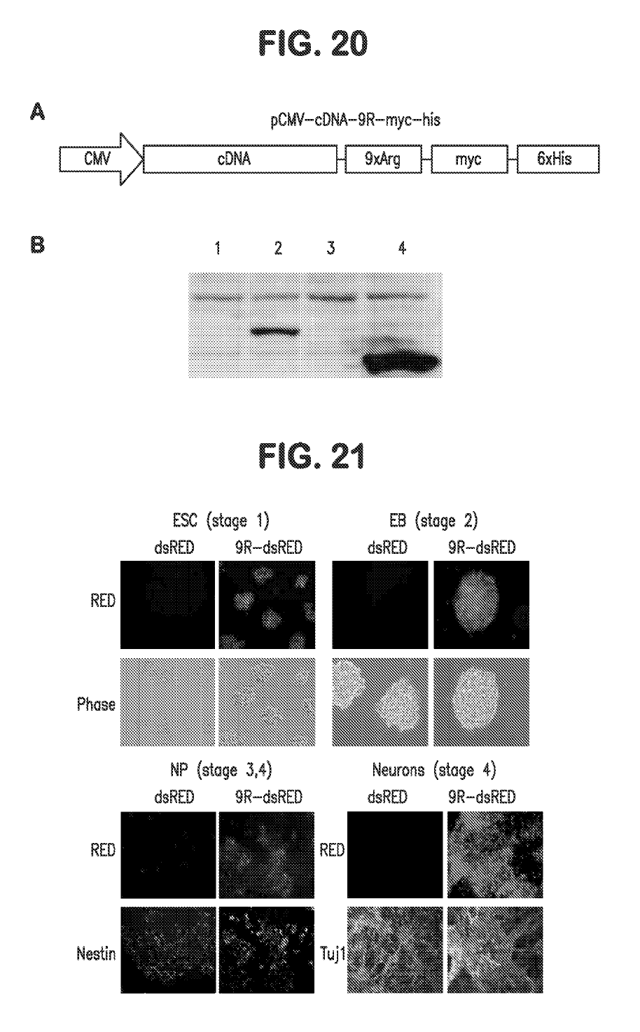

In addition to the E. coli. expression system, mammalian expression systems can be used. Stable HEK293 cell lines expressing high levels of each of the TFs (Nurr1, Pitx3, and Lmx1a) fused with a CPP (a 9 arginine repeat; 9R), the myc tag, and a 6 histidine repeat (6H) at the C-terminus (pCMV-cDNA-9R-myc-6H; FIG. 20) can be prepared. All three expression vectors have been generated and confirmed that they are in frame by sequencing analyses. In the case of Nurr1, both wild type and the recently identified degradation-resistant mutant form consisting of a serine 347 to alanine substitution were generated. HEK293 cells can be transfected with each of these vectors and stable lines from neomycin-resistant colonies can be isolated, expressing high levels of the recombinant proteins as determined by western blot analysis using myc antibodies. Recently the applicants used this expression system to establish stable HEK293 cell lines expressing high levels of all four reprogramming proteins (Oct4, Sox2, Klf4, and c-Myc), strongly supporting the feasibility of our approach (Kim et al., 2009).

7.2 Purification of Key Recombinant TF Proteins

The cultures of stable HEK293 cell lines that robustly express each of the recombinant proteins can be expanded. Each protein can be purified by nickel affinity chromatography (for this purpose, each recombinant protein contains a 6 histidine repeat at the C-terminus; FIG. 20). Stable HEK293 cell lines expressing Nurr1, Pitx3, and Lmx1a have been generated and confirmed to have robust expression of Nurr1 and Pitx3 (FIG. 20B). Further, stable clones expressing Lmx1a and mutant form of Nurr1 can be isolated. These clones can be cultured in large quantity and harvested cells can be suspended in lysis buffer and sonicated on ice. The supernatant fraction can be loaded onto a Ni-NTA column (Qiagen) and washed with buffer solution. The bound proteins can be eluted with elution buffers consisting of lysis buffer containing increasing amounts of imidazole (50-250 mM). Positive fractions can be confirmed by immunoblotting assay, pooled, and dialyzed at 4° C. using 1×PBS. Using this approach, all four recombinant reprogramming proteins have been purified and successfully generated additional mouse iPSC lines.

7.3 Stability of Recombinant TF Proteins

Each recombinant protein needs to be stable enough inside the cells to exert its functional effect on DA neuron differentiation. A protein's stability can be checked with myc antibodies by both western blot and immunocytochemistry analyses. A vector expressing the degradation-resistant mutant of Nurr1 has been prepared. Mutants that are resistant to protein degradation pathways can also be prepared by identifying putative ubiquitin acceptor site(s) based on protein sequence analysis and ubiquitination assays.

7.4 Guided Differentiation of mESCs by Direct Delivery of Key TF Protein

The optimal time for TF protein delivery into mESC-derived cells during in vitro differentiation for optimal DA neuron generation can be determined. A preliminary study to test if red fluorescent protein fused with 9R (9R-dsRED) can penetrate ESC-derived cells at the ESC, EB, NP, and differentiated cell stages has been performed. It was found that 9R-dsRED was efficiently delivered into ESC-derived cells at all stages with almost 100% efficiency within a few hours while dsRED by itself did not enter the cells (FIG. 21). Together with previous results showing that reprogramming proteins fused with 9R can efficiently penetrate cells, these findings show that these recombinant TF proteins can be efficiently delivered to any stage of ESC-derived cells.

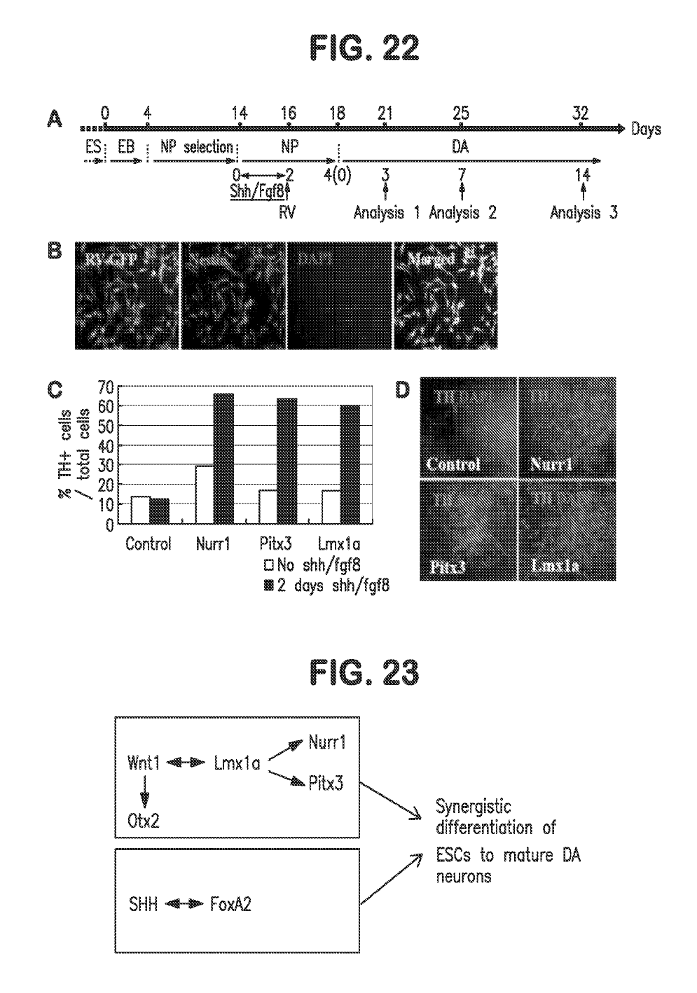

The J1 mESC line can be used for this purpose. Different amount of each of (partially) purified recombinant protein can be added to ESC-derived cells at different stages (e.g., at day 0, 2, 12, 16, and 18 of FIG. 22A). Following in vitro differentiation for 3, 7, or 14 days (Analysis 1-3 in FIG. 22A), the effect of each condition on the generation of DA neurons can be tested using immunocytochemistry and PCR analyses of specific marker genes such as tyrosine hydroxylase (TH) and dopamine transporter (DAT). These TF may work optimally at the NP stage. Different NP stages such as day 10, 12, 14, and 16 can also be tested for the treatment. The proteins can be delivered to the cells more than once. Differentiated ES cells can be treated with medium supplemented with 50 mM KCl and 0.1 mM Pargyline and the media can be collected after 30 minutes. DA contents can then be measured by reverse-phase HPLC. Optimal conditions of protein treatment for DA neuron differentiation can be then determined. The differentiated DA neurons for long-term periods (e.g., 1 to 6 months) can be cultured and tested for their survival and the expression of DA markers to determine if these DA neurons can be stably maintained.

7.5 Optimal Differentiation of mESCs into Mature DA Neurons by Combined Treatment of key TF Proteins:

Effect of a Combined Treatment of Nurr1 and Pitx3 Proteins.

Both of the Nurr1 and Pitx3 proteins can be introduced into mESCs and their effects on DA neuron differentiation can be analyzed. The treatment can start at day 14, 16, or 18 (FIG. 22) for different periods (e.g., 4 to 14 days) and the DA phenotype at day 25 and 32 (that is, 7 and 14 days of neuronal differentiation, respectively) can be examined. It is expected that Nurr1 and Pitx3 proteins will enhance DA neuron differentiation of mESCs. It can also be determined whether Nurr1 and Pitx3 protein treatments can induce the endogenous regulatory program to maintain the DA neuron phenotypes and survival.

Effect of a Combined Treatment of Lmx1a, Otx2, and FoxA2 Proteins.

As demonstrated in other Examples, Wnt1 and Lmx1a form an autoregulatory loop and directly control Otx2, Nurr1, and Pitx3 and that this Wnt1-Lmx1a pathway works co-operatively with the SHH-FoxA2 pathway during DA neuron development (FIG. 23). The combined expression of the immediate target TFs (Lmx1a, Otx2, and FoxA2) synergistically induced DA neurons from mESCs. These three proteins can be added to the mESCs together. The treatment, for example, can be started at day 10, 14, or 16 (FIG. 22) and the DA phenotype can be examined at day 25 and 32. Since these factors are for auto- and cross-regulatory loops and cascades, they will induce the intrinsic, self-sustainable program for DA phenotype induction and maintenance.

Effect of a Combined Treatment of Lmx1a, Otx2, FoxA2, Nurr1, and Pitx3 Proteins in a Temporally Regulated Manner.

The effect of treating cells with all five TF proteins in a temporally regulated manner can further be tested. The cells can be treated with early factors (Lmx1a, Otx2, and FoxA2) at an early NP stage and with late factors (Nurr1 and Pitx3) at a later NP stage. The effect of this five-factor treatment on the induction of GFP⁺ neurons from Pitx3-GFP knock-in mESCs can be compared to the above three- and two-factor treatments. To determine whether these GFP neurons have acquired a midbrain DA neuronal fate, immunocytochemistry of in vitro differentiated ESCs can be performed using antibodies against DA markers such as DDC, DAT, and VMAT2. In addition, these cells can be stained with antibodies against A9-specific markers such as ADH2 and Girk2. Furthermore, expression of other cell type markers, e.g., DBH and NET (noradrenergic subtype), TPH and 5-HT (serotonergic subtype), GFAP (glial cell type) can be examined. In addition, mRNAs from in vitro differentiated ES cells can be isolated at different stages and the expression of each marker gene can be examined by semi-quantitative and real-time PCR analyses. Using the optimal conditions, efficient DA neuron differentiation of the J1 mESC line can be confirmed.

7.6 Protein Engineering of iPSCs using Optimal Conditions of Key TF Protein Treatment for In Vitro Differentiation to Mature DA Neurons

Using the direct protein delivery method (Kim et al., 2009), the TFs can be delivered to six protein-induced iPSC (p-iPSC) previously prepared by the applicants. The p-iPSC lines can then be examined for their in vitro differentiation into DA neurons using the 5-stage method (FIG. 22).

These protein-engineered cells can improve behavioral and functional defects which can be tested in a rodent model of PD, following intrastriatal transplantation of DA neurons from protein-engineered ESC and/or iPSCs in aphakia mice. The aphakia mouse is a valid and convenient genetic PD model (Huang et al., 2005 and Ardayfio et al, 2008). Since aphakia mice can breed as homozygote pairs, a large number of animals are readily available for systematic behavioral analyses with minimal individual fluctuations. Furthermore, it can provide an ideal platform to test whether the same species ESCs/iPSCs-derived DA neurons can function in the same species animal model without the need for immune suppression. ESC/iPSC-derived cells can be transplanted at the early-differentiated stage (e.g., day 21 of FIG. 1A) into the striatum of aphakia mice, using a 22-gauge, 2.5 μl Hamilton syringe and a Kopf stereotaxic frame. Transplanted aphakia mice can be analyzed for graft volumes, cell survival, teratoma formation, their phenotypic expression, morphological and differentiation properties. Locomotor activity can be measured by a gross motor function test. Then, more nigrostriatal pathway-sensitive motor behavioral tests such as cylinder, challenging beam, and pole tests can be performed at 1, 2, and 6 months post transplantation. Animals exhibiting robust functional improvements following transplantation can be further analyzed for their graft volume, phenotypic expression of mDA markers, host integration, and mature neuronal morphology.

### REFERENCES

Andersson, E., Jensen, J. B., Parmar, M., Guillemot, F., and Bjorklund, A. (2006a). Development of the mesencephalic dopaminergic neuron system is compromised in the absence of neurogenin 2. Development 133, 507-516.
Andersson, E., Tryggvason, U., Deng, Q., Friling, S., Alekseenko, Z., Robert, B., Perlmann, T., and Ericson, J. (2006b). Identification of intrinsic determinants of midbrain dopamine neurons. Cell 124, 393-405.
Ang, S. L. (2006). Transcriptional control of midbrain dopaminergic neuron development. Development 133, 3499-3506.
Ardayfio, P., Moon, J., Leung, K. K., Youn-Hwang, D., Kim, K. S., (2008) Neurobiol Dis 31, 406.
Arenas, E. (2008). Foxa2: the rise and fall of dopamine neurons. Cell Stem Cell 2, 110-112.
Bjorklund, A., and Lindvall, O. (1984). Dopamine-containing systems in the CNS. In Handbook of Chemical Neuroanatomy (Amsterdam, Elsevier).
Castelo-Branco, G., Rawal, N., and Arenas, E. (2004). GSK-3beta inhibition/beta-catenin stabilization in ventral midbrain precursors increases differentiation into dopamine neurons. J Cell Sci 117, 5731-5737.
Chambers, I., Colby, D., Robertson, M., Nichols, J., Lee, S., Tweedie, S., and Smith, A. (2003). Functional expression cloning of Nanog, a pluripotency sustaining factor in mbryonic stem cells. Cell 113, 643-655.

Chi, C. L., Martinez, S., Wurst, W., and Martin, G. R. (2003). The isthmic organizer signal FGF8 is required for cell survival in the prospective midbrain and cerebellum. Development 130, 2633-2644.

Chung, S., Sonntag, K. C., Andersson, T., Bjorklund, L. M., Park, J. J., Kim, D. W., Kang, U. J., Isacson, O., and Kim, K. S. (2002). Genetic engineering of mouse embryonic stem cells by Nurr1 enhances differentiation and maturation into dopaminergic neurons. Eur J Neurosci 16, 1829-1838.
Dahl, J. A., and Collas, P. (2008). A rapid micro chromatin immunoprecipitation assay (microChIP). Nat Protoc 3, 1032-1045.
Ferri, A. L., Lin, W., Mavromatakis, Y. E., Wang, J. C., Sasaki, H., Whitsett, J. A., and Ang, S. L. (2007). Foxa1 and Foxa2 regulate multiple phases of midbrain dopaminergic neuron development in a dosage-dependent manner. Development 134, 2761-2769.
Frankel, A. D., Bredt, D. S., Pabo, C. O., 1988a Science 240, 70.
Frankel, A. D., Pabo, C. O., 1988b Cell 55, 1189.
Gassmann, M., Donoho, G., and Berg, P. (1995). Maintenance of an extrachromosomal plasmid vector in mouse embryonic stem cells. Proc Natl Acad Sci USA 92, 1292-1296.
Guo, C., Qiu, H. Y., Huang, Y., Chen, H., Yang, R. Q., Chen, S. D., Johnson, R. L., Chen, Z. F., and Ding, Y. Q. (2007). Lmx1b is essential for Fgf8 and Wnt1 expression in the isthmic organizer during tectum and cerebellum development in mice. Development 134, 317-325.
Hobert, O., and Westphal, H. (2000). Functions of LIM-homeobox genes. Trends Genet. 16, 75-83.
Hwang D. Y. et al., (2005) J Neurosci 25, 2132.
Jeong, Y., and Epstein, D. J. (2003). Distinct regulators of Shh transcription in the floor plate and notochord indicate separate origins for these tissues in the mouse node. Development 130, 3891-3902.
Joksimovic, M., Yun, B. A., Kittappa, R., Anderegg, A. M., Chang, W. W., Taketo, M. M., McKay, R. D., and Awatramani, R. B. (2009). Wnt antagonism of Shh facilitates midbrain floor plate neurogenesis. Nat Neurosci 12, 125-131.
Kawasaki, H., Mizuseki, K., Nishikawa, S., Kaneko, S., Kuwana, Y., Nakanishi, S., Nishikawa, S. I., and Sasai, Y. (2000). Induction of midbrain dopaminergic neurons from ES cells by stromal cell-derived inducing activity. Neuron 28, 31-40.
Kele, J., Simplicio, N., Ferri, A. L., Mira, H., Guillemot, F., Arenas, E., and Ang, S. L. (2006). Neurogenin 2 is required for the development of ventral midbrain dopaminergic neurons. Development 133, 495-505.
Kim, J. H., Auerbach, J. M., Rodriguez-Gomez, J. A., Velasco, I., Gavin, D., Lumelsky, N., Kim D. et al., (2009) Cell Stem Cell 4, 472.
Lee, S. H., Nguyen, J., Sanchez-Pernaute, R., Bankiewicz, K., et al. (2002). Dopamine neurons derived from embryonic stem cells function in an animal model of Parkinson's disease. Nature 418, 50-56.
Kittappa, R., Chang, W. W., Awatramani, R. B., and McKay, R. D. (2007). The foxa2 gene controls the birth and spontaneous degeneration of dopamine neurons in old age. PLoS Biol 5, e325.
Kurokawa, D., Kiyonari, H., Nakayama, R., Kimura-Yoshida, C., Matsuo, I., and Aizawa, S. (2004). Regulation of Otx2 expression and its functions in mouse forebrain and midbrain. Development 131, 3319-3331.
Lang, A. E., and Lozano, A. M. (1998). Parkinson's disease. First of two parts. N Engl J Med 339, 1044-1053.
Lee, S. M., Danielian, P. S., Fritzsch, B., and McMahon, A. P. (1997). Evidence that FGF8 signalling from the midbrain-hindbrain junction regulates growth and polarity in the developing midbrain. Development 124, 959-969.
Liu, A., and Joyner, A. L. (2001). EN and GBX2 play essential roles downstream of FGF8 in patterning the mouse mid/hindbrain region. Development 128, 181-191.
Matsunaga, E., Katahira, T., and Nakamura, H. (2002). Role of Lmx1b and Wnt1 in mesencephalon and metencephalon development. Development 129, 5269-5277.
McMahon, A. P., and Bradley, A. (1990). The Wnt-1 (int-1) proto-oncogene is required for development of a large region of the mouse brain. Cell 62, 1073-1085.
McMahon, A. P., Joyner, A. L., Bradley, A., and McMahon, J. A. (1992). The midbrain-hindbrain phenotype of Wnt-1-/Wnt-1-mice results from stepwise deletion of engrailed-expressing cells by 9.5 days postcoitum. Cell 69, 581-595.
Megason, S. G., and McMahon, A. P. (2002). A mitogen gradient of dorsal midline Wnts organizes growth in the CNS. Development 129, 2087-2098.
Millonig, J. H., Millen, K. J., and Hatten, M. E. (2000). The mouse Dreher gene Lmx1a controls formation of the roof plate in the vertebrate CNS. Nature 403, 764-769.
Ono, Y., Nakatani, T., Sakamoto, Y., Mizuhara, E., Minaki, Y., Kumai, M., Hamaguchi, A., Nishimura, M., Inoue, Y., Hayashi, H., et al. (2007). Differences in neurogenic potential in floor plate cells along an anteroposterior location: midbrain dopaminergic neurons originate from mesencephalic floor plate cells. Development 134, 3213-3225.
Ory, D. S., Neugeboren, B. A., and Mulligan, R. C. (1996). A stable human-derived packaging cell line for production of high titer retrovirus/vesicular stomatitis virus G pseudotypes. Proc Natl Acad Sci USA 93, 11400-11406.
Pabst, O., Herbrand, H., Takuma, N., and Arnold, H.-H. (2000). NKX2 gene expression in neuroectoderm but not in mesendodermally derived structures depends on sonic hedgehog in mouse embryos. Development Genes and Evolution 210, 47-50.
Prakash, N., Brodski, C., Naserke, T., Puelles, E., Gogoi, R., Hall, A., Panhuysen, M., Echevarria, D., Sussel, L., Weisenhorn, D. M., et al. (2006). A Wnt1-regulated genetic network controls the identity and fate of midbrain-dopaminergic progenitors in vivo. Development 133, 89-98.
Pruszak, J., Sonntag, K. C., Aung, M. H., Sanchez-Pernaute, R., and Isacson, O. (2007). Markers and methods for cell sorting of human embryonic stem cell-derived neural cell populations. Stem Cells 25, 2257-2268.
Saarimaki-Vire, J., Peltopuro, P., Lahti, L., Naserke, T., Blak, A. A., Vogt Weisenhorn, D. M., Yu, K., Ornitz, D. M., Wurst, W., and Partanen, J. (2007). Fibroblast growth factor receptors cooperate to regulate neural progenitor properties in the developing midbrain and hindbrain. J Neurosci 27, 8581-8592.
Sasaki, H., Hui, C., Nakafuku, M., and Kondoh, H. (1997). A binding site for Gli proteins is essential for HNF-3beta floor plate enhancer activity in transgenics and can respond to Shh in vitro. Development 124, 1313-1322.
Smidt, M. P., and Burbach, J. P. (2007). How to make a mesodiencephalic dopaminergic neuron. Nat Rev Neurosci 8, 21-32.
Smidt, M. P., Asbreuk, C. H., Cox, J. J., Chen, H., Johnson, R. L., and Burbach, J. P. (2000). A second independent pathway for development of mesencephalic dopaminergic neurons requires Lmx1b. Nat. Neurosci 3, 337-341.
Tang, M., Miyamoto, Y., and Huang, E. J. (2009). Multiple roles of beta-catenin in controlling the neurogenic niche for midbrain dopamine neurons. Development 136, 2027-2038.
Vernay, B., Koch, M., Vaccarino, F., Briscoe, J., Simeone, A., Kageyama, R., and Ang, S. L. (2005). Otx2 regulates subtype specification and neurogenesis in the midbrain. J Neurosci 25, 4856-4867.
Vokes, S. A., Ji, H., McCuine, S., Tenzen, T., Giles, S., Zhong, S., Longabaugh, W. J., Davidson, E. H., Wong, W. H., and McMahon, A. P. (2007). Genomic characterization of Gli-activator targets in sonic hedgehog-mediated neural patterning. Development 134, 1977-1989.
Ye, W., Shimamura, K., Rubenstein, J. L., Hynes, M. A., and Rosenthal, A. (1998). FGF and Shh signals control dopaminergic and serotonergic cell fate in the anterior neural plate. Cell 93, 755-766.
Yochum, G. S., McWeeney, S., Rajaraman, V., Cleland, R., Peters, S., and Goodman, R. H. (2007). Serial analysis of chromatin occupancy identifies beta-catenin target genes in colorectal carcinoma cells. Proc Natl Acad Sci USA 104, 3324-3329.

Unless otherwise defined, all technical and scientific terms used herein have the same meaning as commonly understood by one of ordinary skill in the art to which this invention belongs. All nucleotide sequences provided herein are presented in the 5′ to 3′ direction.

The inventions illustratively described herein may suitably be practiced in the absence of any element or elements, limitation or limitations, not specifically disclosed herein. Thus, for example, the terms “comprising”, “including,” containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed.

Thus, it should be understood that although the present invention has been specifically disclosed by preferred embodiments and optional features, modification, improvement and variation of the inventions embodied therein herein disclosed may be resorted to by those skilled in the art, and that such modifications, improvements and variations are considered to be within the scope of this invention. The materials, methods, and examples provided here are representative of preferred embodiments, are exemplary, and are not intended as limitations on the scope of the invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

In addition, where features or aspects of the invention are described in terms of Markush groups, those skilled in the art will recognize that the invention is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

Other embodiments are set forth within the following claims.

1. A method for treating Parkinson's Disease in a patient, said method comprising increasing the level of at least one protein selected from the group consisting of Wnt1, Lmx1a, Lmx1b, Otx2 and Pitx3 and at least one protein selected from the group consisting of SHH, FoxA2 and Nurr1 in the midbrain dopaminergic neurons of said patient, wherein the increased biological activity of said proteins is sufficient to treat Parkinson's Disease.

2. The method of claim 1, wherein said midbrain dopaminergic neurons are located in the substantia nigra A9 region.

3. The method of claim 1, wherein the level of FoxA2, Lmx1a and Otx2 is increased in the midbrain dopaminergic neurons of said patient.

4. The method of claim 1, wherein the level of Nurr1, Pitx3 and Lmx1a is increased in the midbrain dopaminergic neurons of said patient.

5. The method of claim 1, wherein the level of Nurr1, Pitx3, Lmx1a, FoxA2 and Otx2 is increased in the midbrain dopaminergic neurons of said patient.

6. The method of claim 1, wherein said method comprises administering to said patient a vector comprising a polynucleotide encoding at least one of the said proteins, operably linked to a promoter, wherein neurons in the patient take up said vector and express said protein.

7. The method of claim 6, wherein said vector is a viral vector.

8. The method of claim 7, wherein said viral vector is selected from the group consisting of an adenovirus, adeno-associated virus, lentivirus, and retrovirus.

9. The method of claim 6, wherein said vector is administered to the substantia nigra.

10. The method of claim 1, wherein said method comprises administering said proteins to said patient.

11. The method of claim 10, wherein at least one protein is encapsulated.

12. The method of claim 10, wherein at least one protein is chemically or recombinantly linked to a cell penetrating peptide (CPP).

13. The method of claim 12, wherein said cell penetrating peptide is from about 15 to about 25 amino acid residues long.

14. The method of claim 12, wherein said cell penetrating peptide comprises at least three basic amino acids selected from the group consisting of arginine, lysine, histidine and combinations thereof.

15. (canceled)

16. The method of claim 12, wherein the cell penetrating peptide comprises a HIV-TAT peptide.

17. The method of claim 1, wherein said method further comprises increasing the biological activity of at least one of the proteins selected from the group consisting of En1, En2 and Ngn2 in the midbrain dopaminergic neurons of said patient.

18. A method for producing a neural cell comprising:
(a) providing a progenitor cell, and
(b) increasing the level of at least one protein selected from the group consisting of Wnt1, Lmx1a, Lmx1b, Otx2 and Pitx3 and at least one protein selected from the group consisting of SHH, FoxA2 and Nurr1 in said progenitor cell under conditions suitable to produce a neural cell.

19. The method of claim 18, wherein said neural cell is a dopaminergic neuron.

20. The method of claim 18, wherein said neural cell expresses tyrosine hydroxylase.

21. The method of claim 18, wherein said neural cell expresses the dopamine transporter or dopa decarboxylase (DDC).

22.-53. (canceled)
